SEQUENCE LISTING (127 sequences)

SEQ ID NO: 1 - DNA, 21 bp, Artificial Sequence
  Description of Artificial Sequence Synthetic oligonucleotide
ggacaatagagatcatctagt

SEQ ID NO: 2 - DNA, 58 bp, Artificial Sequence
  Description of Artificial Sequence Synthetic oligonucleotide
ccggggacaatagagatcatctagtctcgagactagatgatctctattgtcctttttg

SEQ ID NO: 3 - DNA, 58 bp, Artificial Sequence
  Description of Artificial Sequence Synthetic oligonucleotide
aattcaaaaaggacaatagagatcatctagtctcgagactagatgatctctattgtcc

SEQ ID NO: 4 - DNA, 58 bp, Artificial Sequence
  Description of Artificial Sequence Synthetic oligonucleotide
ccggggacattccaagcctcttaacctcgaggttaagaggcttggaatgtcctttttg

SEQ ID NO: 5 - DNA, 58 bp, Artificial Sequence
  Description of Artificial Sequence Synthetic oligonucleotide
aattcaaaaaggacattccaagcctcttaacctcgaggttaagaggcttggaatgtcc

SEQ ID NO: 6 - DNA, 58 bp, Artificial Sequence
  Description of Artificial Sequence Synthetic oligonucleotide
ccggcatggtttcagagaaattatgctcgagcataatttctctgaaaccatgtttttg

SEQ ID NO: 7 - DNA, 58 bp, Artificial Sequence
  Description of Artificial Sequence Synthetic oligonucleotide
aattcaaaaacatggtttcagagaaattatgctcgagcataatttctctgaaaccatg

SEQ ID NO: 8 - DNA, 21 bp, Artificial Sequence
  Description of Artificial Sequence Synthetic oligonucleotide
cctaaggttaagtcgccctcg

SEQ ID NO: 9 - protein, 839 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 10 - protein, 612 aa, Homo sapiens [sequence omitted]

SEQ ID NO: 11 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 12 - protein, 477 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGFVLFHKIPLDG

SEQ ID NO: 13 - RNA, 22 nt, Artificial Sequence
  Description of Artificial Sequence Synthetic oligonucleotide
cuauacaaccuacugccuuccc

SEQ ID NO: 14 - RNA, 22 nt, Artificial Sequence
  Description of Artificial Sequence Synthetic oligonucleotide
ugagguaguagguugugugguu

SEQ ID NO: 15 - RNA, 22 nt, Artificial Sequence
  Description of Artificial Sequence Synthetic oligonucleotide
uggaauguaaagaaguauguau

SEQ ID NO: 16 - RNA, 22 nt, Artificial Sequence
  Description of Artificial Sequence Synthetic oligonucleotide
acauacuucuuuauaugcccau

SEQ ID NO: 17 - RNA, 22 nt, Artificial Sequence
  Description of Artificial Sequence Synthetic oligonucleotide
auaaagcuagauaaccgaaagu

SEQ ID NO: 18 - RNA, 23 nt, Artificial Sequence
  Description of Artificial Sequence Synthetic oligonucleotide
ucuuugguuaucuagcuguauga

SEQ ID NO: 19 - RNA, 22 nt, Artificial Sequence
  Description of Artificial Sequence Synthetic oligonucleotide
caaauucguaucuaggggaaua

SEQ ID NO: 20 - RNA, 23 nt, Artificial Sequence
  Description of Artificial Sequence Synthetic oligonucleotide
uacccuguagauccgaauuugug

SEQ ID NO: 21 - RNA, 22 nt, Artificial Sequence
  Description of Artificial Sequence Synthetic oligonucleotide
caggccauauugugcugccuca

SEQ ID NO: 22 - RNA, 22 nt, Artificial Sequence
  Description of Artificial Sequence Synthetic oligonucleotide
uagcagcacauaaugguuugug

SEQ ID NO: 23 - RNA, 22 nt, Artificial Sequence
  Description of Artificial Sequence Synthetic oligonucleotide
ccaguauuaacugugcugcuga

SEQ ID NO: 24 - RNA, 22 nt, Artificial Sequence
  Description of Artificial Sequence Synthetic oligonucleotide
ccaauauuacugugcugcuuua

SEQ ID NO: 25 - RNA, 22 nt, Artificial Sequence
  Description of Artificial Sequence Synthetic oligonucleotide
uagcagcacguaaauauuggcg

SEQ ID NO: 26 - RNA, 22 nt, Artificial Sequence
  Description of Artificial Sequence Synthetic oligonucleotide
acugcagugaaggcacuuguag

SEQ ID NO: 27 - RNA, 23 nt, Artificial Sequence
  Description of Artificial Sequence Synthetic oligonucleotide
caaagugcuuacagugcagguag

SEQ ID NO: 28 - RNA, 23 nt, Artificial Sequence
  Description of Artificial Sequence Synthetic oligonucleotide
acugcccuaagugcuccuucugg

SEQ ID NO: 29 - RNA, 23 nt, Artificial Sequence
  Description of Artificial Sequence Synthetic oligonucleotide
uaaggugcaucuagugcagauag

SEQ ID NO: 30 - RNA, 22 nt, Artificial Sequence
  Description of Artificial Sequence Synthetic oligonucleotide
acugcauuaugagcacuuaaag

SEQ ID NO: 31 - RNA, 23 nt, Artificial Sequence
  Description of Artificial Sequence Synthetic oligonucleotide
uaaagugcuuauagugcagguag

SEQ ID NO: 32 - RNA, 22 nt, Artificial Sequence
  Description of Artificial Sequence Synthetic oligonucleotide
uggcucaguucagcaggaacag

SEQ ID NO: 33 - RNA, 22 nt, Artificial Sequence
  Description of Artificial Sequence Synthetic oligonucleotide
ugccuacugagcugauaucagu

SEQ ID NO: 34 - RNA, 22 nt, Artificial Sequence
  Description of Artificial Sequence Synthetic oligonucleotide
ugccuacugagcugaaacacag

SEQ ID NO: 35 - RNA, 22 nt, Artificial Sequence
  Description of Artificial Sequence Synthetic oligonucleotide
cuuucagucggauguuuacagc

SEQ ID NO: 36 - RNA, 22 nt, Artificial Sequence
  Description of Artificial Sequence Synthetic oligonucleotide
uguaaacauccuugacuggaag

SEQ ID NO: 37 - RNA, 22 nt, Artificial Sequence
  Description of Artificial Sequence Synthetic oligonucleotide
acugcugagcuagcacuucccg

SEQ ID NO: 38 - RNA, 23 nt, Artificial Sequence
  Description of Artificial Sequence Synthetic oligonucleotide
caaagugcuguucgugcagguag

SEQ ID NO: 39 - RNA, 23 nt, Artificial Sequence
  Description of Artificial Sequence Synthetic oligonucleotide
agcagcauuguacagggcuauga

SEQ ID NO: 40 - RNA, 23 nt, Artificial Sequence
  Description of Artificial Sequence Synthetic oligonucleotide
agcuucuuuacagugcugccuug

SEQ ID NO: 41 - RNA, 23 nt, Artificial Sequence
  Description of Artificial Sequence Synthetic oligonucleotide
ucauagcccuguacaaugcugcu

SEQ ID NO: 42 - RNA, 22 nt, Artificial Sequence
  Description of Artificial Sequence Synthetic oligonucleotide
cugcaauguaagcacuucuuac

SEQ ID NO: 43 - RNA, 23 nt, Artificial Sequence
  Description of Artificial Sequence Synthetic oligonucleotide
aaaagugcuuacagugcagguag

SEQ ID NO: 44 - RNA, 22 nt, Artificial Sequence
  Description of Artificial Sequence Synthetic oligonucleotide
ccgcacuguggguacuugcugc

SEQ ID NO: 45 - RNA, 21 nt, Artificial Sequence
  Description of Artificial Sequence Synthetic oligonucleotide
uaaagugcugacagugcagau

SEQ ID NO: 46 - RNA, 23 nt, Artificial Sequence
  Description of Artificial Sequence Synthetic oligonucleotide
agcagcauuguacagggcuauca

SEQ ID NO: 47 - RNA, 22 nt, Artificial Sequence
  Description of Artificial Sequence Synthetic oligonucleotide
acaggugagguucuugggagcc

SEQ ID NO: 48 - RNA, 24 nt, Artificial Sequence
  Description of Artificial Sequence Synthetic oligonucleotide
ucccugagacccuuuaaccuguga

SEQ ID NO: 49 - RNA, 22 nt, Artificial Sequence
  Description of Artificial Sequence Synthetic oligonucleotide
acggguuaggcucuugggagcu

SEQ ID NO: 50 - RNA, 22 nt, Artificial Sequence
  Description of Artificial Sequence Synthetic oligonucleotide
ucacaagucaggcucuugggac

SEQ ID NO: 51 - RNA, 22 nt, Artificial Sequence
  Description of Artificial Sequence Synthetic oligonucleotide
ucccugagacccuaacuuguga

SEQ ID NO: 52 - RNA, 21 nt, Artificial Sequence
  Description of Artificial Sequence Synthetic oligonucleotide
ucacagugaaccggucucuuu

SEQ ID NO: 53 - RNA, 23 nt, Artificial Sequence
  Description of Artificial Sequence Synthetic oligonucleotide
cggggccguagcacugucugaga

SEQ ID NO: 54 - RNA, 23 nt, Artificial Sequence
  Description of Artificial Sequence Synthetic oligonucleotide
gggggccgauacacuguacgaga

SEQ ID NO: 55 - RNA, 22 nt, Artificial Sequence
  Description of Artificial Sequence Synthetic oligonucleotide
uuugguccccuucaaccagcug

SEQ ID NO: 56 - RNA, 22 nt, Artificial Sequence
  Description of Artificial Sequence Synthetic oligonucleotide
agcugguaaaauggaaccaaau

SEQ ID NO: 57 - RNA, 22 nt, Artificial Sequence
  Description of Artificial Sequence Synthetic oligonucleotide
uuugguccccuucaaccagcua

SEQ ID NO: 58 - RNA, 22 nt, Artificial Sequence
  Description of Artificial Sequence Synthetic oligonucleotide
uaacacugucugguaaagaugg

SEQ ID NO: 59 - RNA, 22 nt, Artificial Sequence
  Description of Artificial Sequence Synthetic oligonucleotide
caucuuccaguacaguguugga

SEQ ID NO: 60 - RNA, 21 nt, Artificial Sequence
  Description of Artificial Sequence Synthetic oligonucleotide
agggagggacgggggcugugc

SEQ ID NO: 61 - RNA, 23 nt, Artificial Sequence
  Description of Artificial Sequence Synthetic oligonucleotide
ucuggcuccgugucuucacuccc

SEQ ID NO: 62 - RNA, 21 nt, Artificial Sequence
  Description of Artificial Sequence Synthetic oligonucleotide
ugguucuagacuugccaacua

SEQ ID NO: 63 - RNA, 24 nt, Artificial Sequence
  Description of Artificial Sequence Synthetic oligonucleotide
uuuggcaaugguagaacucacacu

SEQ ID NO: 64 - RNA, 21 nt, Artificial Sequence
  Description of Artificial Sequence Synthetic oligonucleotide
cucccacaugcaggguuugca

SEQ ID NO: 65 - RNA, 21 nt, Artificial Sequence
  Description of Artificial Sequence Synthetic oligonucleotide
caucccuugcaugguggaggg

SEQ ID NO: 66 - RNA, 22 nt, Artificial Sequence
  Description of Artificial Sequence Synthetic oligonucleotide
gguccagaggggagauagguuc

SEQ ID NO: 67 - RNA, 22 nt, Artificial Sequence
  Description of Artificial Sequence Synthetic oligonucleotide
uaacacugucugguaacgaugu

SEQ ID NO: 68 - RNA, 22 nt, Artificial Sequence
  Description of Artificial Sequence Synthetic oligonucleotide
caucuuaccggacagugcugga

SEQ ID NO: 69 - RNA, 22 nt, Artificial Sequence
  Description of Artificial Sequence Synthetic oligonucleotide
uaauacugccugguaaugauga

SEQ ID NO: 70 - RNA, 22 nt, Artificial Sequence
  Description of Artificial Sequence Synthetic oligonucleotide
caucuuacugggcagcauugga

SEQ ID NO: 71 - RNA, 21 nt, Artificial Sequence
  Description of Artificial Sequence Synthetic oligonucleotide
gcugggaaggcaaagggacgu

SEQ ID NO: 72 - RNA, 22 nt, Artificial Sequence
  Description of Artificial Sequence Synthetic oligonucleotide
uucccuuugucauccuaugccu

SEQ ID NO: 73 - RNA, 22 nt, Artificial Sequence
  Description of Artificial Sequence Synthetic oligonucleotide
uggaauguaaggaagugugugg

SEQ ID NO: 74 - RNA, 23 nt, Artificial Sequence
  Description of Artificial Sequence Synthetic oligonucleotide
agcuacauugucugcuggguuuc

SEQ ID NO: 75 - RNA, 22 nt, Artificial Sequence
  Description of Artificial Sequence Synthetic oligonucleotide
accuggcauacaauguagauuu

SEQ ID NO: 76 - RNA, 22 nt, Artificial Sequence
  Description of Artificial Sequence Synthetic oligonucleotide
gaggguuggguggaggcucucc

SEQ ID NO: 77 - RNA, 21 nt, Artificial Sequence
  Description of Artificial Sequence Synthetic oligonucleotide
agggcccccccucaauccugu

SEQ ID NO: 78 - RNA, 23 nt, Artificial Sequence
  Description of Artificial Sequence Synthetic oligonucleotide
cgcauccccuagggcauuggugu

SEQ ID NO: 79 - RNA, 20 nt, Artificial Sequence
  Description of Artificial Sequence Synthetic oligonucleotide
ccucugggcccuuccuccag

SEQ ID NO: 80 - RNA, 23 nt, Artificial Sequence
  Description of Artificial Sequence Synthetic oligonucleotide
gcaaagcacacggccugcagaga

SEQ ID NO: 81 - RNA, 21 nt, Artificial Sequence
  Description of Artificial Sequence Synthetic oligonucleotide
gccccugggccuauccuagaa

SEQ ID NO: 82 - RNA, 22 nt, Artificial Sequence
  Description of Artificial Sequence Synthetic oligonucleotide
cuagguauggucccagggaucc

SEQ ID NO: 83 - RNA, 22 nt, Artificial Sequence
  Description of Artificial Sequence Synthetic oligonucleotide
uccgucucaguuacuuuauagc

SEQ ID NO: 84 - RNA, 22 nt, Artificial Sequence
  Description of Artificial Sequence Synthetic oligonucleotide
uuauaaagcaaugagacugauu

SEQ ID NO: 85 - RNA, 22 nt, Artificial Sequence
  Description of Artificial Sequence Synthetic oligonucleotide
gcccugaacgaggggucuggag

SEQ ID NO: 86 - RNA, 22 nt, Artificial Sequence
  Description of Artificial Sequence Synthetic oligonucleotide
gcugacuccuaguccagggcuc

SEQ ID NO: 87 - RNA, 22 nt, Artificial Sequence
  Description of Artificial Sequence Synthetic oligonucleotide
cuuaucagauuguauuguaauu

SEQ ID NO: 88 - RNA, 22 nt, Artificial Sequence
  Description of Artificial Sequence Synthetic oligonucleotide
uuauaauacaaccugauaagug

SEQ ID NO: 89 - RNA, 22 nt, Artificial Sequence
  Description of Artificial Sequence Synthetic oligonucleotide
cuuagcagguuguauuaucauu

SEQ ID NO: 90 - RNA, 22 nt, Artificial Sequence
  Description of Artificial Sequence Synthetic oligonucleotide
auauaauacaaccugcuaagug

SEQ ID NO: 91 - RNA, 22 nt, Artificial Sequence
  Description of Artificial Sequence Synthetic oligonucleotide
cacuuagcagguuguauuauau

SEQ ID NO: 92 - RNA, 22 nt, Artificial Sequence
  Description of Artificial Sequence Synthetic oligonucleotide
auaauacaaccugcuaagugcu

SEQ ID NO: 93 - RNA, 20 nt, Artificial Sequence
  Description of Artificial Sequence Synthetic oligonucleotide
auuccuagaaauuguucaua

SEQ ID NO: 94 - RNA, 23 nt, Artificial Sequence
  Description of Artificial Sequence Synthetic oligonucleotide
acuucaccugguccacuagccgu

SEQ ID NO: 95 - RNA, 23 nt, Artificial Sequence
  Description of Artificial Sequence Synthetic oligonucleotide
uggucgaccaguuggaaaguaau

SEQ ID NO: 96 - RNA, 22 nt, Artificial Sequence
  Description of Artificial Sequence Synthetic oligonucleotide
acuggacuuagggucagaaggc

SEQ ID NO: 97 - RNA, 22 nt, Artificial Sequence
  Description of Artificial Sequence Synthetic oligonucleotide
uggcaguguauuguuagcuggu

SEQ ID NO: 98 - RNA, 22 nt, Artificial Sequence
  Description of Artificial Sequence Synthetic oligonucleotide
cagccacaacuacccugccacu

SEQ ID NO: 99 - RNA, 22 nt, Artificial Sequence
  Description of Artificial Sequence Synthetic oligonucleotide
aggcaguguauuguuagcuggc

SEQ ID NO: 100 - RNA, 23 nt, Artificial Sequence
  Description of Artificial Sequence Synthetic oligonucleotide
uugcuaguugcacuccucucugu

SEQ ID NO: 101 - RNA, 25 nt, Artificial Sequence
  Description of Artificial Sequence Synthetic oligonucleotide
uaggcaguguauugcuagcggcugu

SEQ ID NO: 102 - RNA, 22 nt, Artificial Sequence
  Description of Artificial Sequence Synthetic oligonucleotide
gucauacacggcucuccucucu

SEQ ID NO: 103 - RNA, 22 nt, Artificial Sequence
  Description of Artificial Sequence Synthetic oligonucleotide
ugauugguacgucuguggguag

SEQ ID NO: 104 - RNA, 21 nt, Artificial Sequence
  Description of Artificial Sequence Synthetic oligonucleotide
uacugcagacaguggcaauca

SEQ ID NO: 105 - RNA, 22 nt, Artificial Sequence
  Description of Artificial Sequence Synthetic oligonucleotide
uacugcagacguggcaaucaug

SEQ ID NO: 106 - RNA, 22 nt, Artificial Sequence
  Description of Artificial Sequence Synthetic oligonucleotide
agacuucccauuugaagguggc

SEQ ID NO: 107 - RNA, 22 nt, Artificial Sequence
  Description of Artificial Sequence Synthetic oligonucleotide
gacuauagaacuuucccccuca

SEQ ID NO: 108 - RNA, 21 nt, Artificial Sequence
  Description of Artificial Sequence Synthetic oligonucleotide
agggggaaaguucuauagucc

SEQ ID NO: 109 - RNA, 21 nt, Artificial Sequence
  Description of Artificial Sequence Synthetic oligonucleotide
uggaggagaaggaaggugaug

SEQ ID NO: 110 - protein, 825 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 111 - protein, 831 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPVDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 112 - protein, 817 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPVDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 113 - protein, 612 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTWVFSNIDNHGILNLKDNRDHLVPSTHYIYEEPEVQSGEVSYPRSHGFREIMLNPISLPGHSKPLNHGIYVEDVNVYFSKGRHGF

SEQ ID NO: 114 - RNA, 21 nt, Artificial Sequence
  Description of Artificial Sequence Synthetic oligonucleotide
gguauaagugcacacugaaua

SEQ ID NO: 115 - RNA, 21 nt, Artificial Sequence
  Description of Artificial Sequence Synthetic oligonucleotide
cacugaauagucuaaucuaca

SEQ ID NO: 116 - RNA, 21 nt, Artificial Sequence
  Description of Artificial Sequence Synthetic oligonucleotide
gagagucaaacaauguuaagg

SEQ ID NO: 117 - RNA, 21 nt, Artificial Sequence
  Description of Artificial Sequence Synthetic oligonucleotide
gagaagaguucuaugguuauc

SEQ ID NO: 118 - RNA, 21 nt, Artificial Sequence
  Description of Artificial Sequence Synthetic oligonucleotide
gacaaagcagugugcucuaau

SEQ ID NO: 119 - RNA, 21 nt, Artificial Sequence
  Description of Artificial Sequence Synthetic oligonucleotide
ggauguguuuguacuugcaga

SEQ ID NO: 120 - protein, 838 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 121 - protein, 553 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTWPRGSPKTA

SEQ ID NO: 122 - protein, 537 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTWPRGSPKTA

SEQ ID NO: 123 - protein, 464 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHYHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGFVLFHKIPLDG

SEQ ID NO: 124 - DNA, 20 bp, Artificial Sequence
  Description of Artificial Sequence Synthetic primer
ggattctgtttgacatgggg

SEQ ID NO: 125 - DNA, 20 bp, Artificial Sequence
  Description of Artificial Sequence Synthetic primer
tccagtcaggactcaaaccc

SEQ ID NO: 126 - DNA, 20 bp, Artificial Sequence
  Description of Artificial Sequence Synthetic primer
accacagtccatgccatcac

SEQ ID NO: 127 - DNA, 20 bp, Artificial Sequence
  Description of Artificial Sequence Synthetic primer
tccaccaccctgttgctgta

(19) United States
(12) Patent Application Publication
(10) Pub. No.: US 2017/0029823 A1
(43) Pub. Date: Feb. 2, 2017

(54) TRUNCATED TRK B AND TRK C ANTAGONISTS AND USES THEREOF

(71) Applicants: Horacio Uri Saragovi, Montreal (CA); Marc Robert Fabian, Beaconsfield (CA)

(72) Inventors: Horacio Uri Saragovi, Montreal (CA); Marc Robert Fabian, Beaconsfield (CA)

(21) Appl. No.: 15/222,752

(22) Filed: Jul. 28, 2016

Related U.S. Application Data
(60) Provisional application No. 62/198,055, filed on Jul. 28, 2015.

(51) Int. Cl. C12N 15/113

(52) U.S. Cl. CPC C12N 15/1138; C12N 2320/30; C12N 2310/531; C12N 2310/14

(57) ABSTRACT

Disclosed herein are compositions, methods, vectors, and kits comprising an antagonist of a truncated TrkC or a truncated TrkB. Also described herein are methods of diagnosing, treating and/or preventing a neurodegenerative disease or condition or a symptomatic or pre-symptomatic condition with alterations to synapses associated with an elevated expression level of a truncated TrkC or truncated TrkB isoform.

### CROSS-REFERENCE

This application claims the benefit of U.S. Provisional Application No. 62/198,055, filed Jul. 28, 2015, which application is incorporated herein by reference

### SEQUENCE LISTING

The instant application contains a Sequence Listing which has been submitted electronically in ASCII format and is hereby incorporated by reference in its entirety. Said ASCII copy, created on Jul. 28, 2016, is named 6452728_Canada_Inc_701_201_SL.txt and is 98,084 bytes in size.

### BACKGROUND OF THE INVENTION

Neuron loss such as motor neuron loss is the cause of morbidity and mortality in many neurodegenerative diseases and spinal cord traumatic pathologies such as amyotrophic lateral sclerosis (ALS), spinal muscular atrophy (SMA), or spinal cord injury (SCI). In some instances, motor neurons express Trk family of receptors such as TrkB and/or TrkC. Activity of the Trk family of receptors, mediated by the kinase catalytic domain, correlates with neuron survival or death, maintenance of synapses and phenotype, and function. In some instances in a disease setting, Trk receptors are expressed as truncated Trk isoforms which are deleterious to neuronal health by inhibiting the neuroprotective action mediated by the kinase catalytic domain of Trk, and by activating neurotoxic pathways.

### SUMMARY OF THE INVENTION

Disclosed herein, in certain embodiments, is a pharmaceutical composition comprising (a) a nucleic acid polymer that comprises at least 80% sequence identity to a nucleic acid sequence selected from SEQ ID NOs: 1-5 and 114-119, and wherein the nucleic acid polymer is at most 100 nucleotides in length; and (b) a pharmaceutically acceptable excipient and/or a delivery vehicle. In some embodiments, the nucleic acid polymer comprises at least 85%, 90%, 95%, or 99% sequence identity to a nucleic acid sequence selected from SEQ ID NOs: 1-5 and 114-119, and wherein the nucleic acid polymer is at most 100 nucleotides in length. In some embodiments, the nucleic acid polymer comprises 100% sequence identity to a nucleic acid sequence selected from SEQ ID NOs: 1-5 and 114-119, and wherein the nucleic acid polymer is at most 100 nucleotides in length. In some embodiments, the nucleic acid polymer consists of a nucleic acid sequence selected from SEQ ID NOs: 1-5 and 114-119. In some embodiments, the nucleic acid polymer hybridizes to a target sequence of the truncated TrkC or truncated TrkB mRNA. In some embodiments, the nucleic acid polymer induces a decrease in a truncated TrkC or truncated TrkB expression level or a decrease in truncated TrkC or truncated TrkB activity. In some embodiments, the nucleic acid polymer induces a decrease in a truncated TrkC or truncated TrkB expression level or a decrease in truncated TrkC or truncated TrkB activity in glial cells, Müller cells, astrocytes, macrophages, or pro-inflammatory immune cells. In some embodiments, the decrease in truncated TrkC or truncated TrkB expression or activity correlates to a decrease in TNF-α production. In some embodiments, the nucleic acid polymer comprising at least 80%, 85%, 90%, 95%, or 99% sequence identity to a nucleic acid sequence selected from SEQ ID NOs: 1-5 hybridizes to a target sequence of the truncated TrkC. In some embodiments, the nucleic acid polymer comprising 100% sequence identity to a nucleic acid sequence selected from SEQ ID NOs: 1-5 hybridizes to a target sequence of the truncated TrkC. In some embodiments, the nucleic acid polymer consisting of a nucleic acid sequence selected from SEQ ID NOs: 1-5 hybridizes to a target sequence of the truncated TrkC. In some embodiments, the nucleic acid polymer comprising at least 80%, 85%, 90%, 95%, or 99% sequence identity to a nucleic acid sequence that hybridizes to a target sequence of the truncated TrkB. In some embodiments, the nucleic acid polymer comprising 100% sequence identity to a nucleic acid sequence that hybridizes to a target sequence of the truncated TrkB. In some embodiments, the nucleic acid polymer comprising at least 80%, 85%, 90%, 95%, or 99% sequence identity to a nucleic acid sequence selected from SEQ ID NOs: 114-119 hybridizes to a target sequence of the truncated TrkB. In some embodiments, the nucleic acid polymer comprising 100% sequence identity to a nucleic acid sequence selected from SEQ ID NOs: 114-119 hybridizes to a target sequence of the truncated TrkB. In some embodiments, the nucleic acid polymer consisting of a nucleic acid sequence selected from SEQ ID NOs: 114-119 hybridizes to a target sequence of the truncated TrkB. In some embodiments, the nucleic acid polymer is further modified at the nucleoside moiety, at the phosphate moiety, or a combination thereof. In some embodiments, the nucleic acid polymer further comprises one or more artificial nucleotide bases. In some embodiments, the one or more artificial nucleotide bases comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, locked nucleic acid (LNA), ethylene nucleic acid (ENA), peptide nucleic acid (PNA), l′, 5′-anhydrohexitol nucleic acids (HNA), morpholino, methylphosphonate nucleotides, thiolphosphonate nucleotides, or 2′-fluoro N3-P5′-phosphoramidites. In some embodiments, the nucleic acid polymer comprises a short hairpin RNA (shRNA) molecule, a microRNA (miRNA) molecule, or a siRNA molecule. In some embodiments, the nucleic acid polymer further comprises a complement nucleic acid polymer to form a double stranded RNA molecule. In some embodiments, the nucleic acid polymer is at most 10, 15, 20, 25, 30, 35, 40, 45, 50, 55, 60, 70, 80, 90, or 100 nucleotides in length. In some embodiments, the pharmaceutical composition comprising the nucleic acid polymer is administered to a patient in need thereof as an intramuscular, intrathecal, intravitreal, intraconjunctival, intravenous or subcutaneous administration. In some embodiments, the composition further comprises a vector that comprises the nucleic acid polymer. In some embodiments, the vector is a viral vector. In some embodiments, the viral vector is a lentiviral vector. In some embodiments, the truncated TrkC is a non-catalytic truncated TrkC. In some embodiments, the truncated TrkC protein comprises at least 80%, 85%, 90%, 95%, or 99% sequence identity to the amino acid sequence of SEQ ID NO: 10 or 113. In some embodiments, the truncated TrkC protein consists of the amino acid sequence of SEQ ID NO: 10 or 113. In some embodiments, the truncated TrkC is TrkC.T1. In some embodiments, the truncated TrkB is a non-catalytic truncated TrkB. In some embodiments, the truncated TrkB protein comprises at least 80%, 85%, 90%, 95%, or 99% sequence identity to an amino acid sequence selected from SEQ ID NOs: 11-12 and 121-123. In some embodiments, the truncated TrkB protein consists of an amino acid sequence selected from SEQ ID NOs: 11-12 and 121-123. In some embodiments, the truncated TrkB is TrkB.T1. In some embodiments, the pharmaceutical composition is administered for the treatment of a non-otic disease or condition associated with an elevated expression level or activity of truncated TrkC or truncated TrkB.

Disclosed herein, in certain embodiments, is a vector comprising a nucleic acid polymer wherein the nucleic acid polymer comprises at least 80%, 85%, 90%, 95%, or 99% sequence identity to a nucleic acid sequence selected from SEQ ID NOs: 1-5 and 114-119. In some embodiments, the vector comprises a nucleic acid polymer wherein the nucleic acid polymer consists of a nucleic acid sequence selected from SEQ ID NOs: 1-5 and 114-119. In some embodiments, the vector is a viral vector. In some embodiments, the viral vector is a lentiviral vector, a retroviral vector, an adenoviral vector, an adeno-associated viral vector, an alphaviral vector, a herpes simplex virus vector, a vaccinia viral vector, or a chimeric viral vector. In some embodiments, the vector is delivered through a viral delivery method. In some embodiments, the vector is delivered through electroporation, chemical method, microinjection, gene gun, impalefection, hydrodynamics-based delivery, continuous infusion, or sonication. In some embodiments, the chemical method is lipofection. In some embodiments, the vector is administered to a patient in need thereof as an intramuscular, intrathecal, intravitreal, intraconjunctival, intravenous or subcutaneous administration. In some embodiments the delivery of the vector is through infection, or adsorption, or transcytosis.

Disclosed herein, in certain embodiments, is a pharmaceutical composition comprising (a) a small molecule antagonist of a truncated TrkC or truncated TrkB; and (b) a pharmaceutically acceptable excipient and/or a delivery vehicle. In some embodiments, the small molecule antagonist impedes the truncated TrkC or truncated TrkB interaction with a truncated TrkC or truncated TrkB binding partner. In some embodiments, the small molecule is a peptidomimetic. In some embodiments, the small molecule is a small molecule as illustrated in FIG. 2. In some embodiments, the truncated TrkC is a non-catalytic truncated TrkC. In some embodiments, the truncated TrkC is TrkC.T1. In some embodiments, the truncated TrkB is a non-catalytic truncated TrkB. In some embodiments, the truncated TrkB is TrkB.T1. In some embodiments, the truncated TrkC binding partner comprises a neurotropic factor or a microRNA molecule. In some embodiments, the neurotropic factor is neurotrophin-3 (NT-3). In some embodiments, the microRNA molecule comprises miR-128, miR-509, or miR-768-5p. In some embodiments, the truncated TrkB binding partner comprises a neurotropic factor. In some embodiments, the neurotropic factor is brain derived neurotrophic factor (BDNF), neurotrophin-3 (NT-3), or neurotrophin-4 (NT-4).

Disclosed herein, in certain embodiments, is a pharmaceutical composition comprising (a) a polypeptide antagonist of a truncated TrkC or truncated TrkB; and (b) a pharmaceutically acceptable excipient and/or a delivery vehicle. In some embodiments, the polypeptide antagonist is an antibody or binding fragment thereof. In some embodiments, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, single-domain antibody (sdAb), or camelid antibody or binding fragment thereof. In some embodiments, the truncated TrkC is a non-catalytic truncated TrkC. In some embodiments, the truncated TrkC is TrkC.T1. In some embodiments, the truncated TrkB is a non-catalytic truncated TrkB. In some embodiments, the truncated TrkB is TrkB.T1.

Disclosed herein, in certain embodiments, is a pharmaceutical composition comprising (a) a nucleic acid polymer that hybridizes to a target sequence comprising a binding motif selected from CCAAUC, CUCCAA, or ACUGUG, wherein the binding motif is located in a sequence encoding a truncated TrkC, and wherein the nucleic acid polymer is at most 100 nucleotides in length; and (b) a pharmaceutically acceptable excipient and/or a delivery vehicle. In some embodiments, the nucleic acid polymer hybridizes to a target sequence that is located at the 3′UTR region of the truncated TrkC mRNA. In some embodiments, the nucleic acid polymer comprises a short hairpin RNA (shRNA) molecule, a microRNA (miRNA) molecule, a siRNA molecule, or a double stranded RNA molecule. In some embodiments, the nucleic acid polymer is a shRNA molecule. In some embodiments, the truncated TrkC is a non-catalytic truncated TrkC. In some embodiments, the truncated TrkC is TrkC.T1.

Disclosed herein, in certain embodiments, is a method of treating a non-otic disease or condition associated with an elevated expression level or activity of truncated TrkC or truncated TrkB, comprising administering to a patient having a non-otic disease or condition a therapeutic amount of a pharmaceutical composition described herein. Also disclosed herein is a method of preventing a non-otic disease or condition associated with an elevated expression level or activity of truncated TrkC or truncated TrkB or reducing the progression of a non-otic disease or condition associated with an elevated expression level or activity of truncated TrkC or truncated TrkB, comprising administering to a patient having a non-otic disease or condition a therapeutic amount of a pharmaceutical composition described herein. In some embodiments, the non-otic disease or condition comprises a neurodegenerative disease or a symptomatic or pre-symptomatic condition with loss of synapses. In some embodiments, the neurodegenerative disease comprises polyglutamine expansion disorder, fragile X syndrome, fragile XE mental retardation, Friedreich's ataxia, myotonic dystrophy, spinocerebellar ataxia type 8, and spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, glaucoma, ischemia stroke, Krabbe disease, Lewy body dementia, multiple sclerosis, multiple system atrophy, Parkinson's disease, Pelizaeus-Merzbacher disease, Pick's disease, primary lateral sclerosis, retinitis pigmentosa, Refsum's disease, Sandhoff disease, Schilder's disease, spinal cord injury (SCI), spinal muscular atrophy (SMA), Steele-Richardson-Olszewski disease, and Tabes dorsalis. In some embodiments, the polyglutamine repeat disease is Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17). In some embodiments, the non-otic disease or condition comprises amyotrophic lateral sclerosis (ALS), spinal muscular atrophy (SMA), glaucoma, spinal cord injury (SCI), or retinitis pigmentosa. In some embodiments, the pharmaceutical composition is administered for intramuscular, intrathecal, intravitreal, intraconjunctival, intravenous or subcutaneous administration. In some embodiments, the pharmaceutical composition modulates the splicing enhancer elements. In some instances, the modulation comprises alternative splicing of the truncated TrkC or truncated TrkB gene. In some instances, the modulation comprises exon skipping or introduction of mutations within an exon.

Disclosed herein, in certain embodiments, is a method of treating retinitis pigmentosa, comprising administering to a patient in need thereof a therapeutic amount of a pharmaceutical composition described herein. Also disclosed herein is a method of preventing retinitis pigmentosa or reducing the progression of retinitis pigmentosa, comprising administering to a patient in need thereof a therapeutic amount of a pharmaceutical composition described herein.

Disclosed herein, in certain embodiments, is a method of treating amyotrophic lateral sclerosis (ALS), comprising administering to a patient in need thereof a therapeutic amount of a pharmaceutical composition described herein. Also disclosed herein is a method of preventing amyotrophic lateral sclerosis (ALS), or reducing the progression of amyotrophic lateral sclerosis (ALS), comprising administering to a patient in need thereof a therapeutic amount of a pharmaceutical composition described herein. In some embodiments, at least 40%, 45%, 50%, 55%, or 60% reduction in the expression level or activity of a truncated TrkC or truncated TrkB relative to a control leads to a reduced ALS onset or progression. In some embodiments, the control is the expression level or activity of a truncated TrkC or truncated TrkB in the absence of the pharmaceutical composition described herein.

Disclosed herein, in certain embodiments, is a method of stratifying an individual having a non-otic disease or condition for treatment with a pharmaceutical composition described herein, comprising: (a) determining the expression level of a truncated TrkC or truncated TrkB; and (b) administering to the individual a therapeutically effective amount of the pharmaceutical composition described herein if there is an elevated expression level of the truncated TrkC or truncated TrkB. Also described herein is a method of optimizing the therapy of an individual receiving a pharmaceutical composition described herein for treatment of a non-otic disease or condition, comprising: (a) determining the expression level of a truncated TrkC or truncated TrkB; and (b) modifying, discontinuing, or continuing the treatment based on the expression level of the truncated TrkC or truncated TrkB. In some embodiments, the method further comprises testing a sample containing nucleic acid molecules encoding the truncated TrkC or truncated TrkB obtained from the individual. In some embodiments, the nucleic acid molecule is RNA. In some embodiments, the nucleic acid molecule is DNA. In some embodiments, the DNA is genomic DNA. In some embodiments, testing comprises amplifying the nucleic acid molecules encoding the truncated TrkC or truncated TrkB gene. In some embodiments, amplification is by isothermal amplification or polymerase chain reaction (PCR). In some embodiments, amplification is by PCR. In some embodiments, the sample is a cell sample or a tissue sample.

Disclosed herein, in certain embodiments, is a method of stratifying an individual having retinitis pigmentosa for treatment with a pharmaceutical composition described herein, comprising: (a) determining the expression level of a truncated TrkC or truncated TrkB; and (b) administering to the individual a therapeutically effective amount of the pharmaceutical composition described herein if there is an elevated expression level of the truncated TrkC or truncated TrkB. Also described herein is a method of optimizing the therapy of an individual receiving a pharmaceutical composition described herein for treatment of retinitis pigmentosa, comprising: (a) determining the expression level of a truncated TrkC or truncated TrkB; and (b) modifying, discontinuing, or continuing the treatment based on the expression level of the truncated TrkC or truncated TrkB. Additionally described herein is a method of stratifying an individual having ALS for treatment with a pharmaceutical composition described herein, comprising: (a) determining the expression level of a truncated TrkC or truncated TrkB; and (b) administering to the individual a therapeutically effective amount of the pharmaceutical composition described herein if there is an elevated expression level of the truncated TrkC or truncated TrkB. Further described herein is a method of optimizing the therapy of an individual receiving a pharmaceutical composition described herein for treatment of ALS, comprising: (a) determining the expression level of a truncated TrkC or truncated TrkB; and (b) modifying, discontinuing, or continuing the treatment based on the expression level of the truncated TrkC or truncated TrkB. In some embodiments, the method further comprises testing a sample containing nucleic acid molecules encoding the truncated TrkC or truncated TrkB obtained from the individual. In some embodiments, the nucleic acid molecule is RNA. In some embodiments, the nucleic acid molecule is DNA. In some embodiments, the DNA is genomic DNA. In some embodiments, testing comprises amplifying the nucleic acid molecules encoding the truncated TrkC or truncated TrkB gene. In some embodiments, amplification is by isothermal amplification or polymerase chain reaction (PCR). In some embodiments, amplification is by PCR. In some embodiments, the sample is a cell sample or a tissue sample.

Disclosed herein, in certain embodiments, is a pharmaceutical composition comprising: (a) an antagonist of truncated TrkC or truncated TrkB, wherein the antagonist induces a decrease in the truncated TrkC or truncated TrkB expression level or activity or impedes the truncated TrkC or truncated TrkB interaction with a truncated TrkC or truncated TrkB binding partner; and (b) a pharmaceutically acceptable excipient and/or a delivery vehicle.

Also disclosed herein, in certain embodiments, is a pharmaceutical composition comprising: (a) a nucleic acid polymer that hybridizes to a target sequence of truncated TrkC or truncated TrkB, wherein the nucleic acid polymer is capable of decreasing the expression level of the truncated TrkC or truncated TrkB; and (b) a pharmaceutically acceptable excipient and/or a delivery vehicle. In some embodiments, the target sequence is located at the 3′UTR region of the truncated TrkC or truncated TrkB mRNA.

Further disclosed herein, in certain embodiments, is a method of decreasing the expression of a truncated TrkC or truncated TrkB in a cell in vivo, comprising contacting the cell with a composition comprising an antagonist of truncated TrkC or truncated TrkB, wherein the antagonist induces a decrease in the truncated TrkC or truncated TrkB expression level. In some embodiments, the cell is a glial cell. In some embodiments, the glial cell is an astrocyte, a microglia, or a Müller glia.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A and FIG. 1B illustrate the full length TrkC and TrkB and their respective isoforms. FIG. 1A is adapted from Esteban et al. J Cell Biol 2006; 173:291-299. FIG. 1B is adapted from Luberg et al. J. Neurochem. 2010; 113:952-964.

FIG. 2 illustrates exemplary small molecule antagonists of truncated TrkC. Compound 3F is used in some biological assays.

FIG. 3 illustrates a graphical model of the location and the role of TrkC-FL and TrkC.T1 in motor neuron disease. Full length TrkC (TrkC-FL) is present in the neuronal cell body in the spinal cord and at the neuronal terminals in the Neuromuscular Junction (NMJ) in the periphery. Target muscle (and local glia) produces NT-3. In a ligand-dependent manner TrkC-FL sends “positive” signals that regulate neuronal cell physiology, maintain motor neuron phenotype, maintain synapses at the neuromuscular junction, and support neuronal function, and survival through mediators such as p-Akt and p-PLCγ. Activated TrkC-FL at the NMJ is transported long distances retrogradely to the neuronal cell body, in the activated state. Truncated TrkC (TrkC.T1) is expressed de novo in activated astrocytes in the spinal cord, early in disease states. In a ligand-dependent manner, TrkC.T1 sends “negative” signals that increase inflammatory cytokines that are neurotoxic. In some instances, it is possible that TrkC.T1 is expressed in Schwann cells or in activated astrocytes lining the axons outside the spinal cord. The p75^{NTR} mediates pleiotrophic effects including neuronal death in ALS and is not shown to keep the graph simple. NT-3 also activates p75^{NTR}, which causes microglia activation.

FIG. 4A and FIG. 4B illustrates that in ALS there is de novo expression of TrkC.T1 mRNA and protein in spinal cord astrocytes. FIG. 4A. TrkC.T1 mRNA expression. Combined fluorescent in situ mRNA hybridization (red, for TrkC.T1 mRNA) with immunofluorescence (green, GFAP marker). 40× magnification of 20 μm-thick sections of the lumbar spinal cord of G93A ALS mice. The probe specific for TrkC.T1 encompasses exons 13b & 14b as well as some of the 3′UTR. The anti-GFAP is a rabbit polyclonal from Millipore. The data shows nearly complete co-localization of TrkC.T1 mRNA and GFAP. FIG. 4B. TrkC.T1 protein expression. Co-localization the anti-TrkC.T1 antibody 750 and the anti-GFAP antibody in sections from G93A ALS mice (top panels) or wild type mice (bottom panels). 63× magnification of spinal cord sections 12 μm thick.

FIG. 5A-FIG. 5E illustrates the expression of TrkC.T1 in human ALS and mouse ALS, and correlation with TNF-α levels. FIG. 5A TrkC.T1 mRNA is up-regulated in spinal cords obtained from human donors with sporadic ALS (n=5), compared versus non-ALS control (n=7). FIG. 5B in the same donors a concomitant reduction in the levels of micro-RNA miR128 which is known to destabilize the spliced TrkC.T1 mRNA. Data is normalized to the reference RNU6 (small nuclear RNA). FIG. 5C There is an increase in TrkC.T1 mRNA in the spinal cord of the symptomatic G93A mice (n>9 per group). FIG. 5D in symptomatic mice there is a reduction in the levels of micro-RNA miR128. FIG. 5E these events correlate with increased TNF-α mRNA in ALS mouse spinal cord (n>9 per group).

FIG. 6A-FIG. 6D illustrates the expression and role of TrkC.T1 in glaucomatous neuronal death. FIG. 6A shows Western blot analysis of whole retinas dissected from mutant or WT mice (normal eyes or day 14 glaucomatous eyes), with an antiserum directed against the juxtamembrane domain of the trkC receptor (serum 656). An anti-actin specific antibody was used to control for loading. None of the specific bands of the expected MW are seen when the primary serum is replaced with control normal rabbit serum. FIG. 6B shows sustained increase of intraocular pressure (TOP, glaucoma). The right eyes (open symbols) were cauterized to increase TOP, the left eyes (filled symbols) are normal control. The differences in TOP between the right and left eyes was significantly different at all of the times shown (p<0.05). Sustained high TOP is the same in wt, heterozygous TrkC.T1 KO, or homozygous TrkC.T1 KO mice. FIG. 6C shows live RGCs were counted in normal retinas (OS, standardized to 100%) or glaucomatous (OD) retinas after 35 days of constant ocular hypertension. All animals in a group were averaged±sem, n=as indicated. FIG. 6D shows elevation of TNF-α and α2M neurotoxic cytokines in wild type mice with glaucoma but not in TrkC.T1^{-/-} mice with glaucoma. The right eye (OD) of wild type (n=4 mice), or TrkC.T1^{-/-} (n=4 mice) mice were cauterized to induce glaucoma for 14 days. The contralateral eyes (OS) serve as internal controls. Expression of TNF-α and α2M for each eye was adjusted to β-actin levels, and the OD/OS ratio was calculated. Data for each group are averaged±sem, n=4.

FIG. 7A-FIG. 7D illustrates development and validation of a TrkC.T1 inhibitor that prevents TrkC.T1-mediated induction of TNF-α FIG. 7A The mRNA levels of TrkC-FL and TrkC.T1 were quantified by PCR, after infection with lentivirus pLKO.Scr (scrambled control) or with pLKO.1 (expressing a unique shRNA (SEQ ID NO: 1) targeting TrkC.T1 mRNA specifically). Results are normalized to reference GAPDH mRNA. Data are expressed as the mean+SEM; n=3 independent experiments each in triplicate. The inset shows a western blot of TrkC expression for samples prepared from HEK293-TrkC-FL or HEK293-TrkC.T1 cells treated with pLKO.Scr (control) or pLKO.1, standardized to actin. FIG. 7B and FIG. 7C In rMC-1 cells the levels of TNF-α mRNA and protein were quantified by PCR or by ELISA after infection with pLKO.1TrkC.T1 or pLKOScr control. NT-3 and LPS treatment induce TNF-α and pLKO.1 prevents it. Data are standardized to untreated control. Average±sem, n=3 independent experiments each in triplicate. FIG. 7D In spinal cord of ALS G93A versus wild type mice the levels of TNF-α mRNA were assessed by quantitative PCR. Results were normalized to GAPDH. Data are expressed as the mean±SEM n=8 independent spinal cords per group. A-F t-test was applied for statistical analysis. * p<0.05, ** p<0.01 versus control. Stars with brackets indicate differences between the indicated groups.

FIG. 8A-FIG. 8C illustrates TrkC.T1 depletion delays degeneration of the ONL during RP. FIG. 8A ONL average thickness measurements (±SEM) from FD-OCT images at post-natal days 18, 20, 24 and 28. The ONL thickness decreases over time in RHOP:TrkC.T1^{+/+} retinas compared to WT. Note that the thinning of the retina is delayed in RHOP:TrkC.T1+/− retinas compared with RHOP:TrkC.T1+/+, n=8-10 mice per group, *p<0.001 (RHOP:TrkC.T1+/+ versus WT) and #p<0.01 (RHOP:TrkC.T1+/+ versus RHOP:TrkC.T1+/−). FIG. 8B FD-OCT representative retina images at post-natal days 20 and 24, scale bar=30 μm (top). Quantification of the ONL thickness measurements from FD-OCT images (±SEM) at post-natal day 18, 20, 24, and 28; n=8-10 mice per group, *p<0.01, **p<0.001 (bottom). FIG. 8C Representative images of ultrathin retinal sections at post natal days 20 and 24, scale bar=60 μm (top). Quantification of the number of photoreceptors per mm2 (average±SEM) in ultrathin retinal sections. A total of 18 images were taken from n=2 retinas per group, *p<0.01. Images were taken at 40×. NGI: Nerve fiber layer—Ganglion cell layer—Inner plexiform layer; IPL: inner plexiform layer; INL: inner nuclear layer; ONL: outer nuclear layer; IS/OS: internal/external segment.

FIG. 9A-FIG. 9B illustrates TrkC.T1 mRNA location in healthy and RHOP retinas. FIG. 9A In situ mRNA hybridization with TrkC.T1 sense probe (negative control) in WT and RHOP:TrkC.T1^{+/+} mice at post-natal day 24. FIG. 9B In situ mRNA hybridization with TrkC.T1 antisense probe in WT, RHOP:TrkC.T1^{+/+} and RHOP:TrkC.T1^{+/-} mice at post-natal day 24. Histogram shows the quantification of the area of TrkC.T1 signal in the different retina layers. TrkC.T1 was increased in the GCL and INL in RHOP:TrkC.T1^{+/+} retinas and partially decreased in RHOP:TrkC.T1^{+/-}. Data are represented as normalization of the area values of RHOP:TrkC.T1^{+/+} and RHOP:TrkC.T1^{+/-} to the WT area values (±SEM). A total of 18 images were taken from an n=3-4 retinas per group, *p<0.01. Images were taken at 20×. Scale bar=25 μm. GCL: ganglion cell layer; IPL: inner plexiform layer; INL: inner nuclear layer; ONL: outer nuclear layer; PhR: photoreceptor layer.

FIG. 10A-FIG. 10B illustrates TrkC.T1 protein location in healthy and RHOP retinas. FIG. 10A Representative images of TrkC.T1 immunoreactivity in WT, RHOP:TrkC.T1^{+/+} and RHOP:TrkC.T1^{+/-} mice at post-natal day 24. Images were taken at 20×. Scale bar=25 μm. FIG. 10B Quantification of TrkC.T1 immunoreactivity in the different retina layers at post-natal day 24. TrkC.T1 immunoreactivity was increased in the GCL, IPL, INL and PhR of RHOP:TrkC.T1^{+/+} mice retinas whereas TrkC.T1 was decreased in RHOP:TrkC.T1^{+/-} eyes. Data are shown as the average area in pixels (±SEM) (top) and as normalized area values of RHOP:TrkC.T1^{+/+} and RHOP:TrkC.T1^{+/-} to the WT area values (±SEM). A total of 30-40 images were taken from an n=3-4 retinas per group, # p<0.01 and * p<0.001 (bottom). GCL: ganglion cell layer; IPL: inner plexiform layer; INL: inner nuclear layer; ONL: outer nuclear layer; PhR: photoreceptor layer.

FIG. 11A-FIG. 11B illustrates Phospho-ERK is up-regulated in Müller cells in RP. FIG. 11A p-Erk immunoreactivity in WT, RHOP:TrkC.T1^{+/+} and RHOP:TrkC.T1^{+/-} at post-natal days 20, 24 and 28. P-ERK signal was markedly increased in Müller cells in RHOP-TrkC.T1′⁺ retinas at all post natal days. Note that p-Erk immunostaining was detected in the somata of Müller cells, basal-end feet and in the fibers projected towards the outer retinal layers. The p-Erk immunoreactivity was partially decreased in RHOP:TrkC.T1^{+/-} retinas FIG. 11B Quantification of p-Erk area in WT, RHOP:TrkC.T1^{+/+} and RHOP:TrkC.T1^{+/-} at post-natal days 20, 24 and 28. Data are shown as the average area in pixels (±SEM) (left) and as normalized area values (right) of RHOP:TrkC.T1^{+/+} and RHOP:TrkC.T1^{+/-} to the WT area values (±SEM). Area was increased in RHOP:TrkC.T1^{+/+} retinas at all post-natal days tested. Note that RHOP:TrkC.T1^{+/-} retinas showed a reduction of p-Erk area at post-natal days 20 and 24. No difference in area values were observed between RHOP:TrkC.T1^{+/+} and RHOP:TrkC.T1^{+/-} at post-natal day 28. 30 images taken from n=3 retinas per group, *p<0.01. GCL: ganglion cell layer; IPL: inner plexiform layer; INL: inner nuclear layer; ONL: outer nuclear layer; PhR: photoreceptor layer.

FIG. 12 illustrates Phospho-ERK co-localizes with the specific Müller cell marker glutamine synthase (GS). Representative images illustrating phospho-ERK immunoreactivity in WT, RHOP:TrkC.T1^{+/+} and RHOP:TrkC.T1^{+/-} at post-natal days 20 and 24. Phospho-ERK immunostaining was increased in RHOP:TrkC.T1^{+/+} retinas compared to the wild-type littermates at post-natal days 20 and 24. The p-Erk signal was detected in the somata of Müller cells, basal-end feet and in the fibers projected towards the photoreceptors, as shown by co-localization with GS. Note that the p-Erk immunoreactivity was partially decreased in RHOP:TrkC.T1^{+/-} retinas. Scale bar=25 μm. GCL: ganglion cell layer; IPL: inner plexiform layer; INL: inner nuclear layer; ONL: outer nuclear layer; PhR: photoreceptor layer.

FIG. 13A-FIG. 13B illustrates Phospho-Akt is up-regulated in bipolar cells in RHOP mice retinas. FIG. 13A p-Akt immunoreactivity in WT, RHOP:TrkC.T1^{+/+} and RHOP:TrkC.T1^{+/-} at post-natal days 20 and 24. P-Akt immunostaining was increased in the somata and fibers of bipolar cells in RHOP:TrkC.T1^{+/+} retinas, at post-natal day 20 and 24. P-Akt signal was partially diminished in RHOP:TrkC.T1^{+/-} retinas at post-natal day 24. FIG. 13B Quantification of p-Akt area in WT, RHOP:TrkC.T1^{+/+} and RHOP:TrkC.T1^{+/-} at post-natal days 20 and 24. Data are shown as the average area in pixels (±SEM) (left) and as normalized area values (right) of RHOP:TrkC.T1^{+/+} and RHOP:TrkC.T1^{+/-} to the WT area values (±SEM). Area was increased in RHOP:TrkC.T1^{+/+} retinas at post-natal day 24. RHOP:TrkC.T1^{+/-} retinas showed a reduction of p-Erk area at post-natal day 24. 36 images taken from n=3 retinas per group, *p<0.01. Scale bar=25 μm. GCL: ganglion cell layer; IPL: inner plexiform layer; INL: inner nuclear layer; ONL: outer nuclear layer; PhR: photoreceptor layer.

FIG. 14A-FIG. 14C illustrates TrkC antagonist compound 3F (see FIG. 2 for structure) that inhibits ONL degeneration and blocks the expression of p-Erk and p-Akt in RP. Compound 3F (1 mM, 2 μl) (right eye, treated) or vehicle (5% DMSO, 2 μl) (left eye, control) were intravitreally injected at post-natal day 17 FIG. 14A FD-OCT representative images (post-natal day 24) from WT and RHOP mice animals injected with vehicle or compound 3F. In 3F-injected RHOP eyes, ONL thickness was larger than in vehicle-injected RHOP eyes (red bar) from postnatal day 22 to post-natal day 28. No differences were observed between the 3F-injected eyes and vehicle-injected eyes in the WT mice. Histogram shows the quantification of FD-OCT images from WT and RHOP retinas at post-natal days 18, 22, 24, and 28. In each animal regardless of the genotype, the vehicle-injected eye (control) was standardized to 100%, n=3 animals per group, *p<0.01, scale bar=60 μm. FIG. 14B Western blots of RHOP mice retinas 24 hours after intravitreal injections. Histogram shows the densitometric quantification of the expression of p-Erk and p-Akt. The densitometric signal for each eye was adjusted to total ERK or total Akt, and the ratio of right/left eye was calculated. For each animal, the expression of p-Erk or p-Akt in the 3F-treated eye was standardized to the vehicle-injected eye as 100% (±SEM), *p<0.01, n=4. The comparison shows that the TrkC antagonist reduced p-Erk signal by 40% and p-Akt by 50%. FIG. 14C Representative images of p-Erk immunoreactivity in RHOP retinas at post-natal day 18. Compound 3F abolished p-Erk immunostaining compared with the vehicle-injected retinas. Histogram shows quantification of p-Erk area in pixels (±SEM), *p<0.001. Scale bar=25 μm. NGI: Nerve fiber layer-Ganglion cell layer-Inner plexiform layer; IPL: inner plexiform layer; INL: inner nuclear layer; ONL: outer nuclear layer; IS/OS: internal/external segment.

FIG. 15A-FIG. 15D illustrates TrkC.T1 directly activates MAPK/Erk in Müller cells. Representative western blots showing the levels of TrkC-FL (140 KDa), TrkC.T1 (110 kDa), p-Erk 1/2 (44 and 42 KDa) and p-Akt (60 KDa) in HEK293 cell expression system and rMC-1 cells, with or without stimulation of 4 nM NT-3 for 10 min. All membranes were stripped and reprobed with anti-actin (42 KDa) as a loading control FIG. 15A Non-infected 293-TrkC.FL cells, and 293-TrkC.T1 cells infected with the lentivirus PLKO.1^{TrkC.T1} (expressing a unique shRNA sequence that destabilizes TrkC.T1 mRNA), or the control lentivirus PLKO.1^{scrambled}, or non-infected cells. FIG. 15C Non-infected r-MC1 cells and r-MC1 cells infected with PLKO.1^{TrkC.T1} or lentivirus PLKO.1^{scrambled}. FIG. 15B, FIG. 15D Densitometric quantification of p-Erk and p-Akt signal standardized to untreated controls (arbitrary value of 1). The data represent relative protein levels±SEM. n=3 independent experiments. * p<0.05, ** p<0.01 and ***p<0.001.

FIG. 16A-FIG. 16B illustrates that MAPK/ERK inhibitor PD98059 delays ONL degeneration in RP. PD98059 (100 μM, 2 μl) (right eye) or vehicle (50% DMSO, 2 μl) (left eye, control) were intravitreally injected at post-natal day 17. FIG. 16B Representative images of p-Erk immunoreactivity in RHOP retinas at post-natal day 18. P-ERK signal was reduced in the cell bodies and to some degree in the fibers of Müller cells projected towards the PhR layer (asterisk) in RHOP eyes treated with PD98059, scale bar=25 μm. Histogram represents the quantification of the p-Erk area in pixels (±SEM), *p<0.001. FIG. 16B FD-OCT representative images (post-natal day 24) from WT and RHOP mice animals injected with vehicle or PD98059. In PD98059-injected RHOP eyes, ONL thickness was bigger than in vehicle-injected RHOP eyes (red bar) at postnatal days 24 and 28. No changes were observed in the PD98059-injected eyes compared with the vehicle-injected eyes, in WT mice. Histogram shows the quantification of FD-OCT images from WT and RHOP retinas injected with vehicle or PD9059 at post-natal days 22, 24, and 28. In each animal regardless of the genotype, the vehicle-injected eye (control) was standardized to 100%, n=3 animals per group, *P<0.05 and **p<0.01, scale bar=60 μm. NGI: Nerve fiber layer-Ganglion cell layer-Inner plexiform layer; IPL: inner plexiform layer; INL: inner nuclear layer; ONL: outer nuclear layer; IS/OS: internal/external segment.

FIG. 17A-FIG. 17B illustrates TrkC antagonist compound 3F (see FIG. 2 for structures) reduces photoreceptor death and inhibits p-Erk and p-Akt in vitro. Compound 3F (10 μM, 2 μl) (right eye, treated) or vehicle (5% DMSO, 2 μl) (left eye, control) were intravitreally injected at post-natal day 16. Retinas were dissected at post-natal day 17 and cultured for 24 h. Explants were then processed for TUNEL or Western blots. FIG. 17A Representative images of TUNEL staining of central and peripheral areas of RHOP mice retinas. Histogram shows TUNEL staining quantification of both retina areas. Data represents the number of TUNEL positive cells per mm2 (±SEM), *p<0.01, n=6. Note that 3F-treated retinas showed an increased number of photoreceptors compared to the vehicle-treated retinas. FIG. 17B Expression of p-Erk and p-Akt protein in RHOP retinas. Histogram shows densitometric analysis of Western blots. The densitometric signal for each eye was adjusted to total ERK or total Akt, and the ratio of right/left eye was calculated. For each animal, the expression of p-Erk or p-Akt in the 3F-treated eye was standardized to the vehicle-injected eye as 100% (±SEM), *p<0.01, n=3. The comparison shows that the TrkC antagonist reduced p-Erk signal by 70% and p-Akt by 40%.

### DETAILED DESCRIPTION OF THE INVENTION

Neurotrophins are dimeric polypeptide growth factors that regulate the peripheral and central nervous systems and other tissues and promote functions such as neuronal survival and regulation of synaptic plasticity. In some instances, the family of neurotrophins includes nerve growth factor (NGF), brain-derived neurotrophic factor (BDNF), neurotrophin-3 (NT-3), and neurotrophin-4 (NT-4). In some instances, neurotrophins mediate their effects through interaction with either the Trk family of receptors or with the p75 neurotrophin receptor which belongs to the tumor necrosis factor receptor superfamily. In some cases, interaction with the Trk family of receptors activates several signaling cascades, such as the phosphatidylinositol-3-kinase, phospholipase C, SN T, and Ras/mitogen-activated protein kinase pathways, which mediate growth and survival responses of the neurotrophins.

The Trk family of receptors comprises three homologs, TrkA (NTRK1), TrkB (NTRK2), and TrkC (NTRK3). Tropomyosin receptor kinase C (TrkC), also known as NT-3 growth factor receptor, neurotrophic tyrosine kinase receptor type 3, or TrkC tyrosine kinase, is the receptor for neurotrophin-3 (NT-3). Tropomyosin receptor kinase B (TrkB), also known as Tyrosine receptor kinase B, BDNF/NT-3 growth factors receptor, or neurotrophic tyrosine kinase receptor type 2, is the receptor for BDNF, NT-4, and in some instances, to NT-3 but at a reduced affinity.

In some embodiments, TrkC and TrkB comprise both full-length and truncated isoforms. In some instances, the truncated isoforms of TrkC and TrkB serve as dominant-negative regulators of their full-length counterparts. In some embodiments, the full-length TrkC and full-length TrkB are associated with neuroprotection. However in some cases, truncated TrkC and TrkB isoforms are associated with neurodegeneration or with alterations to synapses.

In some embodiments, elevated expression levels or activity of truncated TrkC or truncated TrkB are associated with a neurodegenerative disease or condition or a symptomatic or pre-symptomatic condition with alterations of synapses. For example, glaucoma is characterized by a progressive retinal ganglion cell death. In some instances, glaucoma is correlated with elevated expression of a truncated TrkC, which is a dominant-negative receptor of the full length TrkC. Indeed, it has been reported that a truncated form of TrkC isoform was upregulated during the early phase of glaucoma. The upregulation of the truncated TrkC isoform led to increased production of tumor necrosis factor-α (TNF-α), a known neurotroxic factor, whereas an inactivation of the truncated TrkC isoform led to reduced TNF-α production and promoted protection of retinal ganglion cells (Bai et al., “In glaucoma the upregulated truncated TrkC.T1 receptor isoform in glia causes increased TNF-α production, leading to retinal ganglion cell death,” Investigative ophthalmology & Visual Science, 51(12): 6639-6651 (2010)).

Described herein, in certain embodiments, are compositions, methods, vectors, and kits comprising an antagonist of a truncated TrkC or a truncated TrkB. In some embodiments, described herein is a pharmaceutical composition comprising an antagonist of truncated TrkC or truncated TrkB, wherein the antagonist induces a decrease in the truncated TrkC or truncated TrkB expression level or activity or impedes the truncated TrkC or truncated TrkB interaction with a truncated TrkC or truncated TrkB binding partner. In some embodiments, also described herein include a pharmaceutical composition comprising a nucleic acid polymer that hybridizes to a target sequence of truncated TrkC or truncated TrkB, wherein the nucleic acid polymer is capable of decreasing the expression level or the activity of the truncated TrkC or truncated TrkB.

Truncated TrkC and Truncated TrkB Isoforms

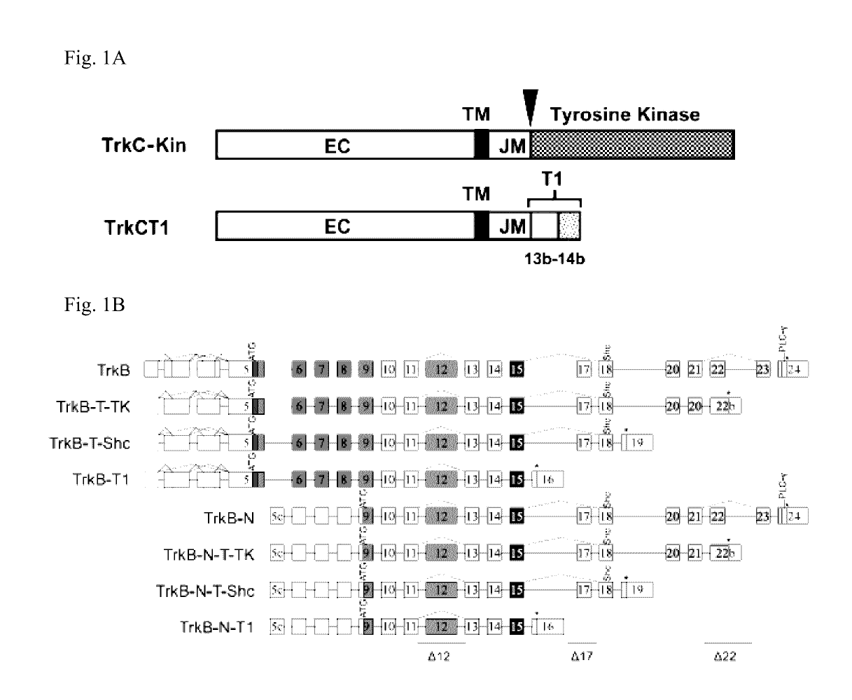

In some embodiments, TrkC comprises about 20 exons. In some instances, a truncated TrkC isoform lacks one or more of the 20 exons or comprises one or more altered exons. In some embodiments, the truncated TrkC is a non-catalytic truncated TrkC. In some embodiments, the truncated TrkC protein comprises at least 80%, 85%, 90%, 95%, or 99% sequence identity to the amino acid sequence of SEQ ID NO: 10 or 113. In some instances, the truncated TrkC protein consists of the amino acid sequence of SEQ ID NO: 10 or 113. In some cases, the truncated TrkC is TrkC.T1. In some instances, the truncated TrkC is a truncated TrkC as illustrated in FIG. 1A. In some instances, a full-length TrkC is a TrkC comprising at least 80%, 85%, 90%, 95%, 99% or 100% sequence identity to the amino acid sequence of SEQ ID NOs: 9 and 110-112.

In some instances, TrkB comprises about 24 exons. In some instances, a truncated TrkB isoform lacks one or more of the 24 exons or comprises one or more altered exons. In some instances, truncated TrkB comprises TrkB-T-TK, TrkB-T Shc, TrkB.T1 (or TrkB-T1), TrkB.T2 (or TrkB-T2), TrkB-N, TrkB-N-T-TK, TrkB-N-T-Shc, or TrkB-N-T1. In some embodiments, truncated TrkB is a truncated TrkB as illustrated in FIG. 1B.

In some embodiments, the truncated TrkB is a non-catalytic truncated TrkB. In some instances, the truncated TrkB protein comprises at least 80%, 85%, 90%, 95%, or 99% sequence identity to an amino acid sequence selected from SEQ ID NOs: 11-12 and 121-123. In some cases, the truncated TrkB protein consists of an amino acid sequence selected from SEQ ID NOs: 11-12 and 121-123. In some cases, the truncated TrkB is TrkB.T1 (or TrkB-T1). In some cases, a full-length TrkB is a TrkB comprising at least 80%, 85%, 90%, 95%, 99% or 100% sequence identity to the amino acid sequence of SEQ ID NO: 120.

### Nucleic Acid Polymer Antagonists

Disclosed herein, in certain embodiments, is a pharmaceutical composition which comprises a nucleic acid polymer. In some embodiments, disclosed herein is a pharmaceutical composition which comprises a nucleic acid polymer that comprises at least 80% sequence identity to a nucleic acid sequence selected from SEQ ID NOs: 1-5 and 114-119, and wherein the nucleic acid polymer is at most 100 nucleotides in length; and a pharmaceutically acceptable excipient and/or a delivery vehicle. In some instances, the nucleic acid polymer comprises at least 81%, 82%, 83%, 84%, 85%, 86%, 87%, 88%, 89%, 90%, 91%, 92%, 93%, 94%, 95%, 96%, 97%, 98%, or 99% sequence identity to a nucleic acid sequence selected from SEQ ID NOs: 1-5 and 114-119. In some instances, the nucleic acid polymer comprises 100% sequence identity to a nucleic acid sequence selected from SEQ ID NOs: 1-5 and 114-119. In some instances, the nucleic acid polymer consists of a nucleic acid sequence selected from SEQ ID NOs: 1-5 and 114-119.

In some cases, the nucleic acid polymer hybridizes to a target sequence of the truncated TrkC or truncated TrkB mRNA. In some cases, the nucleic acid polymer induces a decrease in a truncated TrkC or truncated TrkB expression level. In some cases, the nucleic acid polymer comprising at least 81%, 82%, 83%, 84%, 85%, 86%, 87%, 88%, 89%, 90%, 91%, 92%, 93%, 94%, 95%, 96%, 97%, 98%, or 99% sequence identity to a nucleic acid sequence selected from SEQ ID NOs: 1-5 hybridizes to a target sequence of the truncated TrkC. In some cases, the nucleic acid polymer comprising at least 81% sequence identity to a nucleic acid sequence selected from SEQ ID NOs: 1-5 hybridizes to a target sequence of the truncated TrkC. In some cases, the nucleic acid polymer comprising at least 82% sequence identity to a nucleic acid sequence selected from SEQ ID NOs: 1-5 hybridizes to a target sequence of the truncated TrkC. In some cases, the nucleic acid polymer comprising at least 83% sequence identity to a nucleic acid sequence selected from SEQ ID NOs: 1-5 hybridizes to a target sequence of the truncated TrkC. In some cases, the nucleic acid polymer comprising at least 84% sequence identity to a nucleic acid sequence selected from SEQ ID NOs: 1-5 hybridizes to a target sequence of the truncated TrkC. In some cases, the nucleic acid polymer comprising at least 85% sequence identity to a nucleic acid sequence selected from SEQ ID NOs: 1-5 hybridizes to a target sequence of the truncated TrkC. In some cases, the nucleic acid polymer comprising at least 86% sequence identity to a nucleic acid sequence selected from SEQ ID NOs: 1-5 hybridizes to a target sequence of the truncated TrkC. In some cases, the nucleic acid polymer comprising at least 87% sequence identity to a nucleic acid sequence selected from SEQ ID NOs: 1-5 hybridizes to a target sequence of the truncated TrkC. In some cases, the nucleic acid polymer comprising at least 88% sequence identity to a nucleic acid sequence selected from SEQ ID NOs: 1-5 hybridizes to a target sequence of the truncated TrkC. In some cases, the nucleic acid polymer comprising at least 89% sequence identity to a nucleic acid sequence selected from SEQ ID NOs: 1-5 hybridizes to a target sequence of the truncated TrkC. In some cases, the nucleic acid polymer comprising at least 90% sequence identity to a nucleic acid sequence selected from SEQ ID NOs: 1-5 hybridizes to a target sequence of the truncated TrkC. In some cases, the nucleic acid polymer comprising at least 91% sequence identity to a nucleic acid sequence selected from SEQ ID NOs: 1-5 hybridizes to a target sequence of the truncated TrkC. In some cases, the nucleic acid polymer comprising at least 92% sequence identity to a nucleic acid sequence selected from SEQ ID NOs: 1-5 hybridizes to a target sequence of the truncated TrkC. In some cases, the nucleic acid polymer comprising at least 93% sequence identity to a nucleic acid sequence selected from SEQ ID NOs: 1-5 hybridizes to a target sequence of the truncated TrkC. In some cases, the nucleic acid polymer comprising at least 94% sequence identity to a nucleic acid sequence selected from SEQ ID NOs: 1-5 hybridizes to a target sequence of the truncated TrkC. In some cases, the nucleic acid polymer comprising at least 95% sequence identity to a nucleic acid sequence selected from SEQ ID NOs: 1-5 hybridizes to a target sequence of the truncated TrkC. In some cases, the nucleic acid polymer comprising at least 96% sequence identity to a nucleic acid sequence selected from SEQ ID NOs: 1-5 hybridizes to a target sequence of the truncated TrkC. In some cases, the nucleic acid polymer comprising at least 97% sequence identity to a nucleic acid sequence selected from SEQ ID NOs: 1-5 hybridizes to a target sequence of the truncated TrkC. In some cases, the nucleic acid polymer comprising at least 98% sequence identity to a nucleic acid sequence selected from SEQ ID NOs: 1-5 hybridizes to a target sequence of the truncated TrkC. In some cases, the nucleic acid polymer comprising at least 99% sequence identity to a nucleic acid sequence selected from SEQ ID NOs: 1-5 hybridizes to a target sequence of the truncated TrkC. In some cases, the nucleic acid polymer comprising 100% sequence identity to a nucleic acid sequence selected from SEQ ID NOs: 1-5 hybridizes to a target sequence of the truncated TrkC. In some cases, the nucleic acid polymer consisting of a nucleic acid sequence selected from SEQ ID NOs: 1-5 hybridizes to a target sequence of the truncated TrkC.

In some instances, the nucleic acid polymer comprising at least 81%, 82%, 83%, 84%, 85%, 86%, 87%, 88%, 89%, 90%, 91%, 92%, 93%, 94%, 95%, 96%, 97%, 98%, or 99% sequence identity to a nucleic acid sequence that hybridizes to a target sequence of the truncated TrkB. In some instances, the nucleic acid polymer comprising 100% sequence identity to a nucleic acid sequence that hybridizes to a target sequence of the truncated TrkB. In some cases, the nucleic acid polymer consisting of a nucleic acid sequence that hybridizes to a target sequence of the truncated TrkB.

In some cases, the nucleic acid polymer comprising at least 81%, 82%, 83%, 84%, 85%, 86%, 87%, 88%, 89%, 90%, 91%, 92%, 93%, 94%, 95%, 96%, 97%, 98%, or 99% sequence identity to a nucleic acid sequence selected from SEQ ID NOs: 114-119 hybridizes to a target sequence of the truncated TrkB. In some cases, the nucleic acid polymer comprising at least 81% sequence identity to a nucleic acid sequence selected from SEQ ID NOs: 114-119 hybridizes to a target sequence of the truncated TrkB. In some cases, the nucleic acid polymer comprising at least 82% sequence identity to a nucleic acid sequence selected from SEQ ID NOs: 114-119 hybridizes to a target sequence of the truncated TrkB. In some cases, the nucleic acid polymer comprising at least 83% sequence identity to a nucleic acid sequence selected from SEQ ID NOs: 114-119 hybridizes to a target sequence of the truncated TrkB. In some cases, the nucleic acid polymer comprising at least 84% sequence identity to a nucleic acid sequence selected from SEQ ID NOs: 114-119 hybridizes to a target sequence of the truncated TrkB. In some cases, the nucleic acid polymer comprising at least 85% sequence identity to a nucleic acid sequence selected from SEQ ID NOs: 114-119 hybridizes to a target sequence of the truncated TrkB. In some cases, the nucleic acid polymer comprising at least 86% sequence identity to a nucleic acid sequence selected from SEQ ID NOs: 114-119 hybridizes to a target sequence of the truncated TrkB. In some cases, the nucleic acid polymer comprising at least 87% sequence identity to a nucleic acid sequence selected from SEQ ID NOs: 114-119 hybridizes to a target sequence of the truncated TrkB. In some cases, the nucleic acid polymer comprising at least 88% sequence identity to a nucleic acid sequence selected from SEQ ID NOs: 114-119 hybridizes to a target sequence of the truncated TrkB. In some cases, the nucleic acid polymer comprising at least 89% sequence identity to a nucleic acid sequence selected from SEQ ID NOs: 114-119 hybridizes to a target sequence of the truncated TrkB. In some cases, the nucleic acid polymer comprising at least 90% sequence identity to a nucleic acid sequence selected from SEQ ID NOs: 114-119 hybridizes to a target sequence of the truncated TrkB. In some cases, the nucleic acid polymer comprising at least 91% sequence identity to a nucleic acid sequence selected from SEQ ID NOs: 114-119 hybridizes to a target sequence of the truncated TrkB. In some cases, the nucleic acid polymer comprising at least 92% sequence identity to a nucleic acid sequence selected from SEQ ID NOs: 114-119 hybridizes to a target sequence of the truncated TrkB. In some cases, the nucleic acid polymer comprising at least 93% sequence identity to a nucleic acid sequence selected from SEQ ID NOs: 114-119 hybridizes to a target sequence of the truncated TrkB. In some cases, the nucleic acid polymer comprising at least 94% sequence identity to a nucleic acid sequence selected from SEQ ID NOs: 114-119 hybridizes to a target sequence of the truncated TrkB. In some cases, the nucleic acid polymer comprising at least 95% sequence identity to a nucleic acid sequence selected from SEQ ID NOs: 114-119 hybridizes to a target sequence of the truncated TrkB. In some cases, the nucleic acid polymer comprising at least 96% sequence identity to a nucleic acid sequence selected from SEQ ID NOs: 114-119 hybridizes to a target sequence of the truncated TrkB. In some cases, the nucleic acid polymer comprising at least 97% sequence identity to a nucleic acid sequence selected from SEQ ID NOs: 114-119 hybridizes to a target sequence of the truncated TrkB. In some cases, the nucleic acid polymer comprising at least 98% sequence identity to a nucleic acid sequence selected from SEQ ID NOs: 114-119 hybridizes to a target sequence of the truncated TrkB. In some cases, the nucleic acid polymer comprising at least 99% sequence identity to a nucleic acid sequence selected from SEQ ID NOs: 114-119 hybridizes to a target sequence of the truncated TrkB. In some cases, the nucleic acid polymer comprising 100% sequence identity to a nucleic acid sequence selected from SEQ ID NOs: 114-119 hybridizes to a target sequence of the truncated TrkB. In some cases, the nucleic acid polymer consisting of a nucleic acid sequence selected from SEQ ID NOs: 114-119 hybridizes to a target sequence of the truncated TrkB.

In some instances, described herein also includes a pharmaceutical composition that comprises a nucleic acid polymer that hybridizes to a target sequence comprising a binding motif selected from CCAAUC, CUCCAA, or ACUGUG, wherein the binding motif is located in a sequence encoding a truncated TrkC. In some instances, the nucleic acid polymer hybridizes to a target sequence that is located at the 3′UTR region of the truncated TrkC mRNA. In some cases, the nucleic acid polymer comprises a short hairpin RNA (shRNA) molecule, a microRNA (miRNA) molecule, a siRNA molecule, or a double stranded RNA molecule. In some cases, the nucleic acid polymer is a shRNA molecule.

In some embodiments, further described herein includes a pharmaceutical composition that comprises a nucleic acid polymer that hybridizes to a target sequence on truncated TrkC or truncated TrkB that is recognized by a microRNA (miRNA). In some instances, the miRNA comprises let-7b-3p (SEQ ID NO: 13), let-7b-5p (SEQ ID NO: 14), miR-1-3p (SEQ ID NO: 15), miR-1-5p (SEQ ID NO: 16), miR-9-3p (SEQ ID NO: 17), miR-9-5p (SEQ ID NO: 18), miR-10a-3p (SEQ ID NO: 19), miR-10a-5p (SEQ ID NO: 20), miR-15a-3p (SEQ ID NO: 21), miR-15a-5p (SEQ ID NO: 22), miR-16-1-3p (SEQ ID NO: 23), miR-16-2-3p (SEQ ID NO: 24), miR-16-5p (SEQ ID NO: 25), miR-17-3p (SEQ ID NO: 26), miR-17-5p (SEQ ID NO: 27), miR-18a-3p (SEQ ID NO: 28), miR-18a-5p (SEQ ID NO: 29), miR-20a-3p (SEQ ID NO: 30), miR-20a-5p (SEQ ID NO: 31), miR-24-3p (SEQ ID NO: 32), miR-24-1-5p (SEQ ID NO: 33), miR-24-2-5p (SEQ ID NO: 34), miR-30e-3p (SEQ ID NO: 35), miR-30e-5p (SEQ ID NO: 36), miR-93-3p (SEQ ID NO: 37), miR-93-5p (SEQ ID NO: 38), miR-103a-3p (SEQ ID NO: 39), miR-103a-2-5p (SEQ ID NO: 40), miR-103b (SEQ ID NO: 41), miR-106a-3p (SEQ ID NO: 42), miR-106a-5p (SEQ ID NO: 43), miR-106b-3p (SEQ ID NO: 44), miR-106b-5p (SEQ ID NO: 45), miR-107 (SEQ ID NO: 46), miR-125a-3p (SEQ ID NO: 47), miR-125a-5p (SEQ ID NO: 48), miR-125b-1-3p (SEQ ID NO: 49), miR-125b-2-3p (SEQ ID NO: 50), miR-125b-5p (SEQ ID NO: 51), miR-128-3p (SEQ ID NO: 52), miR-128-1-5p (SEQ ID NO: 53), miR-128-2-5p (SEQ ID NO: 54), miR-133a-3p (SEQ ID NO: 55), miR-133a-5p (SEQ ID NO: 56), miR-133b (SEQ ID NO: 57), miR-141-3p (SEQ ID NO: 58), miR-141-5p (SEQ ID NO: 59), miR-149-3p (SEQ ID NO: 60), miR-149-5p (SEQ ID NO: 61), miR-182-3p (SEQ ID NO: 62), miR-182-5p (SEQ ID NO: 63), miR-188-3p (SEQ ID NO: 64), miR-188-5p (SEQ ID NO: 65), miR-198 (SEQ ID NO: 66), miR-200a-3p (SEQ ID NO: 67), miR-200a-5p (SEQ ID NO: 68), miR-200b-3p (SEQ ID NO: 69), miR-200b-5p (SEQ ID NO: 70), miR-204-3p (SEQ ID NO: 71), miR-204-5p (SEQ ID NO: 72), miR-206 (SEQ ID NO: 73), miR-221-3p (SEQ ID NO: 74), miR-221-5p (SEQ ID NO: 75), miR-296-3p (SEQ ID NO: 76), miR-296-5p (SEQ ID NO: 77), miR-324-5p (SEQ ID NO: 78), miR-326 (SEQ ID NO: 79), miR-330-3p (SEQ ID NO: 80), miR-331-3p (SEQ ID NO: 81), miR-331-5p (SEQ ID NO: 82), miR-340-3p (SEQ ID NO: 83), miR-340-5p (SEQ ID NO: 84), miR-345-3p (SEQ ID NO: 85), miR-345-5p (SEQ ID NO: 86), miR-374a-3p (SEQ ID NO: 87), miR-374a-5p (SEQ ID NO: 88), miR-374b-3p (SEQ ID NO: 89), miR-374b-5p (SEQ ID NO: 90), miR-374c-3p (SEQ ID NO: 91), miR-374c-5p (SEQ ID NO: 92), miR-384 (SEQ ID NO: 93), miR-412-3p (SEQ ID NO: 94), miR-412-5p (SEQ ID NO: 95), miR-422a (SEQ ID NO: 96), miR-449a (SEQ ID NO: 97), miR-449b-3p (SEQ ID NO: 98), miR-449b-5p (SEQ ID NO: 99), miR-449c-3p (SEQ ID NO: 100), miR-449c-5p (SEQ ID NO: 101), miR-485-3p (SEQ ID NO: 102), miR-509-3p (SEQ ID NO: 103), miR-509-5p (SEQ ID NO: 104), miR-509-3-5p (SEQ ID NO: 105), miR-617 (SEQ ID NO: 106), miR-625-3p (SEQ ID NO: 107), miR-625-5p (SEQ ID NO: 108), miR-765 (SEQ ID NO: 109), miR-768-5p, Hsa-miR-185* or Hsa-miR-491-3p. In some cases, the nucleic acid polymer hybridizes to a target sequence on truncated TrkC or truncated TrkB that is recognized by let-7b-3p (SEQ ID NO: 13), let-7b-5p (SEQ ID NO: 14), miR-1-3p (SEQ ID NO: 15), miR-1-5p (SEQ ID NO: 16), miR-9-3p (SEQ ID NO: 17), miR-9-5p (SEQ ID NO: 18), miR-10a-3p (SEQ ID NO: 19), miR-10a-5p (SEQ ID NO: 20), miR-15a-3p (SEQ ID NO: 21), miR-15a-5p (SEQ ID NO: 22), miR-16-1-3p (SEQ ID NO: 23), miR-16-2-3p (SEQ ID NO: 24), miR-16-5p (SEQ ID NO: 25), miR-17-3p (SEQ ID NO: 26), miR-17-5p (SEQ ID NO: 27), miR-18a-3p (SEQ ID NO: 28), miR-18a-5p (SEQ ID NO: 29), miR-20a-3p (SEQ ID NO: 30), miR-20a-5p (SEQ ID NO: 31), miR-24-3p (SEQ ID NO: 32), miR-24-1-5p (SEQ ID NO: 33), miR-24-2-5p (SEQ ID NO: 34), miR-30e-3p (SEQ ID NO: 35), miR-30e-5p (SEQ ID NO: 36), miR-93-3p (SEQ ID NO: 37), miR-93-5p (SEQ ID NO: 38), miR-103a-3p (SEQ ID NO: 39), miR-103a-2-5p (SEQ ID NO: 40), miR-103b (SEQ ID NO: 41), miR-106a-3p (SEQ ID NO: 42), miR-106a-5p (SEQ ID NO: 43), miR-106b-3p (SEQ ID NO: 44), miR-106b-5p (SEQ ID NO: 45), miR-107 (SEQ ID NO: 46), miR-125a-3p (SEQ ID NO: 47), miR-125a-5p (SEQ ID NO: 48), miR-125b-1-3p (SEQ ID NO: 49), miR-125b-2-3p (SEQ ID NO: 50), miR-125b-5p (SEQ ID NO: 51), miR-128-3p (SEQ ID NO: 52), miR-128-1-5p (SEQ ID NO: 53), miR-128-2-5p (SEQ ID NO: 54), miR-133a-3p (SEQ ID NO: 55), miR-133a-5p (SEQ ID NO: 56), miR-133b (SEQ ID NO: 57), miR-141-3p (SEQ ID NO: 58), miR-141-5p (SEQ ID NO: 59), miR-149-3p (SEQ ID NO: 60), miR-149-5p (SEQ ID NO: 61), miR-182-3p (SEQ ID NO: 62), miR-182-5p (SEQ ID NO: 63), miR-188-3p (SEQ ID NO: 64), miR-188-5p (SEQ ID NO: 65), miR-198 (SEQ ID NO: 66), miR-200a-3p (SEQ ID NO: 67), miR-200a-5p (SEQ ID NO: 68), miR-200b-3p (SEQ ID NO: 69), miR-200b-5p (SEQ ID NO: 70), miR-204-3p (SEQ ID NO: 71), miR-204-5p (SEQ ID NO: 72), miR-206 (SEQ ID NO: 73), miR-221-3p (SEQ ID NO: 74), miR-221-5p (SEQ ID NO: 75), miR-296-3p (SEQ ID NO: 76), miR-296-5p (SEQ ID NO: 77), miR-324-5p (SEQ ID NO: 78), miR-326 (SEQ ID NO: 79), miR-330-3p (SEQ ID NO: 80), miR-331-3p (SEQ ID NO: 81), miR-331-5p (SEQ ID NO: 82), miR-340-3p (SEQ ID NO: 83), miR-340-5p (SEQ ID NO: 84), miR-345-3p (SEQ ID NO: 85), miR-345-5p (SEQ ID NO: 86), miR-374a-3p (SEQ ID NO: 87), miR-374a-5p (SEQ ID NO: 88), miR-374b-3p (SEQ ID NO: 89), miR-374b-5p (SEQ ID NO: 90), miR-374c-3p (SEQ ID NO: 91), miR-374c-5p (SEQ ID NO: 92), miR-384 (SEQ ID NO: 93), miR-412-3p (SEQ ID NO: 94), miR-412-5p (SEQ ID NO: 95), miR-422a (SEQ ID NO: 96), miR-449a (SEQ ID NO: 97), miR-449b-3p (SEQ ID NO: 98), miR-449b-5p (SEQ ID NO: 99), miR-449c-3p (SEQ ID NO: 100), miR-449c-5p (SEQ ID NO: 101), miR-485-3p (SEQ ID NO: 102), miR-509-3p (SEQ ID NO: 103), miR-509-5p (SEQ ID NO: 104), miR-509-3-5p (SEQ ID NO: 105), miR-617 (SEQ ID NO: 106), miR-625-3p (SEQ ID NO: 107), miR-625-5p (SEQ ID NO: 108), miR-765 (SEQ ID NO: 109), miR-768-5p, Hsa-miR-185* or Hsa-miR-491-3p. In some cases, the nucleic acid polymer hybridizes to a target sequence on truncated TrkC or truncated TrkB that is recognized by miR-128-3p (SEQ ID NO: 52), miR-128-1-5p (SEQ ID NO: 53), miR-128-2-5p (SEQ ID NO: 54) miR-509-3p (SEQ ID NO: 103), miR-509-5p (SEQ ID NO: 104), miR-509-3-5p (SEQ ID NO: 105), miR-768-5p, Hsa-miR-185* or Hsa-miR-491-3p. In some cases, the nucleic acid polymer hybridizes to a target sequence on truncated TrkC or truncated TrkB that is recognized by miR-128-3p (SEQ ID NO: 52), miR-128-1-5p (SEQ ID NO: 53), miR-128-2-5p (SEQ ID NO: 54). In some cases, the nucleic acid polymer hybridizes to a target sequence on truncated TrkC or truncated TrkB that is recognized by miR-509-3p (SEQ ID NO: 103), miR-509-5p (SEQ ID NO: 104), miR-509-3-5p (SEQ ID NO: 105). In some cases, the nucleic acid polymer hybridizes to a target sequence on truncated TrkC or truncated TrkB that is recognized by miR-768-5p. In some cases, the nucleic acid polymer hybridizes to a target sequence on truncated TrkC or truncated TrkB that is recognized by Hsa-miR-185*. In some cases, the nucleic acid polymer hybridizes to a target sequence on truncated TrkC or truncated TrkB that is recognized by Hsa-miR-491-3p. In some instances, Hsa-miR-185* and Hsa-miR-491-3p are as described in Maussion, et al., “Regulation of a truncated form of tropomyosin-related kinase B (TrkB) by Hsa-miR-185* in frontal cortex of suicide completers,” PLoS One 7(6): e39301 (2012).

In some embodiments, the nucleic acid polymer is at most 100 nucleotides in length. In some embodiments, the nucleic acid polymer is at most 5, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, or 100 nucleotides in length. In some embodiments, the nucleic acid polymer is about 10 nucleotides in length. In some embodiments, the nucleic acid polymer is about 11 nucleotides in length. In some embodiments, the nucleic acid polymer is about 12 nucleotides in length. In some embodiments, the nucleic acid polymer is about 13 nucleotides in length. In some embodiments, the nucleic acid polymer is about 14 nucleotides in length. In some embodiments, the nucleic acid polymer is about 15 nucleotides in length. In some embodiments, the nucleic acid polymer is about 16 nucleotides in length. In some embodiments, the nucleic acid polymer is about 17 nucleotides in length. In some embodiments, the nucleic acid polymer is about 18 nucleotides in length. In some embodiments, the nucleic acid polymer is about 19 nucleotides in length. In some embodiments, the nucleic acid polymer is about 20 nucleotides in length. In some embodiments, the nucleic acid polymer is about 21 nucleotides in length. In some embodiments, the nucleic acid polymer is about 22 nucleotides in length. In some embodiments, the nucleic acid polymer is about 23 nucleotides in length. In some embodiments, the nucleic acid polymer is about 24 nucleotides in length. In some embodiments, the nucleic acid polymer is about 25 nucleotides in length. In some embodiments, the nucleic acid polymer is about 26 nucleotides in length. In some embodiments, the nucleic acid polymer is about 27 nucleotides in length. In some embodiments, the nucleic acid polymer is about 28 nucleotides in length. In some embodiments, the nucleic acid polymer is about 29 nucleotides in length. In some embodiments, the nucleic acid polymer is about 30 nucleotides in length. In some embodiments, the nucleic acid polymer is about 31 nucleotides in length. In some embodiments, the nucleic acid polymer is about 32 nucleotides in length. In some embodiments, the nucleic acid polymer is about 33 nucleotides in length. In some embodiments, the nucleic acid polymer is about 34 nucleotides in length. In some embodiments, the nucleic acid polymer is about 35 nucleotides in length. In some embodiments, the nucleic acid polymer is about 36 nucleotides in length. In some embodiments, the nucleic acid polymer is about 37 nucleotides in length. In some embodiments, the nucleic acid polymer is about 38 nucleotides in length. In some embodiments, the nucleic acid polymer is about 39 nucleotides in length. In some embodiments, the nucleic acid polymer is about 40 nucleotides in length. In some embodiments, the nucleic acid polymer is about 45 nucleotides in length. In some embodiments, the nucleic acid polymer is about 50 nucleotides in length. In some embodiments, the nucleic acid polymer is about 55 nucleotides in length. In some embodiments, the nucleic acid polymer is about 60 nucleotides in length. In some embodiments, the nucleic acid polymer is about 70 nucleotides in length. In some embodiments, the nucleic acid polymer is about 80 nucleotides in length.

In some embodiments, the nucleic acid polymer is between about 10 and about 80 nucleotides in length. In some embodiments, the nucleic acid polymer is between about 10 and about 70 nucleotides in length. In some embodiments, the nucleic acid polymer is between about 10 and about 60 nucleotides in length. In some embodiments, the nucleic acid polymer is between about 10 and about 55 nucleotides in length. In some embodiments, the nucleic acid polymer is between about 10 and about 50 nucleotides in length. In some embodiments, the nucleic acid polymer is between about 10 and about 45 nucleotides in length. In some embodiments, the nucleic acid polymer is between about 10 and about 40 nucleotides in length. In some embodiments, the nucleic acid polymer is between about 10 and about 35 nucleotides in length. In some embodiments, the nucleic acid polymer is between about 10 and about 30 nucleotides in length. In some embodiments, the nucleic acid polymer is between about 10 and about 25 nucleotides in length. In some embodiments, the nucleic acid polymer is between about 10 and about 20 nucleotides in length.

In some embodiments, the nucleic acid polymer comprises a short hairpin RNA (shRNA) molecule, a microRNA (miRNA) molecule, or a siRNA molecule. In some embodiments, the nucleic acid polymer further comprises a complement nucleic acid polymer to form a double stranded RNA molecule.

In some embodiments, the nucleic acid polymer is a shRNA molecule. In some embodiments, the shRNA molecule comprises at least 80% sequence identity to a nucleic acid sequence selected from SEQ ID NOs: 1-5 and 114-119. In some instances, the shRNA molecule comprises at least 85%, 90%, 95%, or 99% sequence identity to a nucleic acid sequence selected from SEQ ID NOs: 1-5 and 114-119. In some instances, the shRNA molecule comprises at least 85% sequence identity to a nucleic acid sequence selected from SEQ ID NOs: 1-5 and 114-119. In some instances, the shRNA molecule comprises at least 90% sequence identity to a nucleic acid sequence selected from SEQ ID NOs: 1-5 and 114-119. In some instances, the shRNA molecule comprises at least 91% sequence identity to a nucleic acid sequence selected from SEQ ID NOs: 1-5 and 114-119. In some instances, the shRNA molecule comprises at least 92% sequence identity to a nucleic acid sequence selected from SEQ ID NOs: 1-5 and 114-119. In some instances, the shRNA molecule comprises at least 93% sequence identity to a nucleic acid sequence selected from SEQ ID NOs: 1-5 and 114-119. In some instances, the shRNA molecule comprises at least 94% sequence identity to a nucleic acid sequence selected from SEQ ID NOs: 1-5 and 114-119. In some instances, the shRNA molecule comprises at least 95% sequence identity to a nucleic acid sequence selected from SEQ ID NOs: 1-5 and 114-119. In some instances, the shRNA molecule comprises at least 96% sequence identity to a nucleic acid sequence selected from SEQ ID NOs: 1-5 and 114-119. In some instances, the shRNA molecule comprises at least 97% sequence identity to a nucleic acid sequence selected from SEQ ID NOs: 1-5 and 114-119. In some instances, the shRNA molecule comprises at least 98% sequence identity to a nucleic acid sequence selected from SEQ ID NOs: 1-5 and 114-119. In some instances, the shRNA molecule comprises at least 99% sequence identity to a nucleic acid sequence selected from SEQ ID NOs: 1-5 and 114-119. In some instances, the shRNA molecule comprises 100% sequence identity to a nucleic acid sequence selected from SEQ ID NOs: 1-5 and 114-119. In some instances, the shRNA molecule consists of a nucleic acid sequence selected from SEQ ID NOs: 1-5 and 114-119.

In some embodiments, the shRNA molecule hybridizes to a target sequence comprising a binding motif selected from CCAAUC, CUCCAA, or ACUGUG, wherein the binding motif is located in a sequence encoding a truncated TrkC. In some instances, the target sequence is located at the 3′UTR region of the truncated TrkC mRNA.

In some instances, the shRNA molecule is at most 100 nucleotides in length. In some embodiments, the shRNA molecule is at most 5, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, or 100 nucleotides in length. In some embodiments, the shRNA molecule is between about 10 and about 80 nucleotides in length. In some embodiments, the shRNA molecule is between about 10 and about 70 nucleotides in length. In some embodiments, the shRNA molecule is between about 10 and about 60 nucleotides in length. In some embodiments, the shRNA molecule is between about 10 and about 55 nucleotides in length. In some embodiments, the shRNA molecule is between about 10 and about 50 nucleotides in length. In some embodiments, the shRNA molecule is between about 10 and about 45 nucleotides in length. In some embodiments, the shRNA molecule is between about 10 and about 40 nucleotides in length. In some embodiments, the shRNA molecule is between about 10 and about 35 nucleotides in length. In some embodiments, the shRNA molecule is between about 10 and about 30 nucleotides in length. In some embodiments, the shRNA molecule is between about 10 and about 25 nucleotides in length. In some embodiments, the shRNA molecule is between about 10 and about 20 nucleotides in length.

In some embodiments, the nucleic acid polymer described herein modulates the splicing enhancer elements. In some instances, the modulation comprises alternative splicing of the truncated TrkC or truncated TrkB gene. In some instances, the modulation comprises exon skipping. In other instances, the modulation comprises introduction of one or more mutations within an exon. In some instances, the nucleic acid polymer upon modulation of the splicing enhancer elements prevents the expression of truncated TrkC or truncated TrkB. In some instances, the nucleic acid polymer upon modulation of the splicing enhancer elements increases processed mRNA of the full-length TrkC or TrkB.

In some embodiments, the nucleic acid polymer described herein comprises RNA, DNA or a combination thereof. In some instances, the nucleic acid polymer is a RNA polymer. In some instances, the nucleic acid polymer is further modified at the nucleoside moiety, at the phosphate moiety, or a combination thereof. In some cases, the nucleic acid polymer further comprises one or more artificial nucleotide bases.

In some instances, the one or more artificial nucleotide bases comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, locked nucleic acid (LNA), ethylene nucleic acid (ENA), peptide nucleic acid (PNA), l′, 5′-anhydrohexitol nucleic acids (HNA), morpholino, methylphosphonate nucleotides, thiolphosphonate nucleotides, or 2′-fluoro N3-P5′-phosphoramidites.

In some embodiments, the nucleic acid polymer comprises a modification at the nucleoside moiety. In some instances, the modification is at the 2′ hydroxyl group of the ribose moiety. In some instances, the modification is a 2′-O-methyl modification or a 2′-O-methoxyethyl (2′-O-MOE) modification. In some instances, the 2′-O-methyl modification adds a methyl group to the 2′ hydroxyl group of the ribose moiety whereas the 2′O-methoxyethyl modification add a methoxyethyl group to the 2′ hydroxyl group of the ribose moiety. Exemplary chemical structures of a 2′-O-methyl modification of an adenosine molecule and 2′O-methoxyethyl modification of a uridine are illustrated below.

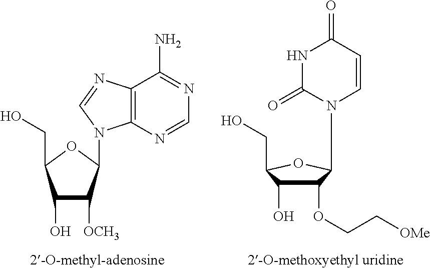

In some embodiments, an additional modification at the 2′ hydroxyl group includes a 2′-O-aminopropyl sugar conformation which involves an extended amine group comprising a propyl linker that binds the amine group to the 2′ oxygen. In some instances, this modification neutralizes the phosphate derived overall negative charge of the oligonucleotide molecule by introducing one positive charge from the amine group per sugar and thereby improve cellular uptake properties due to its zwitterionic properties. An exemplary chemical structure of a 2′-O-aminopropyl nucleoside phosphoramidite is illustrated below.

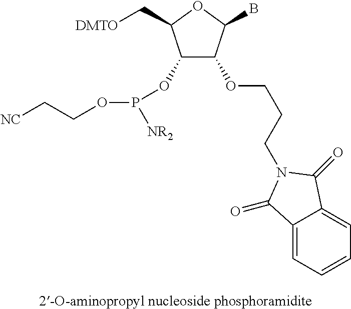

In some embodiments, the modification at the 2′ hydroxyl group includes a locked or bridged ribose conformation (e.g., locked nucleic acid or LNA) where the 4′ ribose position is also involved. In some embodiments, the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (³E) conformation of the furanose ring of an LNA monomer.

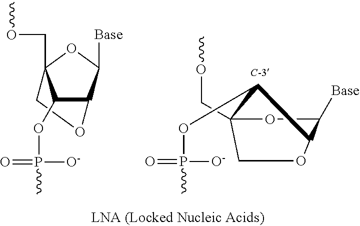

In some embodiments, a further modification at the 2′ hydroxyl group comprises ethylene nucleic acids (ENA) such as for example 2′-4′-ethylene-bridged nucleic acid, which locks the sugar conformation into a C₃′-endo sugar puckering conformation. In some instances, ENAs are part of the bridged nucleic acids class of modified nucleic acids that also comprises LNA. Exemplary chemical structures of the ENA and bridged nucleic acids are illustrated below.

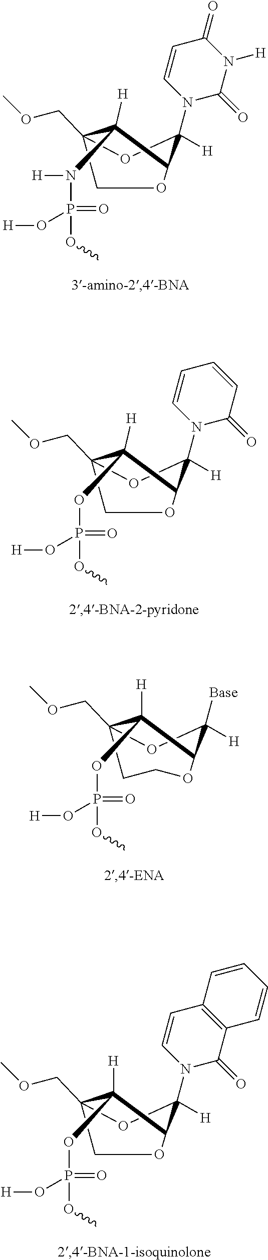

In some embodiments, still other modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, nucleotide analogues further comprise Morpholinos, peptide nucleic acids (PNAs), methylphosphonate nucleotides, thiolphosphonate nucleotides, 2′-fluoro N3-P5′-phosphoramidites, l′, 5′-anhydrohexitol nucleic acids (HNAs), or a combination thereof. In some instances, morpholino or phosphorodiamidate morpholino oligo (PMO) comprises synthetic molecules whose structure mimics natural nucleic acid structure by deviates from the normal sugar and phosphate structures. Instead, the five member ribose ring in some instances is substituted with a six member morpholino ring containing four carbons, one nitrogen and one oxygen. In some cases, the ribose monomers are linked by a phosphordiamidate group instead of a phosphate group. In some cases, these backbone alterations remove all positive and negative charges making morpholinos neutral molecules that cross cellular membranes without the aid of cellular delivery agents such as those used by charged oligonucleotides.

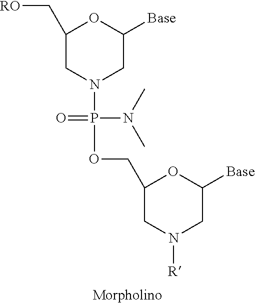

In some embodiments, peptide nucleic acid (PNA) does not contain sugar ring or phosphate linkage. Instead, the bases in some instances are attached and appropriately spaced by oligoglycine-like molecules, therefore, eliminating a backbone charge.

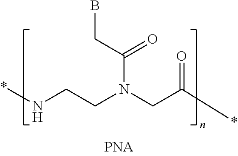

In some embodiments, modification of the phosphate backbone also comprise methyl or thiol modifications such as thiolphosphonate and methylphosphonate nucleotide. Exemplary thiolphosphonate nucleotide (left) and methylphosphonate nucleotide (right) are illustrated below.

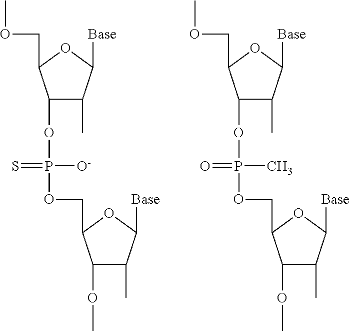

Furthermore, exemplary 2′-fluoro N3-P5′-phosphoramidites is illustrated as:

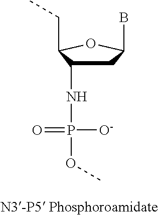

And exemplary hexitol nucleic acid (or 1′,5′-anhydrohexitol nucleic acids (HNA)) is illustrated as:

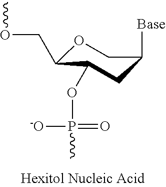

In some embodiments, also disclosed herein include a vector which comprises a nucleic acid polymer wherein the nucleic acid polymer comprises at least 80%, 85%, 90%, 95%, or 99% sequence identity to a nucleic acid sequence selected from SEQ ID NOs: 1-5 and 114-119. In some instances, the vector comprises a nucleic acid polymer wherein the nucleic acid polymer consists of a nucleic acid sequence selected from SEQ ID NOs: 1-5 and 114-119.

In some instances, the vector is a viral vector. In some instances, the viral vector is a lentiviral vector. In some cases, the vector is delivered through a viral delivery method.

In some cases, the vector is delivered through electroporation, chemical method, microinjection, gene gun, impalefection, hydrodynamics-based delivery, continuous infusion, or sonication. In some cases, the chemical method is lipofection. In some cases, the method is infection, or adsorption or transcytosis.

### Small Molecule Antagonists

Disclosed herein, in certain embodiments, also includes a pharmaceutical composition comprising a small molecule antagonists of a truncated TrkC or truncated TrkB; and a pharmaceutically acceptable excipient and/or a delivery vehicle. In some instances, the small molecule antagonist impedes the full-length TrkC/TrkB and/or truncated TrkC/TrkB interaction with a TrkC/TrkB binding partner. In some cases, the small molecule antagonist impedes the full-length TrkC or full-length TrkB interaction with a full-length TrkC or full-length TrkB binding partner. In some embodiments, the small molecule antagonist impedes the truncated TrkC or truncated TrkB interaction with a truncated TrkC or truncated TrkB binding partner.

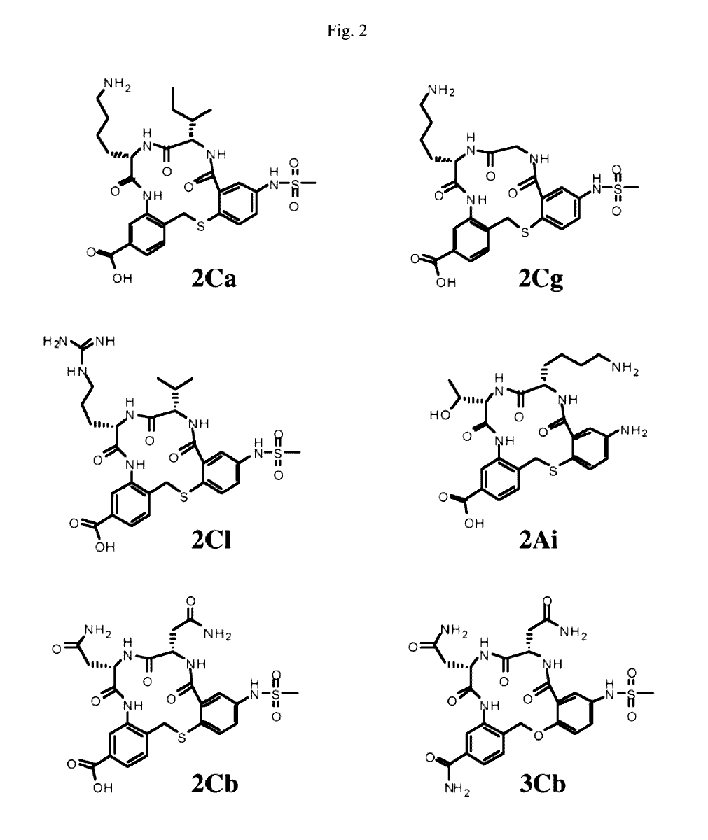

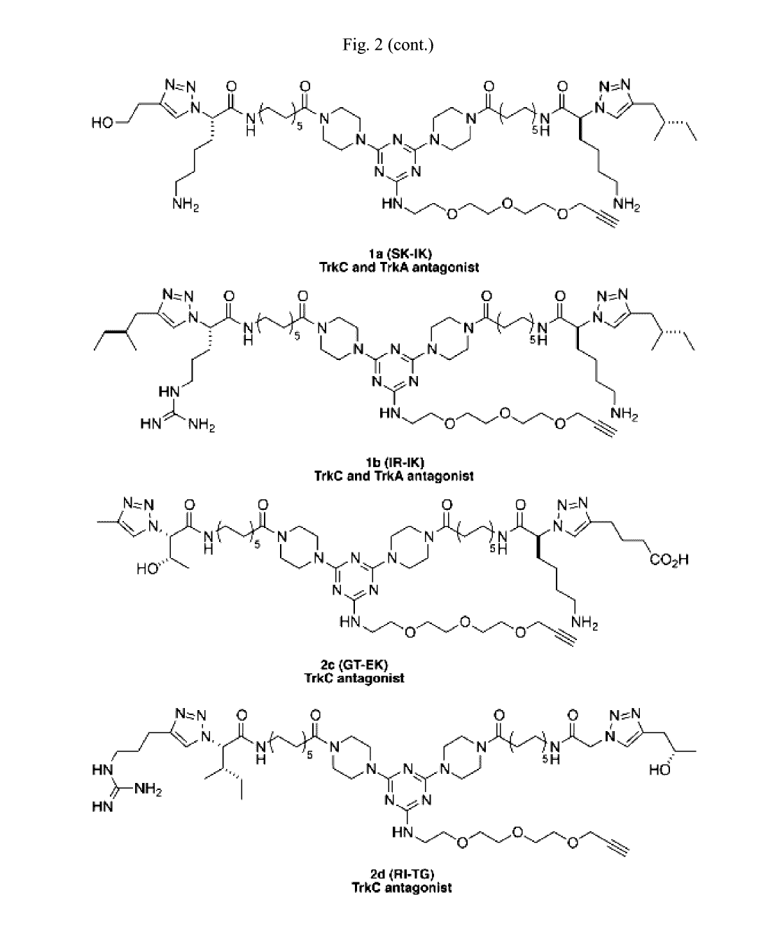

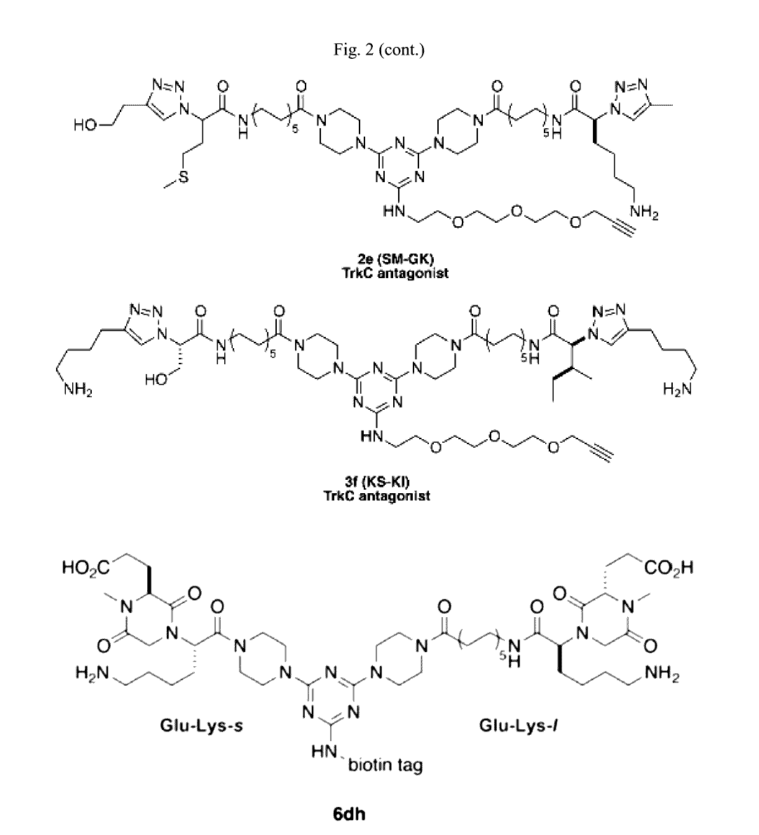

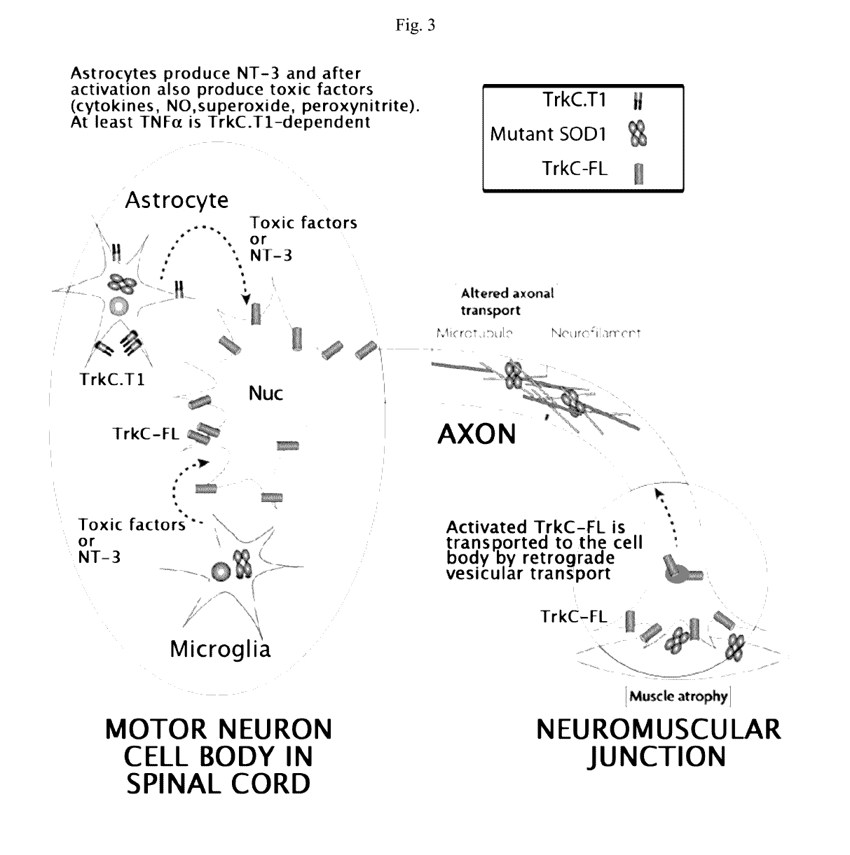

In some instances, the small molecule is a peptidomimetic. In some cases, the small molecule is a small molecule as illustrated in FIG. 2. In some embodiments, the small molecule is a small molecule antagonist described in: Brahimi et al., “A peptidomimetic of NT-3 acts as a TrkC antagonist,” Peptides 30(10):1833-1839 (2009); Liu et al., “Bivalent diketopiperazine-based tropomysin receptor kinase C (TrkC) antagonists,” J. Med. Chem. 53(13): 5044-5048 (2010); Bai et al., “In glaucoma the upregulated truncated TrkC.T1 receptor isoform in Glia causes increased TNF-α production, leading to retinal ganglion cell death,” Inv. Ophthalm. & Visual Sci. 51(12): 6639-6651 (2010); or Brahimi et al., “Combinatorial assembly of small molecules into bivalent antagonists of TrkC or TrkA receptors,” PLOS One 9(3): e89617 (2014).

In some embodiments, the small molecule antagonist is a truncated TrkC antagonist. In some embodiments, the truncated TrkC antagonist is a small molecule as illustrated in FIG. 2. In some embodiments, the truncated TrkC antagonists is a small molecule as described in: Brahimi et al., “A peptidomimetic of NT-3 acts as a TrkC antagonist,” Peptides 30(10):1833-1839 (2009); Liu et al., “Bivalent diketopiperazine-based tropomysin receptor kinase C (TrkC) antagonists,” J. Med. Chem. 53(13): 5044-5048 (2010); Bai et al., “In glaucoma the upregulated truncated TrkC.T1 receptor isoform in Glia causes increased TNF-α production, leading to retinal ganglion cell death,” Inv. Ophthalm. & Visual Sci. 51(12): 6639-6651 (2010); or Brahimi et al., “Combinatorial assembly of small molecules into bivalent antagonists of TrkC or TrkA receptors,” PLOS One 9(3): e89617 (2014).

In some instances, the truncated TrkC is a non-catalytic truncated TrkC. As described elsewhere herein, the non-catalytic truncated TrkC protein comprises at least 80%, 85%, 90%, 95%, or 99% sequence identity to the amino acid sequence of SEQ ID NO: 10 or 113. In some instances, the truncated TrkC protein consists of the amino acid sequence of SEQ ID NO: 10 or 113. In some instances, the truncated TrkC is TrkC.T1.

In some embodiments, the truncated TrkB is a non-catalytic truncated TrkB. As described elsewhere herein, the non-catalytic truncated TrkB protein comprises at least 80%, 85%, 90%, 95%, or 99% sequence identity to an amino acid sequence selected from SEQ ID NOs: 11-12 and 121-123. In some instances, the truncated TrkB protein consists of an amino acid sequence selected from SEQ ID NOs: 11-12 and 121-123. In some instances, the truncated TrkB is TrkB.T1.

In some embodiments, the truncated TrkC binding partner comprises a neurotropic factor or a microRNA molecule. In some embodiments, the neurotropic factor is neurotrophin-3 (NT-3). In some embodiments, the microRNA molecule comprises miR-128, miR-509, or miR-768-5p. In some embodiments, the truncated TrkB binding partner comprises a neurotropic factor. In some embodiments, the neurotropic factor is brain derived neurotrophic factor (BDNF), neurotrophin-3 (NT-3), or neurotrophin-4 (NT-4).

### Polypeptide Antagonists

Disclosed herein, in certain embodiments, further includes a pharmaceutical composition comprising a polypeptide antagonist of a truncated TrkC or truncated TrkB; and a pharmaceutical acceptable excipient and/or a delivery vehicle.

In some embodiments, the polypeptide antagonist is an antibody or binding fragment thereof. In some instances, the antibody or binding fragment thereof comprises a humanized antibody or binding fragment thereof, chimeric antibody or binding fragment thereof, monoclonal antibody or binding fragment thereof, a linear antibody, a single-chain antibody, a bi-specific antibody, a multispecific antibody formed from antibody fragments, a tandem antibody, a veneered antibody, monovalent Fab′, divalent Fab2, single-chain variable fragment (scFv), diabody, minibody, single-domain antibody (sdAb), a rIgG fragment, or camelid antibody or binding fragment thereof.

In some embodiments, the antibody or binding fragment thereof recognizes one or more of the epitopes of the truncated TrkC or truncated TrkB. In some embodiments, the epitopes on the truncated TrkC comprise a region within the ectodomain of the truncated TrkC. In some instances, the ectodomain of the truncated TrkC comprises the leucine-rich repeat regions and the Ig-like domain that is involved in ligand interaction. In some instances, the epitope region within the ectodomain of the truncated TrkC comprises one or more cysteine residues. In some instances, the epitope region within the ectodomain of the truncated TrkC comprises one or more cysteine residues that is capable of forming disulfide bond. In some embodiments, the antibody or binding fragment thereof recognizes one or more of the epitopes that comprises one or more of the cysteine residues.

In some embodiments, the epitopes on the truncated TrkB comprise a region within the ectodomain of the truncated TrkB. In some instances, the ectodomain of the truncated TrkB comprises the leucine-rich repeat regions and the Ig-like domain that is involved in ligand interaction. In some instances, the epitope region within the ectodomain of the truncated TrkB comprises one or more cysteine residues. In some instances, the epitope region within the ectodomain of the truncated TrkB comprises one or more cysteine residues that is capable of forming disulfide bond. In some embodiments, the antibody or binding fragment thereof recognizes one or more of the epitopes that comprises one or more of the cysteine residues.

In some embodiments, the polypeptide antagonist comprises an antibody or binding fragment thereof that recognizes one or more of the epitopes of the truncated TrkC or truncated TrkB. In some instances, the polypeptide antagonist comprises an antibody or binding fragment thereof that recognizes one or more of the epitopes from the ectodomain of either truncated TrkC or truncated TrkB that comprises one or more of the cysteine residues.

In some embodiments, the polypeptide antagonist comprises an antibody or binding fragment thereof that recognizes one or more of the epitopes of the truncated TrkC or truncated TrkB but not one or more of the epitopes of the full-length TrkC or full-length TrkB. In some embodiments, the polypeptide antagonist comprises an antibody or binding fragment thereof that recognizes one or more of the epitopes from the ectodomain of the truncated TrkC or truncated TrkB but not one or more of the epitopes from the ectodomain of the full-length TrkC or full-length TrkB.

In some instances, the truncated TrkC is a non-catalytic truncated TrkC. As described elsewhere herein, the non-catalytic truncated TrkC protein comprises at least 80%, 85%, 90%, 95%, or 99% sequence identity to the amino acid sequence of SEQ ID NO: 10 or 113. In some instances, the truncated TrkC protein consists of the amino acid sequence of SEQ ID NO: 10 or 113. In some instances, the truncated TrkC is TrkC.T1.

In some embodiments, the truncated TrkB is a non-catalytic truncated TrkB. As described elsewhere herein, the non-catalytic truncated TrkB protein comprises at least 80%, 85%, 90%, 95%, or 99% sequence identity to an amino acid sequence selected from SEQ ID NOs: 11-12 and 121-123. In some instances, the truncated TrkB protein consists of an amino acid sequence selected from SEQ ID NOs: 11-12 and 121-123. In some instances, the truncated TrkB is TrkB.T1.

As used herein, the term “antibody” is used in the broadest sense and covers fully assembled antibodies, antibody fragments that bind antigen (e.g., Fab, F(ab′)₂, Fv, single chain antibodies such as single-chain variable fragment (scFv), diabodies, minibodies, single-domain antibodies (sdAbs) or nanobodies, antibody chimeras, hybrid antibodies, bispecific antibodies, humanized antibodies, and the like), and recombinant peptides comprising the forgoing.

As used herein, the terms “monoclonal antibody” and “mAb” as used herein refer to an antibody obtained from a substantially homogeneous population of antibodies, i.e., the individual antibodies comprising the population are identical except for possible naturally occurring mutations that are present in minor amounts.

Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

As used herein, the term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a 13-pleated-sheet configuration, connected by three CDRs, which form loops connecting, and in some cases forming part of, the f3-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

As used herein, the term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 5056 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody. In some embodiments, the portion of an intact antibody is an antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species assigned to one of two clearly distinct types, called kappa (x) and lambda (X), based on the amino acid sequences of their constant domains.

Depending on the amino acid sequence of the constant domain of their heavy chains, immunoglobulins are assigned to different classes. There are five major classes of human immunoglobulins: IgA, IgD, IgE, IgG, and IgM, and several of these are further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

### Methods of Antagonist Delivery

In some embodiments, the pharmaceutical composition comprises a vector in which the vector comprises one or more of the nucleic acid polymer described herein or comprise nucleic acid sequence that encodes a polypeptide described herein. In some embodiments, the nucleic acid polymer comprises at least 80%, 85%, 90%, 95%, or 99% sequence identity to a nucleic acid sequence selected from SEQ ID NOs: 1-5 and 114-119. In some embodiments, the polypeptide is an antibody or binding fragment thereof. In some embodiments, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, single-domain antibody (sdAb), or camelid antibody or binding fragment thereof. In some embodiments, the vector is a viral vector.

In some embodiments, the viral vector is obtained from any virus, such as a DNA or an RNA virus. In some embodiments, a DNA virus is a single-stranded (ss) DNA virus, a double-stranded (ds) DNA virus, or a DNA virus that contains both ss and ds DNA regions. In some embodiments, an RNA virus is a single-stranded (ss) RNA virus or a double-stranded (ds) RNA virus. In some embodiments, a ssRNA virus is further classified into a positive-sense RNA virus or a negative-sense RNA virus.

In some instances, the viral vector is obtained from a dsDNA virus of the family: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, and Tectiviridae.

In some cases, the viral vector is obtained from a ssDNA virus of the family: Anelloviridae, Bacillariodnaviridae, Bidnaviridae, Circoviridae, Geminiviridae, Inoviridae, Microviridae, Nanoviridae, Parvoviridae, and Spiraviridae.

In some embodiments, the viral vector is obtained from a DNA virus that contains both ss and ds DNA regions. In some cases, the DNA virus is from the group pleolipoviruses. In some cases, the pleolipoviruses include Haloarcula hispanica μleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

In some cases, the viral vector is obtained from a dsRNA virus of the family: Birnaviridae, Chrysoviridae, Cystoviridae, Endornaviridae, Hypoviridae, Megavirnaviridae, Partitiviridae, Picobirnaviridae, Reoviridae, Rotavirus and Totiviridae.

In some instances, the viral vector is obtained from a positive-sense ssRNA virus of the family: Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Secoviridae, Togaviridae, Tombusviridae, Tymoviridae, and Virgaviridae.

In some cases, the viral vector is obtained from a negative-sense ssRNA virus of the family: Bornaviridae, Filoviridae, Paramyxoviridae, Rhabdoviridae, Nyamiviridae, Arenaviridae, Bunyaviridae, Ophioviridae, and Orthomyxoviridae.

In some instances, the viral vector is obtained from oncolytic DNA viruses that comprise capsid symmetry that is isocahedral or complex. In some cases, icosahedral oncolytic DNA viruses are naked or comprise an envelope. Exemplary families of oncolytic DNA viruses include the Adenoviridae (for example, Adenovirus, having a genome size of 36-38 kb), Herpesviridae (for example, HSV1, having a genome size of 120-200 kb) and Poxviridae (for example, Vaccinia virus and myxoma virus, having a genome size of 130-280 kb).

In some cases, the viral vector is obtained from oncolytic RNA viruses include those having icosahedral or helical capsid symmetry. In some cases, icosahedral oncolytic viruses are naked without envelope and include Reoviridae (for example, Reovirus, having a genome of 22-27 kb) and Picornaviridae (for example, Poliovirus, having a genome size of 7.2-8.4 kb). In other cases, helical oncolytic RNA viruses are enveloped and include Rhabdoviridae (for example, VSV, having genome size of 13-16 kb) and Paramyxoviridae (for example MV and NDV, having genome sizes of 16-20 kb).

Exemplary viral vectors include, but are not limited to, retroviral vectors, lentiviral vectors, adenoviral vectors, adeno-associated viral vectors, alphaviral vectors, herpes simplex virus vectors, vaccinia viral vectors, or chimeric viral vectors. In some embodiments, the viral vector is a lentiviral vector. In some embodiments, the lentiviral vector is pLKO.1 vector.

In some instances, a virus comprising one or more of the nucleic acid polymers or polypeptides described herein are generated using methods well known in the art. In some instances, the methods involve one or more transfection steps and one or more infection steps. In some instances, a cell line such as a mammalian cell line, an insect cell line, or a plant cell line is infected with a virus to produce one or more viruses. Exemplary mammalian cell lines include: 293A cell line, 293FT cell line, 293F cells, 293 H cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, 3T6, A549, A9, AtT-20, BALB/3T3, BHK-21, BHL-100, BT, Caco-2, Chang, Clone 9, Clone M-3, COS-1, COS-3, COS-7, CRFK, CV-1, D-17, Daudi, GH1, GH3, H9, HaK, HCT-15, HEp-2, HL-60, HT-1080, HT-29, HUVEC, I-10, IM-9, JEG-2, Jensen, K-562, KB, KG-1, L2, LLC-WRC 256, McCoy, MCF7, VERO, WI-38, WISH, XC, or Y-1. Exemplary insect cell lines include Drosophila S2 cells, 519 cells, Sf21 cells, High Five™ cells, or expresSF+® cells. Exemplary plant cell lines include algae cells such as for example Phaeocystis pouchetii.

In some embodiments, the vector comprising one or more of the nucleic acid polymer or polypeptide described herein is delivered through electroporation, chemical method, microinjection, gene gun, impalefection, hydrodynamics-based delivery, continuous infusion, or sonication. In some embodiments, the chemical method is lipofection. In some cases, the method is infection, or adsorption or transcytosis.

In some embodiments, electroporation is a technique in which an electric field is applied to cells to increase the permeability of the cell membrane, allowing for the introduction of chemicals, drugs, or DNA into the cell.

In some embodiments, chemical method is a method of transfection that uses carrier molecules to overcome the cell-membrane barrier. In some instances, the chemical method is lipofection whereby genetic material is injected into a cell using liposomes.

In some embodiments, microinjection is the injection of genetic material into animal cells, tissues or embryos via a needle.

In some embodiments, gene gun is a device that injects cells with genetic information by shooting them with elemental particle of a heavy metal coated with plasmid DNA.

In some embodiments, impalefection is a method of gene delivery using nanomaterials.

In some embodiments, hydrodynamics-based delivery is the rapid injection of a relatively large volume of solution into a blood vessel to enhance the permeability to allow for the delivery of substance into cells. In some instances, the solution contains proteins, oligo nucleotides, DNA, RNA, or small molecules.

In some embodiments, continuous infusion is the uninterrupted administration of drugs, fluids or nutrients into a blood vessel.

In some embodiments, sonication is applying sound energy to agitate particles in a sample for purposes such as but not limited to disrupting or deactivating a biological material or fragmenting molecules of DNA.

In some embodiments, the nucleic acid polymer is delivered as an injection, such as an intramuscular, intrathecal, intravitreal, intraconjunctival intravenous or subcutaneous injection, without the need of a viral delivery method or non-viral delivery methods such as electroporation, chemical method, microinjection, gene gun, impalefection, hydrodynamics-based delivery, continuous infusion, or sonication. In some instances, the vector as described above is delivered as an injection, such as an intramuscular, intrathecal, intravitreal, intraconjunctival intravenous or subcutaneous injection, without the need of a viral delivery method or non-viral delivery methods such as electroporation, chemical method, microinjection, gene gun, impalefection, hydrodynamics-based delivery, continuous infusion, or sonication.

In some embodiments, the nucleic acid polymer and/or the vector described above further comprises a delivery vehicle. In some instances, the delivery vehicle comprises a lipid-based nanoparticle; a cationic cell penetrating peptide (CPP); or a linear or branched cationic polymer; or a bioconjugate, such as cholesterol, bile acid, lipid, peptide, polymer, protein, or an aptamer, which is conjugated to the nucleic acid polymer or polypeptide described herein for intracellular delivery. In some instances, additional delivery vehicles comprise glycopolymer, carbohydrate polymer, or lipid polymers such as cationic lipids or cationic lipid polymers.

### Diseases

Disclosed herein, in certain embodiments, include a method of treating a non-otic disease or condition associated with an elevated expression level or activity of truncated TrkC or truncated TrkB. In some instances, the method comprises administering to a patient having a non-otic disease or condition a therapeutic amount of a pharmaceutical composition described herein. In some instances, also described herein include a method of preventing a non-otic disease or condition associated with an elevated expression level or activity of truncated TrkC or truncated TrkB. In some instances, the method comprises administering to a patient having a non-otic disease or condition a therapeutic amount of a pharmaceutical composition described herein.

In some embodiments, the non-otic diseases or conditions comprise neurodegenerative disease or condition, or a symptomatic or pre-symptomatic condition with loss or alterations of synapses. In some instances, the non-otic diseases or conditions comprise neurodegenerative disorders. In some embodiments, the neurodegenerative disease or condition comprises polyglutamine expansion disorder, fragile X syndrome, fragile XE mental retardation, Friedreich's ataxia, myotonic dystrophy, spinocerebellar ataxia type 8, and spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, glaucoma, ischemia stroke, Krabbe disease, Lewy body dementia, multiple sclerosis, multiple system atrophy, Parkinson's disease, Pelizaeus-Merzbacher disease, Pick's disease, primary lateral sclerosis, progressive muscular atrophy, progressive bulbar palsy, pseudobulbar palsy, retinitis pigmentosa, Refsum's disease, Sandhoff disease, Schilder's disease, spinal cord injury (SCI), spinal muscular atrophy (SMA), Steele-Richardson-Olszewski disease, and Tabes dorsalis.

In some embodiments, the polyglutamine repeat disease comprises Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17).

In some embodiments, the non-otic disease or condition comprises amyotrophic lateral sclerosis (ALS), spinal muscular atrophy (SMA), glaucoma, spinal cord injury (SCI), or retinitis pigmentosa. In some embodiments, the non-otic disease or condition is amyotrophic lateral sclerosis (ALS). In some embodiments, the non-otic disease or condition is spinal muscular atrophy (SMA). In some embodiments, the non-otic disease or condition is glaucoma. In some embodiments, the non-otic disease or condition is spinal cord injury (SCI). In some embodiments, the non-otic disease or condition is retinitis pigmentosa.

In some instances, the non-otic diseases or conditions comprise psychiatric disorders. Exemplary psychiatric disorders comprise depression, psychosis, schizophrenia, narcolepsy, suicide tendency, synaptopathy or eating disorder (e.g., compulsive overeating or obesity).

### Pharmaceutical Composition/Formulations

In some embodiments, pharmaceutical compositions or formulations described herein are administered to a subject by multiple administration routes, including but not limited to, parenteral (e.g., intravenous, intrathecal, intravitreal, intraconjunctival, subcutaneous, intramuscular), oral, intranasal, buccal, topical, rectal, or transdermal administration routes. In some instances, the pharmaceutical composition describe herein is formulated for parenteral (e.g., intravenous, subcutaneous, intramuscular) administration. In other instances, the pharmaceutical composition describe herein is formulated for oral administration. In still other instances, the pharmaceutical composition describe herein is formulated for intranasal administration.

In some embodiments, pharmaceutical formulation described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, pharmaceutical formulations described herein include a carrier or carrier materials which include any commonly used excipients in pharmaceutics and are selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, pharmaceutical formulations include dispersing agents, and/or viscosity modulating agents which include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908°, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol has a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

In some embodiments, pharmaceutical formulations include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, pharmaceutical formulations also include one or more salts in an amount required to bring osmolality of the composition into an acceptable range. Such salts include those such as having sodium, potassium or ammonium cations and chloride, citrate, ascorbate, borate, phosphate, bicarbonate, sulfate, thiosulfate or bisulfite anions; suitable salts include sodium chloride, potassium chloride, sodium thio sulfate, sodium bisulfite and ammonium sulfate.

In some embodiments, pharmaceutical formulations further include diluent which are also used to stabilize compounds because they provide a more stable environment. Salts dissolved in buffered solutions (which also provide pH control or maintenance) are utilized as diluents in the art, including, but are not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some embodiments, pharmaceutical formulations include disintegration agents or disintegrants to facilitate the breakup or disintegration of a substance. The term “disintegrate” includes both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sor), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In some embodiments, pharmaceutical formulations include flavoring agents and/or sweeteners” such as for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

Lubricants and glidants also included in the pharmaceutical formulations described herein, for example, include those that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, CabOSil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants are included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Injectable Formulations

Formulations suitable for intramuscular, intrathecal, intravitreal, intraconjunctival subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain additives such as preserving, wetting, emulsifying, and dispensing agents. Prevention of the growth of microorganisms is ensured by various antibacterial and antifungal agents, such as parabens, chlorobutanol, phenol, sorbic acid, and the like. It also is desirable to include isotonic agents, such as sugars, sodium chloride, and the like. Prolonged absorption of the injectable pharmaceutical form is brought about by the use of agents delaying absorption, such as aluminum monostearate and gelatin.

For intravenous injections, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are generally known in the art.

In some instances, parenteral injections involve bolus injection or continuous infusion. Formulations for injection is presented in unit dosage form, e.g., in ampoules or in multi-dose containers, with an added preservative. The pharmaceutical composition described herein is in a form suitable for parenteral injection as a sterile suspensions, solutions or emulsions in oily or aqueous vehicles, and contains formulatory agents such as suspending, stabilizing and/or dispersing agents. Pharmaceutical formulations for parenteral administration include aqueous solutions of the active compounds in water-soluble form. Additionally, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions contain substances which increase the viscosity of the suspension, such as sodium carboxymethyl cellulose, sorbitol, or dextran. Optionally, the suspension also contains suitable stabilizers or agents which increase the solubility of the compounds to allow for the preparation of highly concentrated solutions. Alternatively, the active ingredient is in powder form for constitution with a suitable vehicle, e.g., sterile pyrogen-free water, before use.

Oral Formulations

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions are used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are added to the tablets or dragee coatings for identification or to characterize different combinations of active compound doses.

Solid dosage forms are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other instances, the pharmaceutical formulation is in the form of a powder. In still other instances, the pharmaceutical formulation is in the form of a tablet, including but not limited to, a fast-melt tablet. Additionally, pharmaceutical formulations described herein are administered as a single capsule or in multiple capsule dosage form. In some cases, the pharmaceutical formulation is administered in two, or three, or four, capsules or tablets.

In some embodiments, the pharmaceutical solid dosage forms include a composition described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000).

In some embodiments, suitable carriers for use in the solid dosage forms include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

In some embodiments, suitable filling agents for use in the solid dosage forms include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Binders impart cohesiveness to solid oral dosage form formulations: for powder filled capsule formulation, they aid in plug formation that are filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocer), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

Suitable lubricants or glidants for use in the solid dosage forms include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms include, for example, e.g., butylated hydroxytoluene (BHT), sodium ascorbate, and tocopherol.

Liquid formulation dosage forms for oral administration include aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystalline inhibitor.

In some embodiments, the aqueous suspensions and dispersions described herein remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension is re-suspended into a homogenous suspension by physical agitation lasting less than 1 minute. In another aspect, an aqueous suspension is re-suspended into a homogenous suspension by physical agitation lasting less than 45 seconds. In yet another aspect, an aqueous suspension is re-suspended into a homogenous suspension by physical agitation lasting less than 30 seconds. In still another embodiment, no agitation is necessary to maintain a homogeneous aqueous dispersion.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocer-E, Opadry YS, PrimaFlo, Benecel MP824, and Benecel MP843, methylcellulose polymers such as Methocer-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Plasticizers include polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compositions are formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media are also used. Furthermore, other methods such as roller compaction, extrusion/spheronization, coacervation, or nanoparticle coating are also used.

Intranasal Formulations

Intranasal formulations are known in the art and are described in, for example, U.S. Pat. Nos. 4,476,116 and 6,391,452. Formulations that include the compositions described herein, which are prepared according to the above described and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these are found in Remington: The Science and Practice of Pharmacy, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are also present. The nasal dosage form should be isotonic with nasal secretions.

For administration by inhalation described herein include aerosol, mist or powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the compound described herein and a suitable powder base such as lactose or starch.

### Dosing and Treatment Regimens

In some embodiments, the pharmaceutical compositions described herein are administered for therapeutic applications. In some embodiments, the pharmaceutical compositions described herein are also administered as a maintenance therapy, for example for a patient in remission. In some embodiments, the pharmaceutical composition is administered once per day, twice per day, three times per day or more. In some embodiments, the pharmaceutical composition is administered daily, every day, every alternate day, five days a week, once a week, every other week, two weeks per month, three weeks per month, once a month, twice a month, three times per month, or more. In some embodiments, the pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the compounds is given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, are reduced, as a function of the symptoms, to a level at which the improved disease, disorder or condition is retained. In some instances, patients, however, require intermittent treatment of a pharmaceutical composition described herein on a long-term basis upon any recurrence of symptoms.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, the pharmaceutical composition described herein is in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. Aqueous suspension compositions in some instances are packaged in single-dose non-redo sable containers. Alternatively, multiple-dose redo sable containers in some instances are used, in which case it is typical to include a preservative in the composition. By way of example only, formulations for parenteral injection are presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

According to another aspect of the invention, there is provided a method of selecting a subject for treatment, comprising determining if the subject has a disease induced by defective protein expression caused by the intron retention in gene transcripts, wherein the subject is selected for treatment upon positive confirmation; and optionally treating the subject.

### Diagnostic Methods

In certain embodiments, also included are methods of stratifying an individual having a non-otic disease or condition for treatment with a pharmaceutical composition described herein and methods of optimizing the therapy of an individual receiving a pharmaceutical composition described herein for treatment of a non-otic disease or condition. In some instances, disclosed herein is a method of stratifying an individual having a non-otic disease or condition for treatment with a pharmaceutical composition described herein, comprising: determining the expression level of a truncated TrkC or truncated TrkB; and administering to the individual a therapeutically effective amount of the pharmaceutical composition if there is an elevated expression level of the truncated TrkC or truncated TrkB. In some instances, also disclosed herein is a method of optimizing the therapy of an individual receiving a pharmaceutical composition described herein for treatment of a non-otic disease or condition, comprising: determining the expression level of a truncated TrkC or truncated TrkB; and modifying, discontinuing, or continuing the treatment based on the expression level of the truncated TrkC or truncated TrkB.

Methods for determining the expression and/or activity of truncated TrkC and/or truncated TrkB are well known in the art. In some embodiments, the expression levels are measured at either nucleic acid level or protein level, and by methods such as RT-PCR, Qt-PCR, micro array, Northern blot, ELISA, radioimmunoassay (RIA), electrochemiluminescence (ECL), Western blot, multiplexing technologies, or other similar methods. In some embodiments, activities of the truncated TrkC and/or truncated TrkB are measured by methods such as co-immunoprecipitation, fluorescence spectroscopy, fluorescence resonance energy transfer (FRET), isothermal titration calorimetry (ITC), dynamic light scattering (DLS), surface plasmon resonance (SPR), or other similar methods.

In some embodiments, the expression of truncated TrkC and/or truncated TrkB is determined at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA is utilized for the purification of RNA (see, e.g., Ausubel et al., ed. (1987-1999) Current Protocols in Molecular Biology (John Wiley & Sons, New York). Additionally, large numbers of tissue samples are readily processed using techniques well known to those of skill in the art, such as, for example, the single-step RNA isolation process disclosed in U.S. Pat. No. 4,843,155.

Thus, in some embodiments, the detection of a biomarker such as truncated TrkC or truncated TrkB is assayed at the nucleic acid level using nucleic acid probes. The term “nucleic acid probe” refers to any molecule that is capable of selectively binding to a specifically intended target nucleic acid molecule, for example, a nucleotide transcript. Probes are synthesized by one of skill in the art, or derived from appropriate biological preparations. Probes are specifically designed to be labeled, for example, with a radioactive label, a fluorescent label, an enzyme, a chemiluminescent tag, a colorimetric tag, or other labels or tags that are discussed above or that are known in the art. Examples of molecules that are utilized as probes include, but are not limited to, RNA and DNA.

For example, isolated mRNA are used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that hybridize to the mRNA encoded by the gene being detected. The nucleic acid probe comprises of, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR (see, for example, U.S. Pat. No. 4,683,202), ligase chain reaction (Barany (1991) Proc. Natl. Acad. Sci. USA 88:189 193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan System).

Expression levels of an RNA of interest are monitored using a membrane blot (such as used in hybridization analysis such as Northern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of expression also comprises using nucleic acid probes in solution.

In some embodiments, microarrays are used to determine expression of one or more biomarkers of truncated TrkC and/or truncated TrkB. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992, 6,020,135, 6,033,860, 6,344,316, and U.S. Pat. Application 20120208706. High-density oligonucleotide arrays are particularly useful for determining the gene expression profile for a large number of RNA's in a sample. Exemplary microarray chips include FoundationOne and FoundationOne Heme from Foundation Medicine, Inc; GeneChip® Human Genome U133 Plus 2.0 array from Affymetrix; and Human DiscoveryMAP® 250+ v. 2.0 from Myraid RBM.

Techniques for the synthesis of these arrays using mechanical synthesis methods are described in, e.g., U.S. Pat. No. 5,384,261. In some embodiments, an array is fabricated on a surface of virtually any shape or even a multiplicity of surfaces. In some embodiments, an array is a planar array surface. In some embodiments, arrays include peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. In some embodiments, arrays are packaged in such a manner as to allow for diagnostics or other manipulation of an all-inclusive device.

Any means for specifically quantifying a biomarker such as for example truncated TrkC and/or truncated TrkB in the biological sample of a candidate subject is contemplated. Thus, in some embodiments, expression level of a biomarker protein of interest in a biological sample is detected by means of a binding protein capable of interacting specifically with that biomarker protein or a biologically active variant thereof. In some embodiments, labeled antibodies, binding portions thereof, or other binding partners are used. The word “label” when used herein refers to a detectable compound or composition that is conjugated directly or indirectly to the antibody so as to generate a “labeled” antibody. In some embodiments, the label is detectable by itself (e.g., radioisotope labels or fluorescent labels) or, in the case of an enzymatic label, catalyzes chemical alteration of a substrate compound or composition that is detectable.

The antibodies for detection of a biomarker protein are either monoclonal or polyclonal in origin, or are synthetically or recombinantly produced. The amount of complexed protein, for example, the amount of biomarker protein associated with the binding protein, for example, an antibody that specifically binds to the biomarker protein, is determined using standard protein detection methodologies known to those of skill in the art. A detailed review of immunological assay design, theory and protocols are found in numerous texts in the art (see, for example, Ausubel et al., eds. (1995) Current Protocols in Molecular Biology) (Greene Publishing and Wiley-Interscience, NY)); Coligan et al., eds. (1994) Current Protocols in Immunology (John Wiley & Sons, Inc., New York, N.Y.).

The choice of marker used to label the antibodies will vary depending upon the application. However, the choice of the marker is readily determinable to one skilled in the art. These labeled antibodies are used in immunoassays as well as in histological applications to detect the presence of any biomarker of interest. The labeled antibodies are either polyclonal or monoclonal. Further, the antibodies for use in detecting a protein of interest are labeled with a radioactive atom, an enzyme, a chromophoric or fluorescent moiety, or a colorimetric tag as described elsewhere herein. The choice of tagging label also will depend on the detection limitations desired. Enzyme assays (ELISAs) typically allow detection of a colored product formed by interaction of the enzyme-tagged complex with an enzyme substrate. Radionuclides that serve as detectable labels include, for example, 1-131, 1-123, 1-125, Y-90, Re-188, Re-186, At-211, Cu-67, Bi-212, and Pd-109. Examples of enzymes that serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The antibodies are conjugated to these labels by methods known in the art. For example, enzymes and chromophoric molecules are conjugated to the antibodies by means of coupling agents, such as dialdehydes, carbodiimides, dimaleimides, and the like. Alternatively, conjugation occurs through a ligand-receptor pair. Examples of suitable ligand-receptor pairs are biotin-avidin or biotin-streptavidin, and antibody-antigen.

In certain embodiments, expression of one or more biomarkers of interest within a biological sample, for example, a cell sample, is determined by radioimmunoassays or enzyme-linked immunoassays (ELISAs), competitive binding enzyme-linked immunoassays, dot blot (see, for example, Promega Protocols and Applications Guide, Promega Corporation (1991), Western blot (see, for example, Sambrook et al. (1989) Molecular Cloning, A Laboratory Manual, Vol. 3, Chapter 18 (Cold Spring Harbor Laboratory Press, Plainview, N.Y.), chromatography such as high performance liquid chromatography (HPLC), or other assays known in the art. Thus, the detection assays involve steps such as, but not limited to, immunoblotting, immunodiffusion, immunoelectrophoresis, or immunoprecipitation.

In some embodiments, the activities of the truncated TrkC and/or truncated TrkB are measured by methods such as co-immunoprecipitation, fluorescence spectroscopy, fluorescence resonance energy transfer (FRET), isothermal titration calorimetry (ITC), dynamic light scattering (DLS), surface plasmon resonance (SPR), or other similar methods.

In some embodiments, truncated trkC and/or truncated TrkB are measured using in vivo imaging techniques, a such as PET, OCT, MRI; combined with labeled or unlabeled antibodies, peptides, small molecules or related binding agents of truncated TrkC or truncated TrkB.

Samples

In certain embodiments, one or more of the methods disclosed herein comprise a sample. In some embodiments, the sample is a cell sample or a tissue sample. In some instances, the sample is a cell sample. In additional instances, the sample is a tissue sample. In some embodiments, the sample for use with the methods described herein is obtained from cells or tissues of an animal. In some instances, the animal is a human, a non-human primate, or a rodent.

In some embodiments, the cell or tissue sample comprises neurons or glial cells (or neuroglia). In some instances, neurons comprise sensory neurons, interneurons, or motor neurons. In some embodiments, glial cells are non-neuronal cells that maintain homeostasis, form myelin, and provide support and protection for neurons in the central and peripheral nervous systems. In some embodiments, glial cells further comprise astrocytes, microglia, and Müller glia. Astrocytes or astroglia are star-shaped glial cells located in the brain and spinal cord. Microglia are macrophages that comprises phagocytosis function and protect neurons of the central nervous system. Müller glia are a type of retinal glial cells that maintain the stability of the retinal extracellular environment by regulation of K+ levels, uptake of neurotransmitters, removal of debris, storage of glycogen, electrical insulation of receptors and other neurons, and mechanical support of the neural retina. In some embodiments, the cell or tissue sample comprises astrocytes. In some embodiments, the cell or tissue sample comprises microglia. In some embodiments, the cell or tissue sample comprises Müller glia. In some embodiments, the cell or tissue sample comprises neurons such as sensory neurons, interneurons, or motor neurons.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include one or more of the pharmaceutical compositions described herein comprising an antagonist of a truncated TrkC or truncated TrkB isoform and an excipient and/or delivery vehicle. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

These examples are provided for illustrative purposes only and not to limit the scope of the claims provided herein.

### Example 1

### Deleterious Mechanism of TrkC.T1 Receptors in Neurodegeneration

TrkC.T1 activates its own signal transduction cascade which is different from TrkC-FL. TrkC.T1 activated Tamalin and Rho kinase in an NT-3-dependent manner in cultured cells. Importantly, it is shown that in vivo TrkC.T1 activates neurotoxic mechanisms. In a neurodegenerative disease and loss of synapse of the retina, glaucoma, TrkC.T1 is expressed early de novo and at high levels in glia/Müller cells. TrkC.T1 regulates production of neurotoxic TNF-α, which in turn kills retinal neurons. To validate TrkC.T1 as a therapeutic target, a “TrkC.T1 knockout mouse” was generated where the mRNA splice site is mutated. The mouse makes normal TrkC-FL but does not splice it to make TrkC.T1 mRNA. The TrkC.T1^{-/-} mouse has no detectable phenotype. However, the TrkC.T1^{-/-} mouse is resistant to neurodegeneration when glaucoma is induced. The TrkC.T1^{-/-} was resistant but less than the TrkC.T1^{-/-}, suggesting a gene dosage effect. Resistance to degeneration is associated with a reduced increase in TNF-α during disease.

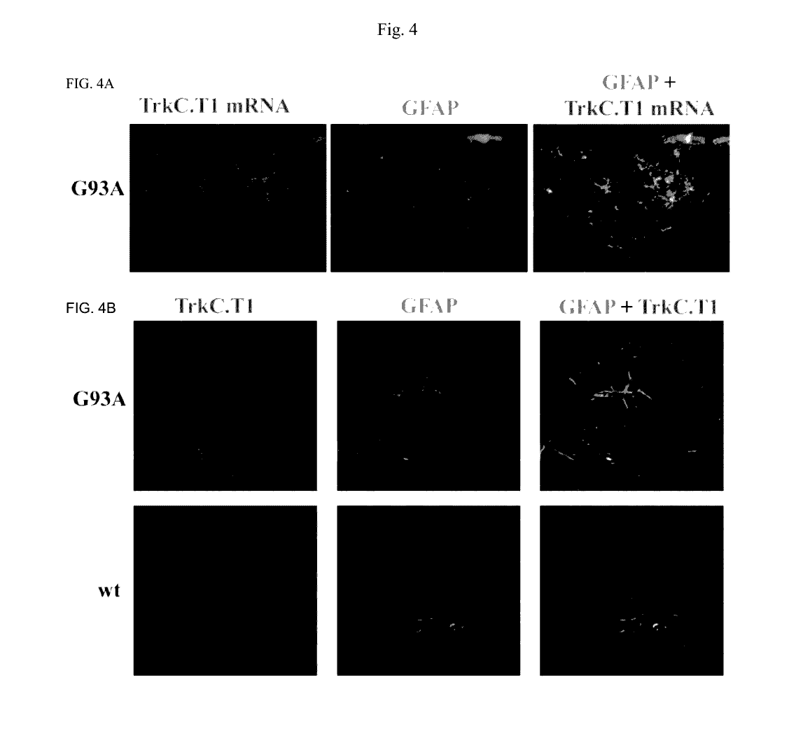

In some instances, it is also shown that in glaucoma pharmacological inhibitors of TrkC prevent elevation of TNF-α, and delayed neuronal death and disease progression in vivo. In some cases, TrkC.T1 mRNA and protein are up-regulated in human ALS and animal models of ALS. TrkC.T1 mRNA (FIG. 4A) and protein (FIG. 4B) are expressed de novo in the spinal cord in the G93A mouse model of ALS, but are not present in wild type healthy spinal cords. The mRNA and protein co-localize with GFAP, indicating expression in spinal cord astrocytes.

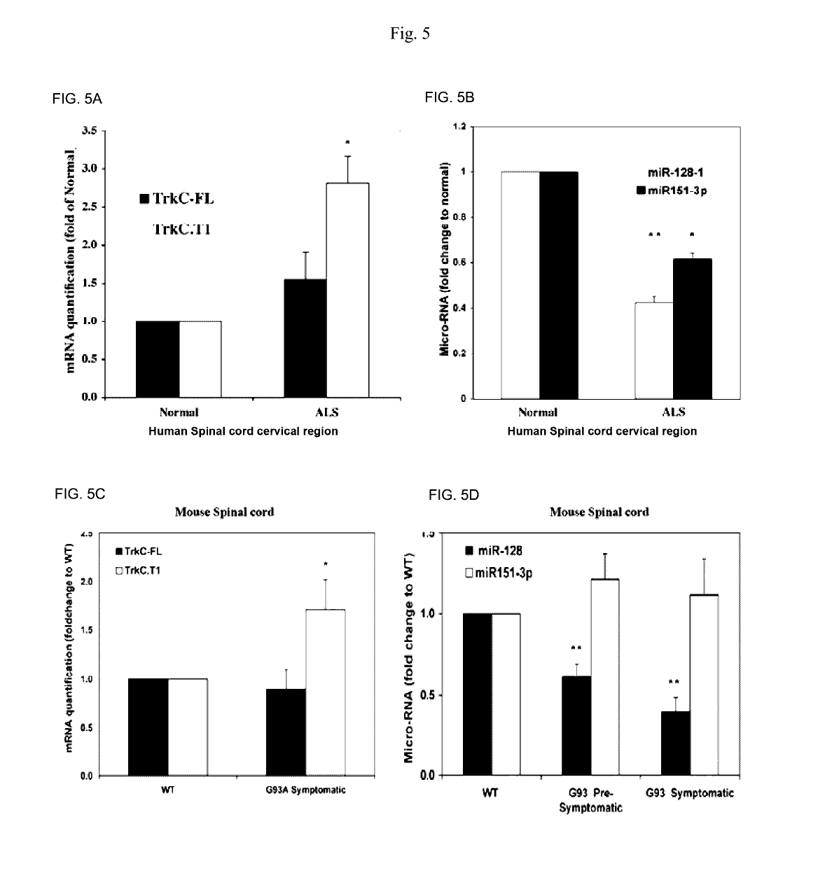

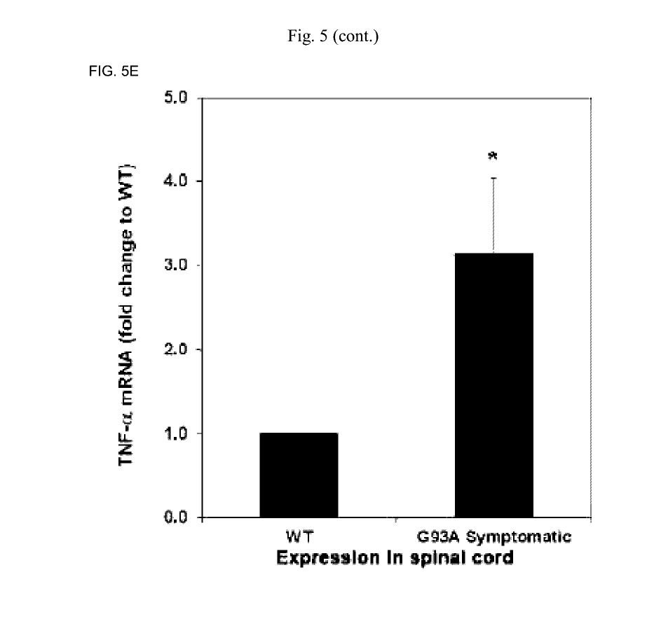

In some cases, it was found that TrkC.T1 mRNA and protein were up-regulated in spinal cords obtained from human donors with sporadic ALS (SALS), compared versus non-ALS control (FIG. 5A). Frozen spinal cord tissue from patients with confirmed non-SOD1 ALS (age range, 39-73) and non-ALS control (age range, 38-76) were studied. Elevated TrkC.T1 mRNA in human SALS is due to the reduced levels of miR128 (a microRNA that destabilizes TrkC.T1 mRNA) (FIG. 5B). In human SALS versus non-ALS control there was also a reduction in miR-151-3p (a microRNA disrupter of TrkC-FL) and this resulted in a small and non-significant elevation of TrkC-FL mRNA. In the mouse model of ALS the increase in TrkC.T1 mRNA in the spinal cord (FIG. 5C) also correlates with a decrease in miR128 levels (FIG. 5D) and an increase in TNF-α mRNA (FIG. 5E).

Upregulation of Truncated TrkC.T1 Receptor Isoform in Glaucoma Lead to Increased TNF-α Production

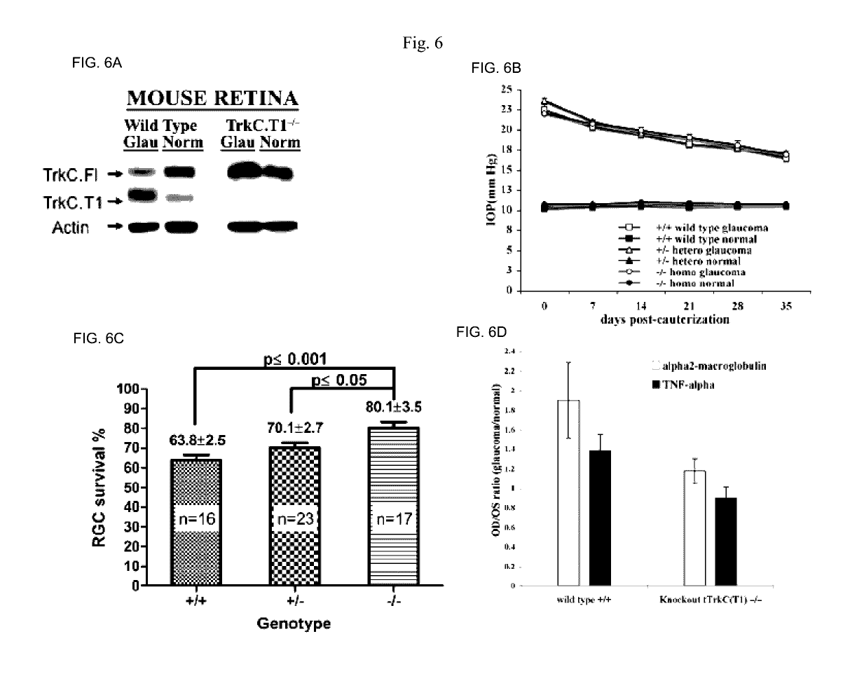

In normal wild type retinas, TrkC.FL protein and TrkC.T1 protein were expressed at relative low levels (FIG. 6A). After 14 days, glaucomatous retinas from wild type mice showed increased TrkC.T1 protein expression, 1.8±0.3-fold, compared to retinas without glaucoma (p<0.05, when the data was standardized to actin control). In contrast, the levels of TrkC.FL showed relatively no changes in glaucomatous retinas compared to retinas without glaucoma when the data was standardized to actin control (FIG. 6A).

In TrkC.T1^{-/-} retinas the TrkC.T1 protein was not detected, whether or not the retinas had glaucoma. However, in glaucomatous TrkC.T1^{-/-} retinas TrkC.FL expression was elevated 1.3±0.1-fold compared to the expression level of TrkC.FL in normal TrkC.T1^{-/-} retinas. After 14 days of glaucoma in wild type mice, the expression level of TrkC.T1 was elevated relative to a wild type mice without glaucoma. In the same wild type mice, the expression of TrkC.FL remained relatively unchanged. In the retinas of TrkC.T1^{-/-} mice, the expression of TrkC.T1 was undetected and the expression level of TrkC.FL remained relatively unchanged.

In a model of glaucoma, the normal TOP of naive eyes (left eye, OS) was the same irrespective of TrkC.T1 genotype. The cauterized eyes (right eye, OD) in wild type (n=16), TrkC.T1^{-/-} heterozygous mice (n=24), or TrkC.T1^{-/-} homozygous mice (n=20) experienced a comparable elevation of TOP over the 5 week term (FIG. 6B). Thus, the absence of TrkC.T1 did not prevent TOP elevation in this model of glaucoma.

To investigate whether lack of TrkC.T1 affected RGC development, RGC numbers in KO and WT mice with normal TOP were quantified. Retinas did not show any differences in RGC numbers irrespective of TrkC.T1 genotype. In additional cases, similar RGC numbers were quantified for KO and WT mice at ages 3, 6 and 12 months. In some instances, the RGCs of TrkC.T1 KO mice appeared to go through normal developmental proliferation, pruning, differentiation, maturation, and ageing.

TrkC(T1)^{-/-} Mice are Resistant to Glaucomatous RGC Death

In some instances, whether the deletion of TrkC.T1 correlates to an impact on RGCs during glaucoma was investigated. Labeled RGCs were counted after 5 weeks of elevated TOP, and were compared versus the contralateral nave eye.

The wild type group (n=16) had 64% RGCs labeled (or 36% loss), the heterozygous TrkC.T1^{+/-} group (n=23) had ˜70% RGCs labeled (or 30% loss), and the homozygous TrkC.T1^{-/-} group (n=17) had ˜80% RGCs labeled (or 20% loss). The TrkC.T1^{-/-} group exhibits a significant difference from wild type (p<0.001) and from TrkC.T1^{+/-} (p<0.05) (FIG. 6C). Therefore, the TrkC.T1^{-/-} mice exhibited a relative resistance to glaucomatous RGC death. The difference in RGCs loss between wild type mice with glaucoma (36%) and KO mice with glaucoma (20%) corresponded to about half of the total RGC damage. In some instances, this resistance was observed in spite of constant stress due to high TOP over the 5-week period.

The data was further analyzed by segregating the data according to age. The resistance to glaucomatous RGC death in TrkC.T1^{-/-} mice was independent of age. Young, adult, and aged TrkC.T1^{-/-} mice (3 months, 6 months, and 12 months) showed no statistically significant differences in their resistance to glaucomatous RGC death.

A multivariate analysis of total TOP over the time period, as well as age indicated that the only explanatory variable was TrkC.T1 genotype. However, in some instances the aged group, regardless of genotype, also had a trend to being more resistant to glaucomatous RGC death than the younger group.

TrkC.T1 Regulates Expression of α2m and TNF-α in Glaucomatous Retinas

In some instances, the expression of TrkC.T1 was deleterious for RGCs. In some cases, in glaucomatous eyes TNF-α and α2m was associated with RGC death. Hence, a study was carried out to determine whether a correlation exist between the expression of TrkC.T1 and the production of these neurotoxic factors.

The α2m and TNF-α protein in retinal samples from homozygous TrkC.T1^{-/-} mice or control wild type mice ages 3-5 months were quantified. Densitometric quantification and analyses of the western blot data compared protein expression in day 14 glaucomatous eyes versus contralateral normal eyes (n=5 mice/group±sem; standardized versus β-actin loading control) (FIG. 6D).

In TrkC.T1^{-/-} mice glaucoma did not elevate α2m (1.2±0.13 glaucoma/normal eye ratio) or TNF-α (0.9±0.12 glaucoma/normal eye ratio). In contrast, the wild type mice with glaucoma showed elevated α2m expression (1.9±0.38 glaucoma/normal ratio) and elevated TNF-α expression (1.4±0.17 glaucoma/normal ratio).

In some instances, these data suggested that expression of TrkC.T1, which was induced in wild type mice with experimental glaucoma, was associated with the up-regulation of neurotoxic factors TNF-α and α2m.

Development of Selective Inhibitors of TrkC.T1

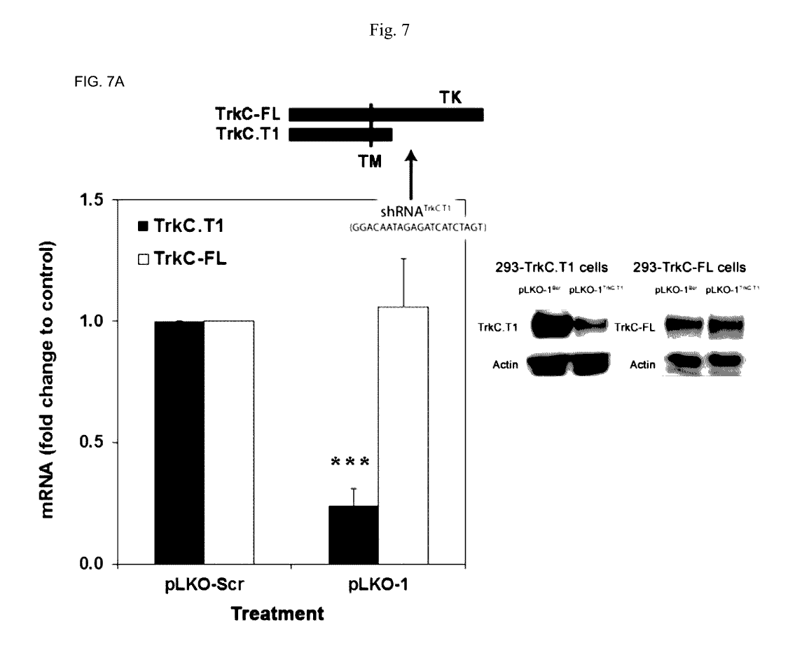

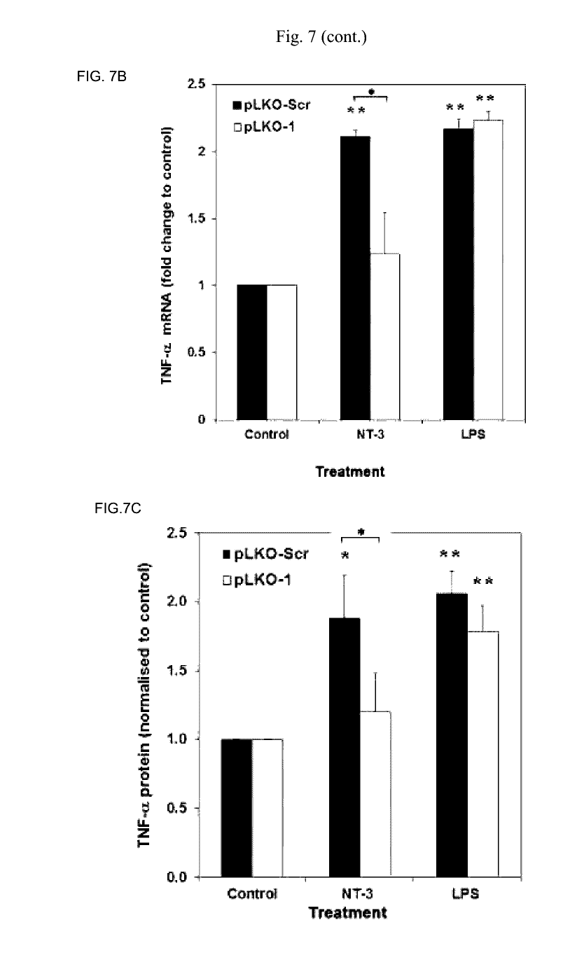

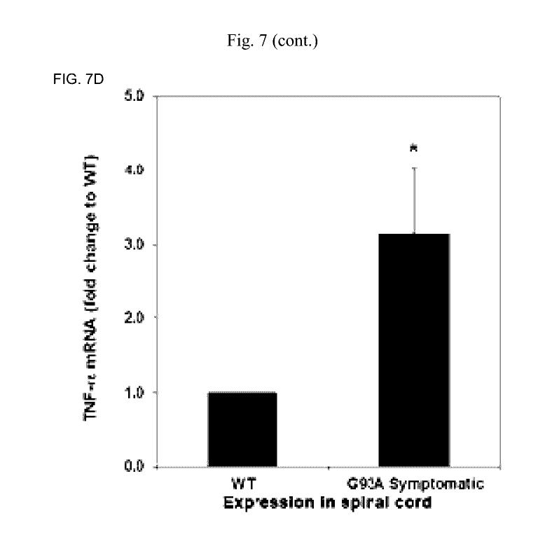

A short hairpin RNA (shRNA) specifically targeting a unique 3′ sequence of the TrkC.T1 mRNA was designed. The TrkC.T1-targeting shRNA sequence (GGACAATAGAGATCATCTAGT) (SEQ ID NO: 1), or a scrambled control (CCTAAGGTTAAGTCGCCCTCG) (SEQ ID NO: 8), were cloned into a pLKO.1 lentiviral shRNA-expression vector. pLKO.1^{scrambled} and pLKO.1^{TrkC-T1} lentivirus were purified and tested by infection of HEK293-TrkC.T1 or HEK293-TrkC-FL (cells transfected with TrkC.T1 or TrkC-FL cDNAs). Infection with PLKO.1^{TrkC.T1} reduced TrkC.T1 mRNA without affecting TrkC-FL mRNA, whereas infection with control virus PLKO.1^{Scrambled} had no effect on either TrkC.T1 or TrkC-FL mRNA (FIG. 7A). The data was verified studying protein expression in lysates of the same cells (FIG. 7A, inset). In multiple experiments TrkC.T1 protein expression in culture was reduced by ˜80% to 97%. To assess whether reduction of TrkC.T1 mRNA and protein had a biological impact, production of TNF-α was used as a functional endpoint for TrkC.T1 activity.

In rMC-1 cells treatment with NT-3 (10 nM) or LPS (1 μg/ml) for 6 hrs increases TNF-α mRNA (FIG. 7B) and protein (FIG. 7C). Infection with PLKO.1^{TrkC.T1} reduced NT-3 induced TNF-α (p=0.36 versus uninfected cells not treated with NT-3), but did not reduce LPS induced TNF-α. Infection with control PLKO.1^{Scrambled} did not alter the induction of TNF-α by NT-3 or LPS treatment. Together, the data indicate that TrkC.T1 regulates TNF-α in an NT-3 dependent manner. Given that TNF-α is up-regulated to toxic levels during ALS progression (FIG. 7D), the up-regulated TrkC.T1 in astrocytes during ALS in some instances play a role in TNF-α toxicity in vivo.

### Example 2

Retinitis pigmentosa (RP) is an inherited degenerative eye disease characterized by progressive apoptosis of photoreceptors and dysfunction of the retinal pigmented epithelium (RPE) that ultimately leads to largely irreversible loss of vision. Extremely heterogeneous in nature, degeneration of photoreceptors and RPE is triggered by one of more than 200 gene mutations. Even though over 50 genes have been identified, the etiology from gene mutation to photoreceptor dystrophy remains unknown.

The RHOP347S (Rhodopsin mutant, RHOP) transgenic mouse model of RP carries a mutated form of the human Rhodopsin gene in addition to the two normal mouse copies. The Rho gene codes for the long wavelength sensitive opsin found in rod receptor outer segments. In some instances, six different missense mutations affecting the proline-347 residue near the carboxyl terminus of the Rhodopsin protein have been identified among patients with RP. Proline-347 mutants show an abnormal extracellular accumulation of rhodopsin vesicles due to aberrant transport of rhodopsin from the inner to the outer segments that lead to photoreceptor cell death because of failure to renew outer segments at a normal rate. In rhodopsin mutant mice, cellular signaling pathways have not been identified as being involved in the mechanism underlying photoreceptor degeneration with the exception of the unfolded protein response.

The tropomyosin-related tyrosine kinase (Trk) receptors (TrkA, TrkB and TrkC) are receptor tyrosine kinases initially described as a family of growth factor receptors required for neuronal survival via intracellular signaling cascades. They have been since been shown to influence many aspects of neuronal development and function, including differentiation, axonal growth and synaptogenesis. In the retina, Trk receptors regulate the maintenance of the retinal structure, neuronal life and death, the health of pigmented epithelium, and glial activation. In addition to the full-length form receptor tyrosine kinases, the TrkC locus is generated by alternative splicing truncated receptor isoforms such TrkC.T1, which lacks the intracellular kinase domain and has a short intracellular domain. It has been contemplated that TrkC.T1 affects development. In some instances, the main function attributed to TrkC.T1 was the inhibition of the kinase-active receptor isoform, which is achieved by acting as a dominant receptor of TrkC or by sequestering NT-3 and does not require TrkC.T1 signal.

### Methods

### Cell Line

HEK293 cells were transfected with human full-length TrkC cDNA (293-TrkC-FL) or with rat TrkC.T1 cDNA (293-TrkC.T1). The cells are stably transfected subclones that express high levels of TrkC-FL or TrkC.T1 receptors and are respectively grown under drug selection (0.5 mg/ml G418 or 1 mg/ml Puromycin).

### RNAi Knockdown of TrkC.T1

A short hairpin RNA (shRNA) specifically targeting a unique 3′ sequence of the TrkC.T1 mRNA was designed using the DSIR algorithm. The TrkC.T1-targeting shRNA sequence GGACAATAGAGATCATCTAGT (SEQ ID NO: 1), or a scrambled control sequence CCTAAGGTTAAGTCGCCCTCG (SEQ ID NO: 8), were cloned into a pLKO.1 lentiviral shRNA expression vector. pLKO.1^{scrambled} and pLKO.1^{TrkC.T1}. rMC-1 cells were then transduced with lentiviral particles, and selected with 1 mg/ml puromycin. TrkC.T1-specific depletion was determined by real-time quantitative PCR and by Western blotting.

### Animal Models

All animal procedures respected the IACUC guidelines for use of animals in research, and to protocols approved by McGill University Animal Welfare Committees. All animals were housed 12 hour dark-light cycle with food and water ad libitum. A model of truncated TrkC.T1 knockout mouse developed in the laboratory was used, and the “RHOP347S” (RHOP) transgenic mouse (Rhodopsin mutant) model of retinitis pigmentosa was obtained from Dr. T. Li. Both mice models were consistently backcrossed onto a pure C57BL/6J (B6) genomic background. The RHOP347S mice were crossed with the TrkC.T1 knockout mice (TrkC.T1^{+/-}) or wild-type (WT) C57/BL6 mice to generate the following genotypes: WT, RHOP:TrkC.T1^{+/+}, RHOP:TrkC.T1^{+/-}, RHOP:TrkC.T1^{-/-}.

### Mice Genotypic Screening

PCR-based method for subsequent genotyping of the animals was used. Screening of TrkC.T1 was done using the same conditions as described in Bai et al., “In glaucoma the up-regulated truncated TrkC.T1 receptor isoform in glia causes increased TNF-α production, leading to retinal ganglion cell death,” Invest Ophthalmol Vis Sci 51: 6639-6651 (2010). Following primers were used: RM015 Rho F 5′ GGATTCTGTTTGACATGGGG 3′ (SEQ ID NO: 124) and RM016 Rho R 5′ TCCAGTCAGGACTCAAACCC 3′ (SEQ ID NO: 125) for RHOP mice screening, and forward 5′ ACCACAGTCCATGCCATCAC 3′ (SEQ ID NO: 126) and reverse 5′ TCCACCACCCTGTTGCTGTA 3′ (SEQ ID NO: 127) for GAPDH controls. PCR reactions were done using the “Extracta™ DNA Prep for PCR-Tissue” kit (Quanta Biosciences, MD, USA) according to the protocol provided by the manufacturer. All PCR conditions were performed as follows: 35 cycles at 96° C. for 2 minutes, 94° C. for 30 minutes, 58° C. for 30 minutes; 35 cycles at 72° C. for 40 minutes, 72° C. for 7 minutes.

### Intravitreal Injections

Intravitreal injections were performed as follows. Briefly, mice were anesthetized with isoflurane, delivered through a gas anesthetic mask. The drugs were delivered using a Hamilton syringe. Injections were done using a surgical microscope to visualize the Hamilton entry into the vitreous chamber and confirm delivery of the injected solution. After the injection, the syringe was left in place for 30 sec and slowly withdrawn from the eye to prevent reflux. Experimental right eyes were injected with the test agents and control left eyes serve as internal control.

### Drug Regiment-Pharmacological Inhibition

All intravitreal injections delivered 2 μl of the MAPK/ERK inhibitor PD98059 (10 mM stock) or the selective-TrkC antagonist 3F (1 mM stock). Control eyes were injected with 50% DMSO for PD98059 assays or 5% DMSO for the compound 3F assays. Intravitreal injections in wild-type or RHOP-mice were made at post-natal day 17. Experimental time points to measure ONL thinning were set at post-natal days 18, 22, 24 and 28. The end point for analyzing the effect of the compounds on the expression of p-Erk and p-Akt was at post-natal day 18 (24 hours after intravitreal injection).

### Drug Regiment-NT-3 Stimulation

All cells types were serum-starved for 1 hour. Afterwards, 293-TrkC-FL and non-infected 293-TrkC.T1 or 293-TrkC.T1 infected with lentiviral pLKO-1^{scrambled} or pLKO-1^{TrkC.T1} were treated with NT-3 4 nM for 10 min. Non-infected rMC-1 or cells infected with lentiviral pLKO.1^{scrambled} or pLKO.1^{TrkC.T1} were given the same NT-3 stimulation. Then, cells were collected and processed for Western blot analysis.

### Optical Coherence Tomography Imaging

A noninvasive prototype spectrometer-based FD-OCT system was used to acquire the retinal images. FD-OCT is a noninvasive method that allows time-kinetic studies in the same animal, with axial resolution in tissue nominally better than 4 μm and repeatability of the measurements from B-scans better than 1 μm. Data acquisition was performed using custom software written in C++ for rapid frame grabbing, processing, and display of two-dimensional images. Manual segmentations were used to measure the thicknesses of the mice retinas. Mice were anesthetized using isoflurane/O2 and placed on a platform, and the head was oriented to an angle at which the eye was properly aligned to the optical beam. The pupils were dilated with a topical solution (atropine sulfate 1%; Alcon, Fort Worth, Tex.). Refraction of light at the cornea was cancelled by placing over the eye a generic artificial tear gel. Alignment of the optical system to the rat retina required a few minutes and was followed by rapid acquisition of data (about 5 s/vol). During retinal scanning, three volumes were acquired in different sectors of the retina containing the ON head as landmark.

After processing, three B-scans were randomly selected from each volume. The retinal thickness measurements were performed with Matlab software using the saved data. In each B-scan, the thickness of the nerve fiber layer (NFL)—ganglion cell layer (GCL)—inner plexiform layer (IPL), hereafter referred to as NGI and outer nuclear layer (ONL) was measured at three adjacent points. Data are shown as average ONL thickness in μm±SEM (absolute values) in wild-type (WT) versus RHOP:TrkC.T1+/+ and RHOP:TrkC.T1+/−, or as average of ONL thickness in percentage (%)±SEM (relative values). For relative values, each vehicle-treated eye (OS, left eye) was established as 100% of the ONL thickness versus the drug-treated eye (OD, right eye).

### TrkC.T1 Detection by Fluorescence “In Situ” Hybridization

After enucleation, the eyes were immersed overnight in fixative composed of 4% paraformaldehyde (PFA) in PBS (pH 7.4) at 4° C., followed by cryoprotection by soaking in 30% sucrose overnight at 4° C. Eyes were embedded in OCT tissue TEK (VWR, QC, Canada) and frozen with dry ice. Then, cryostat sections were cut (20 μm thick) and mounted onto gelatincoated glass slides. Sections were re-fixed in 4% PFA, permeabilized with proteinase K for 12 min, and acetylated for 10 min. After, sections were prehybridated for 4 hours at room temperature. Hybridization with digoxigenin (DIG)-labeled TrkC.T1 antisense RNA or sense RNA (internal negative control) probes was performed overnight at 72° C. in a hybridization oven (Robbins Scientific, CA, USA). This was followed by non-stringent and stringent washes and incubation with anti-DIG-HRP (1:1000) overnight at 4° C. The amplification reaction was performed using the “TMR-conjugated TSA” kit (Perkin Elmer, MA, USA) according to the protocol provided by the manufacturer. Finally, sections were washed and cover-slipped using Vectashield mounting media with DAPI (Burlingame, Calif., USA). Positive staining was identified as red punctate dots in the section.

### Histology

The eyes were enucleated and immersed for 1 hour in fixative composed of 2% glutaraldehyde in PBS at room temperature. After, cornea was removed and the eye cups were placed in fresh 2% glutaraldehyde and incubated on nutator, overnight at room temperature. The eye cups were dehydrated in ascending grades of reagent alcohol and embedded into epon resin. Sections (800 nm thick) were cut on a LKB ultratome (LKB 2088 Ultrotome V, Sweden) and placed onto Superfrost Plus Slides (Fisher Scientific, ON, Canada). Slides were stained with 1% toluidine blue and mounted with Permount (Fisher Scientific, ON, Canada).

### Image Acquisition (Bright-Field Microscopy) and Data Analysis

Pictures were taken on a Leica DM LB2 microscope applying a 40× objective. Images were saved directly in TIF format and adjusted using Adobe Photoshop C58.0 for unbiased brightness and contrast.

For each experimental condition, 6 images were acquired from 3 sections cut from different areas of the retina (n=2 retinas per group) starting at 500 μm from the optic nerve head to avoid thickness variability due to the thinning of the retinal layers close to the boundaries of the optic nerve. The number of photoreceptors nuclei contained inside a rectangle of a fixed area (0.0156 mm²), drawn in the ONL, was counted using ImageJ software. Data are shown as the average number of cells per mm²±SEM.

### Immunohistochemistry

After enucleation, the eyes were immersed overnight in fixative composed of 4% PFA in PBS at 4° C., followed by cryoprotection by soaking in 30% sucrose overnight at 4° C. Eyes were frozen in O.C.T tissue TEK and cryostat sections were cut and mounted onto gelatin-coated glass slides. Sections (20 μm thick) were washed with phosphate-buffered saline (pH 7.4, PBS) and then, incubated in PBS containing 10% normal goat serum and/or 10% normal donkey serum, 0.3% Triton X-100 and 0.5% bovine serum albumin (BSA) for 2 hours. After, sections were incubated overnight at 4° C. with primary antibody. The sections were rinsed and incubated with secondary antibody (Table 1) for 1-2 hours at room temperature. Finally, sections were washed and cover-slipped using Permafluor (Thermo Fisher Scientific, Fremont, Calif., USA) or Vecta shiled mounting media with DAPI.

### Image Acquisition (Fluorescence Microscopy) and Data Analysis

Pictures were taken as Z-stacks of confocal optical sections using a Leica confocal microscope equipped with argon and helium neon lasers applying a 20× objective. Images were exported directly in TIE′ format and adjusted using Adobe Photoshop CS 8.0 for unbiased brightness and contrast.

For each experimental condition, a minimum of 6 images were acquired from 3 sections cut from different areas of the retina (n=3-4 retinas per group). The area of the profiles of the cells expressing p-Akt and p-Erk was measured using ImageJ software. For the “in situ” studies, an arbitrary rectangle of 129×97 pixels was drawn for each layer of the retina (GO, IPL, Mt and PhR) to measure the TrkC.T1 positive staining using imageJ. Data are shown as the average area (in pixels)±SEM in wild-type (WT) versus RHOP:TrkC.T1^{+/+} and RHOP:TrkC.T1^{+/-} or as normalized area values±SEM, giving WT mice the arbitrary value of 1.

### TUNEL Staining

Staining was performed using the DeadEnd Fluorometric TUNEL system (Promega.). Vehicle or compound 3F treated RHOP347S retinas in culture (n=6) were first fixed in 4% HA in PBS and kept at 4° C. overnight. Three quick washes in PBS-0.2% BSA were done the next day, followed by three 30-minute permeabilization steps using 2% Triton-X in PBS. Retinas were then incubated with 20 μg/mL Proteinase K in PBS for 15 minutes, briefly re-fixed in PFA for 30 minutes and washed again with PBS-0.2% BSA before being transferred into Eppendorf tubes. Samples were incubated with 50 μL of equilibration buffer for 20 minutes, then 254, of TdT reaction mixture for 2.5 hours at 37° C. The reaction was terminated using a 30-minute incubation of 2×SSC solution. The retinas were washed and mounted between two coverslips with the ganglion-cell layer facing up using Vectashield with DAN as this was easier to achieve given the natural curvature of the retina. The coverslipped samples were then flipped over and taped to microscope slides to have the photoreceptor-side facing up. For image acquisition, the retinas were divided into 4 quadrants, and 2 pictures with a 20× objective were taken in each area (one central and one peripheral) for a total of 8 images of the outer-nuclear layer (ONL) per retina. Total TUNEL-positive cells were counted in each image semi-automatically (ImageJ) by 3 independent blinded examiners. Wild-type retinal flat mounts were used as negative controls, and very little TUNEL-staining was observed in the ONL under both treatment conditions.

### Western Blots

Whole retinas either from culture or freshly dissected were collected and added to 200 μL lysis buffer (20 mM Tris-HCl pH 7.5, 137 mM NaCl, 2 mM EDTA, 1% Nonidet P-40) containing a protease inhibitor cocktail (Roche). Samples were sonicated briefly and left on ice for 30 minutes before centrifuging and obtaining the supernatant. Protein quantities were assessed using the Bradford assay (BioRad), and 40 μg of total protein was mixed with Laemmli buffer, boiled for 5 minutes and loaded onto 10% gels. After transferring to PVDF membranes, a blocking step of 1 hour in 2% BSA was done, followed by an overnight incubation at 4° C. with the primary antibodies. Membranes were washed repeatedly then incubated for 1 hour with secondary antibody, washed again and developed using Western Lightning Plus ECL (PerkinElmer). Blots were scanned and quantified using ImageJ software. All primary antibodies were used at a 1:2000 dilution, with secondary 1:10000. To detect total protein for loading controls, membranes were stripped with an in-house buffer (62.5 mM. Tris-HCl pH 6.8, 2% SDS, and 0.7% 2-Mercaptoethanol) at 55° C. for 15 minutes, followed by extensive washing, blocking, and re-incubation with the primaries as just described.

### Rhodopsin Mutation Causes Progressive Thinning of the ONL

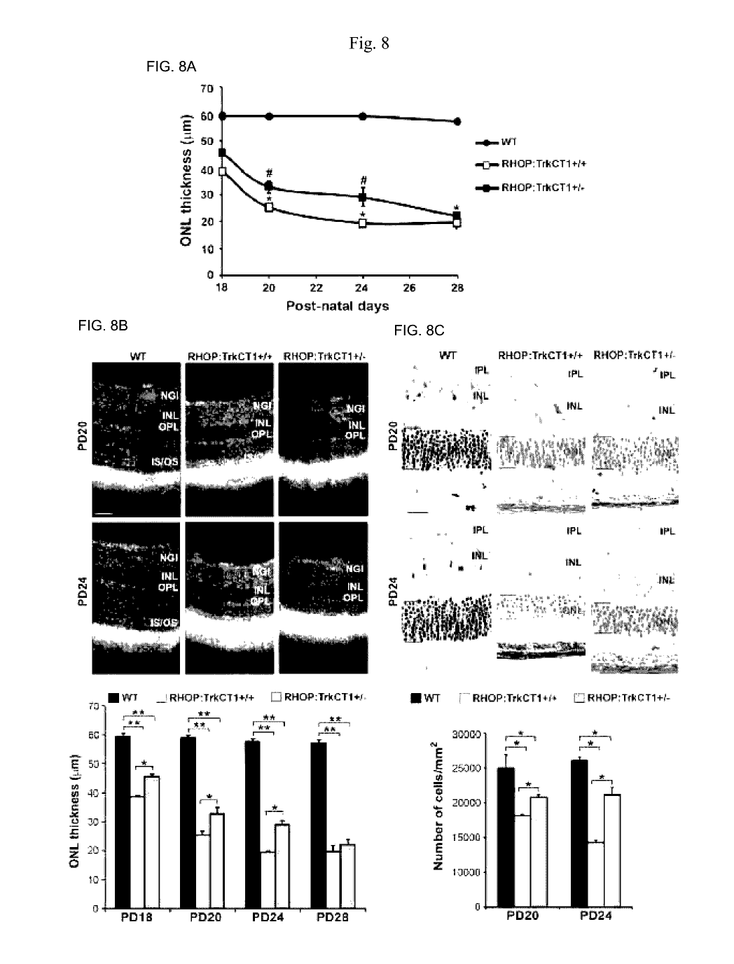

RHOP347S mouse model of RP displays a progressive retinal degeneration in which the retina loses nearly all photoreceptor cells. In some instances, these structural changes are examined using FD-OCT by measuring the thickness of the ONL. The thinning of the ONL is detectable as early as 18 days after birth, and rapidly progresses over time. The thickness of the ONL decreases to 50% (p<0.001) at post-natal day 24, and to 60% (p<0.001) at post-natal day 28, compared to the wild-type (FIG. 8a). Post-natal day 28 was established as the experimental endpoint since the massive thinning of the ONL is no longer measurable unbiasedly by FD-OCT beyond this time point.

### TrkC.T1 Heterozygosis (TrkC.T1+/−) Delays Thinning of the ONL in RHOP Mice

In some instances, it was demonstrated that deletion of TrkC.T1 reduces RGC death in glaucomatous eyes. Thus, it was to be determined whether TrkC.T1 has an impact on the degeneration of the ONL in RHOP mice. The thickness of the ONL was analyzed in wild-type, RHOP:TrkC.T1^{+/+} and RHOP:TrkC.T1^{+/-} mice. Although TrkC.T1^{-/-} mice does not have any abnormality and are viable, the complete deletion of TrkC.T1 (TrkC.T1^{-/-}) in a RHOP transgenic mice yields to small size-litters, and small size-offspring showing abnormal small eyes with close lids. Therefore, RHOP:TrkC.T1^{-/-} mice were excluded from this study. FD-OCT measurements showed that the thinning of the ONL layer in RHOP:TrkC.T1^{+/+} mice was less noticeable in RHOP:TrkC.T1^{+/-} mice spanning from post-natal day 18 to post-natal day 24 (FIGS. 8b and 8c). Concomitantly, it was observed that the number of photoreceptors was larger in RHOP:TrkC.T1^{+/-} mice compared to the RHOP:TrkC.T1^{+/+} offspring (p<0.001) at post-natal days 20 and 24 (FIG. 8c). At post-natal day 28, no protective effect on the ONL in RHOP:TrkC.T1^{+/-} mice was detected, and both, RHOP:TrkC.T1^{+/+} and RHOP:TrkC.T1^{+/-} samples showed similar values in the thickness measurements of the ONL (FIG. 8b). Thus, these data indicate that the deletion of TrkC.T1 has a temporary protective role in RP, and that genetic ablation of TrkC.T1 delays the degeneration of the ONL in RHOP:TrkC.T1^{+/+} mice. Interestingly enough, the elimination of just one copy of TrkC.T1 suffices to exert its protective effect.

### TrkC.T1 is Widely Up-Regulated in the Retina of RHOP Mice

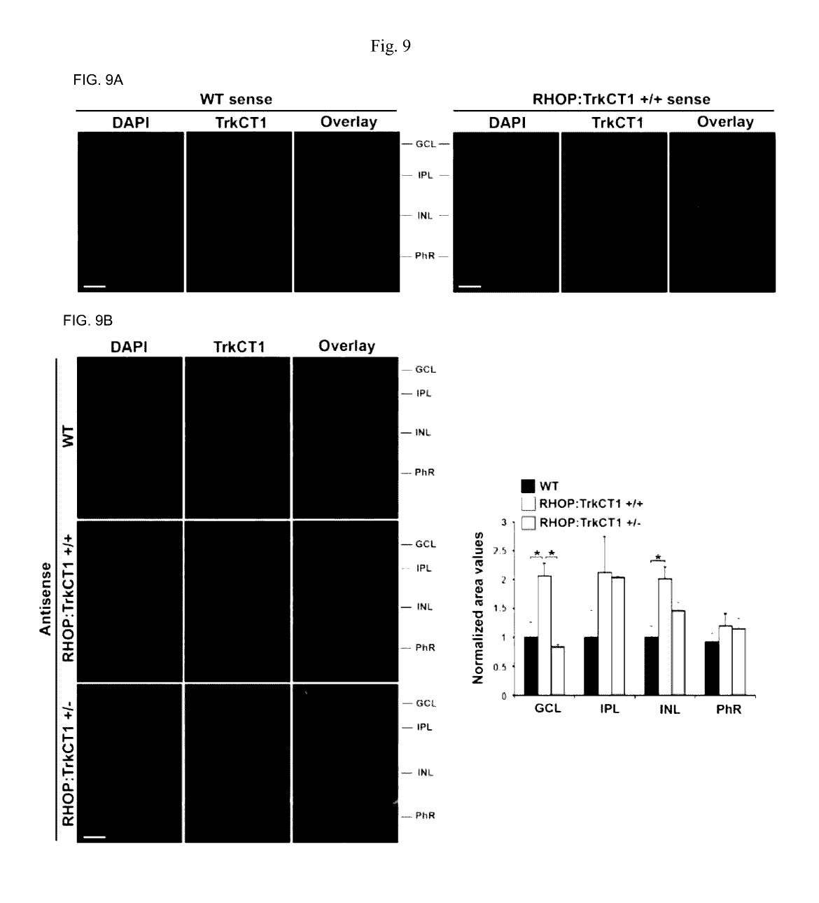

Next, to investigate whether TrkC.T1 is up-regulated in RHOP mice, retinal sections from wild-type and RHOP mice were studied by in situ mRNA hybridization using sense and antisense probes for TrkC.T1, and by immunofluorescence. In wild-type mice, TrkC.T1 mRNA was detected in the GCL and INL. At post-natal day 24, there was an increase of TrkC.T1 mRNA (p≦0.01) in the GCL and INL, of RHOP: TrkC.T1^{+/+} mice (FIG. 9b). Parallel studies with sections from RHOP: TrkC.T1^{-/-} animals showed that TrkC.T1 mRNA levels were reduced by 50% in the GCL (p<0.01). The mRNA levels were decreased by □ 25% in the INL, although this reduction did not reach significant values. No substantial difference in the levels of TrkC.T1 mRNA in the IPL and the PhR layer among the 3 animal genotypes was detected. The levels of TrkC.T1 mRNA in wild-type and RHOP: TrkC.T1^{+/+} sections tested for the control sense mRNA TrkC.T1 were under the limit of detection, as expected (FIG. 9a).

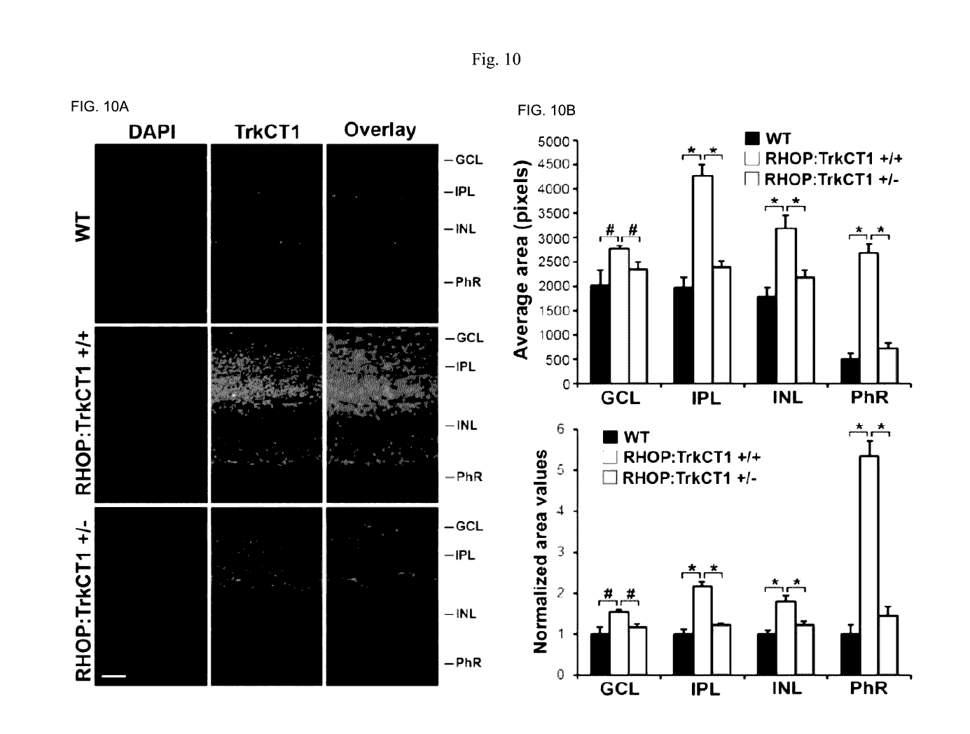

Immunohistochemistry data showed that there was a robust increase of TrkC.T1 protein in the IPL, and moderate in the GCL, INL and PhR layer in RHOP: TrkC.T1^{+/+} mice compared with the wild-type. Interestingly, TrkC.T1 staining was detected surrounding the cell's somata in the GCL, and intermingled with the cell bodies of the PhRs in the PhR layer. In RHOP: TrkC.T1^{-/-} mice, TrkC.T1 immunoreactivity was partially reduced in the GCL, IPL, INL and PhR layer (p<0.01) (FIG. 10).

Together, these results demonstrate that TrkC.T1 is up-regulated during RP and that this upregulation takes place mainly in the INL (where the cell bodies of Müller cells reside) and in the IPL and PhR layers (where projections of Müller cells are located). Note that the TrkC.T1 immunohistochemistry results were consistent with the in situ hybridization data, with the exception that the TrkC.T1 protein was strongly expressed in the IPL and PhR (FIG. 10) whereas TrkC.T1 mRNA levels were low. This observation suggests that TrkC.T1 protein are transported from the cell's soma to their projections in the inner and the outer nuclear layers in RHOP mice.

TrkC.T1 Regulates the Expression of p-Erk and p-Akt in RHOP Mice

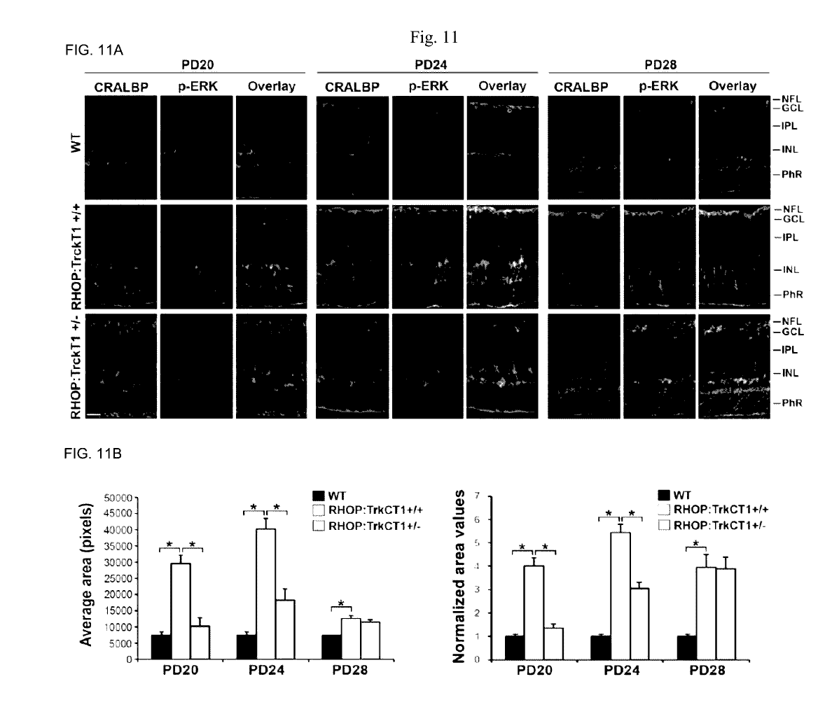

In some instances, TrkC.T1 downstream signaling induces Rac1 activation. Nonetheless, the effectors downstream TrkC.T1 remains elusive. Since p-Erk and p-Akt are targets of Rac1 in the nervous system it was to be investigated whether TrkC.T1 regulates the expression of p-Erk and p-Akt during RP. In healthy wild-type mice, a very weak activation of p-Erk was detected in the INL and GCL. At post-natal day 20, there was an increase of p-Erk in the soma of cells located in the INL, and in some projections spanning throughout the nerve fiber layer (NFL), and PhR layer in RHOP: TrkC.T1^{+/+} mice retinas. The p-Erk pattern of staining clearly co-localized with CRALBP and glutamine synthase (GS), indicating that p-Erk was exclusively expressed in Müller glial cells (FIG. 11, left panel and FIG. 12). At post-natal day 24, the levels of p-Erk were markedly increased in the Müller cells bodies, in their fibers projected towards the PhRs and in their processes ending in basal end-feet (FIG. 11, central panel and FIG. 12). At post-natal day 28, the p-Erk immunoreactivity was even more robust, and more Müller glial cells were labeled (FIG. 11, right panel). The sections of RHOP:TrkC.T1^{+/-} mice retinas showed a reduction of p-Erk signal in the somata and projections of Müller cells at post-natal days 20 and 24 (p<0.01) compared with the RHOP:TrkC.T1^{+/+} mice retinas (FIG. 11, left and central panels). At post-natal day 28, there was no difference in the levels of p-Erk between the RHOP:TrkC.T1^{+/+} and RHOP:TrkC.T1^{+/-} (FIG. 11, right panel).

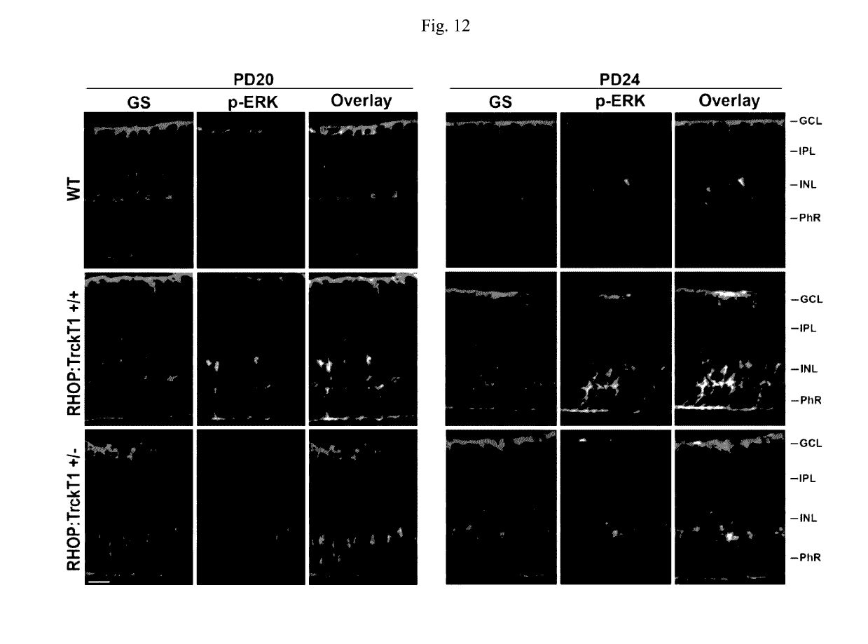

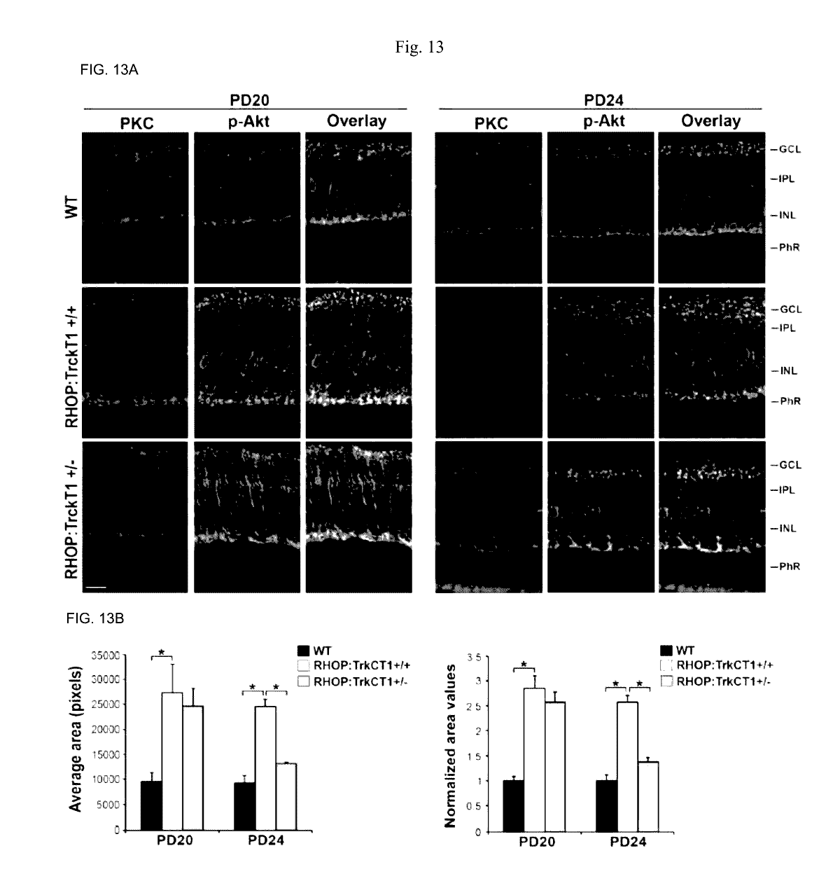

Similarly, the levels of p-Akt were increased in the RHOP:TrkC.T1^{+/+} animals compared to their wild-type littermates, at post-natal days 20 and 24 (FIG. 13). The p-Akt immunoreactivity was detected in the projections and somata of bipolar cells, as revealed by the co-localization between p-Akt and PKC, a specific marker of bipolar cells. In RHOP:TrkC.T1^{+/-} mice, the levels of p-Akt were reduced (p<0.01) only at post-natal day 24 (FIG. 13a, right panel and FIG. 13b). These data demonstrate that p-Erk is up-regulated specifically in Müller cells and p-Akt in bipolar cells during RP. Both, p-Erk and p-Akt levels were temporally decreased in RHOP:TrkC.T1^{+/-} mice, although p-Erk inhibition was more striking and sustained than p-Akt inhibition.

### Pharmacological Inhibition of TrkC Delays Retinal Degeneration and Inhibits MAPK/ERK and PI3K/pAkt Pathways

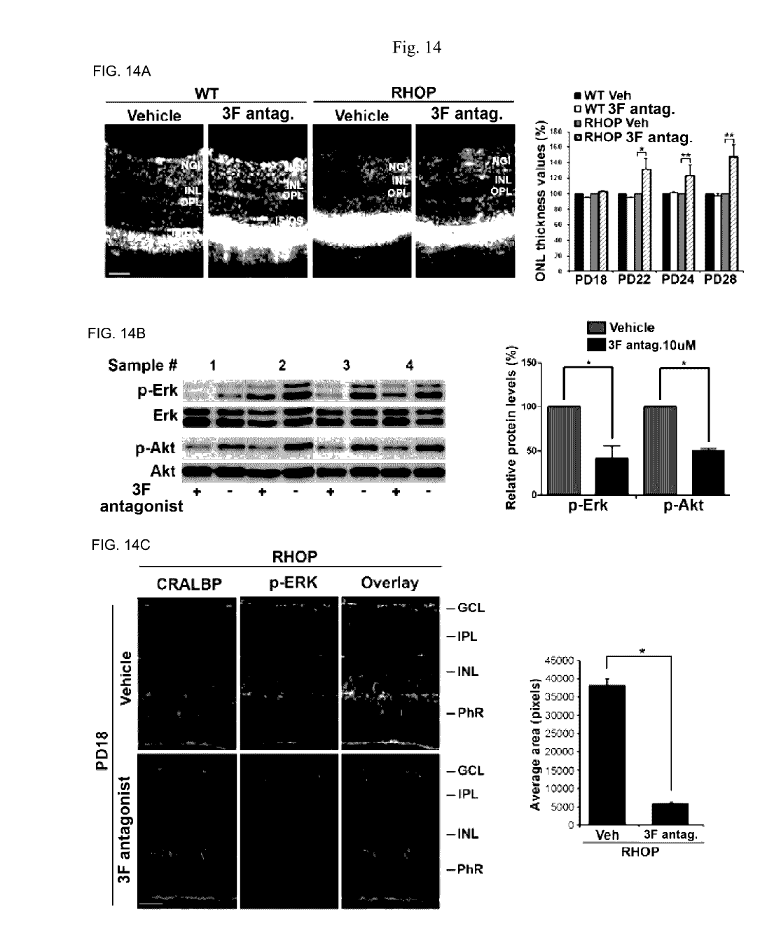

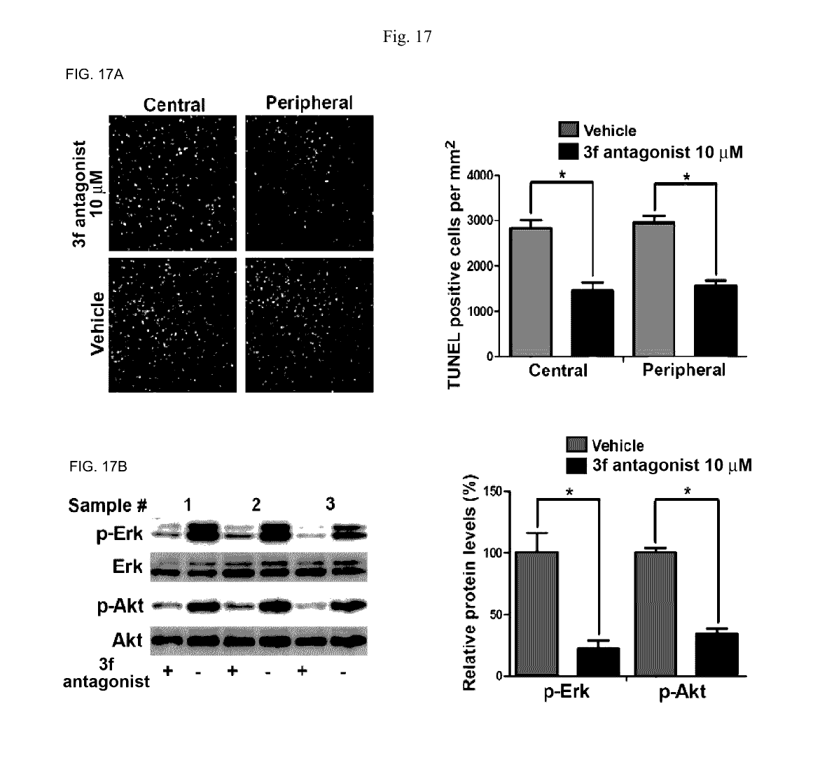

Since TrkC.T1 is increased in RHOP mice, it was to be investigated whether pharmacological inhibition of TrkC with a selective antagonist had protective role in the diseased retinas. The effect of compound 3F, a small molecule peptidomimetic (FIG. 2), was tested. The right eyes of the mice were injected with the TrkC antagonist and the left eyes with the vehicle, at post-natal day 17. The effect of intravitreal injections of 3F on the retina's morphology was examined by FD-OCT in wild-type and RHOP mice in both, vehicle (control) and 3F-injected eyes at post-natal days 18, 22, 24 and 28. The analysis of the FD-OCT images showed that the ONL thickness of 3F-injected eyes was bigger than the vehicle-injected eyes in RHOP retinas, at post-natal days 22, 24 and 28 (FIG. 14a). TUNEL assays developed on RHOP retinal explants showed less photoreceptor cell death on RHOP retinas injected with 3F (FIG. 17a), confirming the protective role of the TrkC antagonist on photoreceptors.

Given that p-Erk and p-Akt were increased during RP and the levels of p-Erk and p-Akt were decreased by genetic ablation of TrkC.T1 as showed in RHOP:TrkC.T1^{+/-} retinas, the effect of pharmacological inhibition of TrkC on the expression of p-Erk and p-Akt protein in RHOP mice retinas was examined by western blot (FIG. 14b) and immunohistochemistry (FIG. 14c), at post-natal day 18 (24 h after intravitreal injections). Densitometric analysis of western blots revealed a reduction of p-Erk (□40%) and p-Akt (□50%) in RHOP mice retinas treated with 3F compared to the vehicle-injected eyes (FIG. 14b) Similar data were obtained in vitro using RHOP retinal explants (FIG. 17b). Likewise, the immunohistochemistry data showed a reduction of p-Erk in Müller cells and a decrease of p-Akt in bipolar cells in 3F-injected eyes compared to the vehicle, 24 h after the injections (FIG. 14c and FIG. 12).

All together, these results indicate that pharmacological inhibition of TrkC has a protective role on the retinal degeneration in the RP model. The deleterious effect of TrkC.T1 seems to be mediated by activation of MAPK/ERK signaling pathways.

### TrkC.T1 Directly Activates the MAPK/ERK Signaling Pathway in Müller Glial Cells

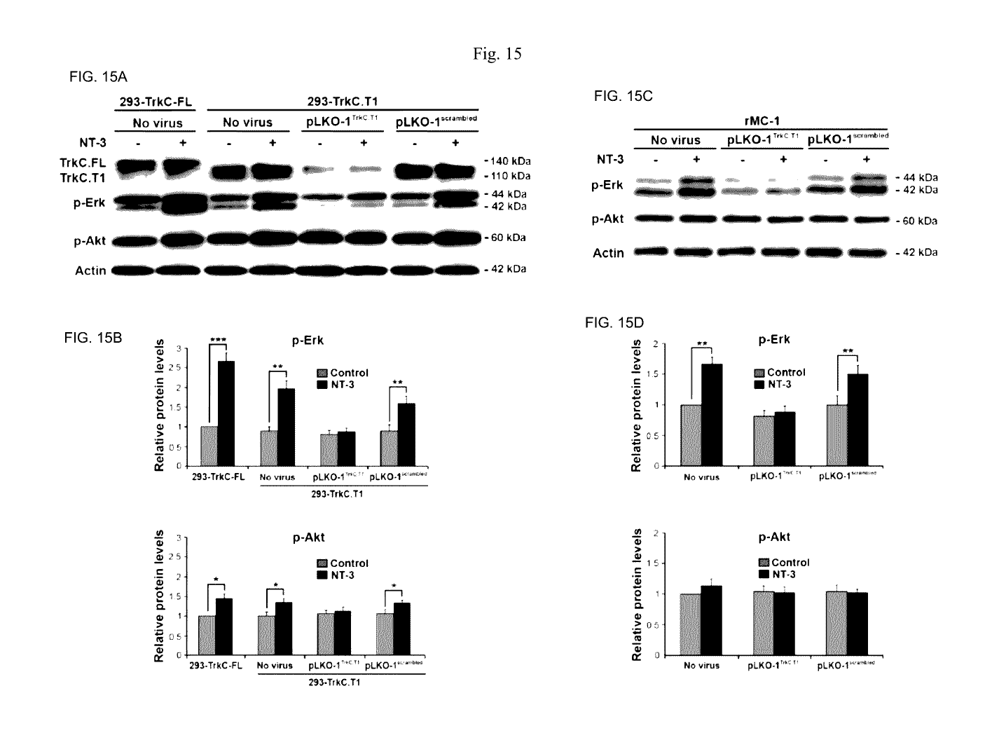

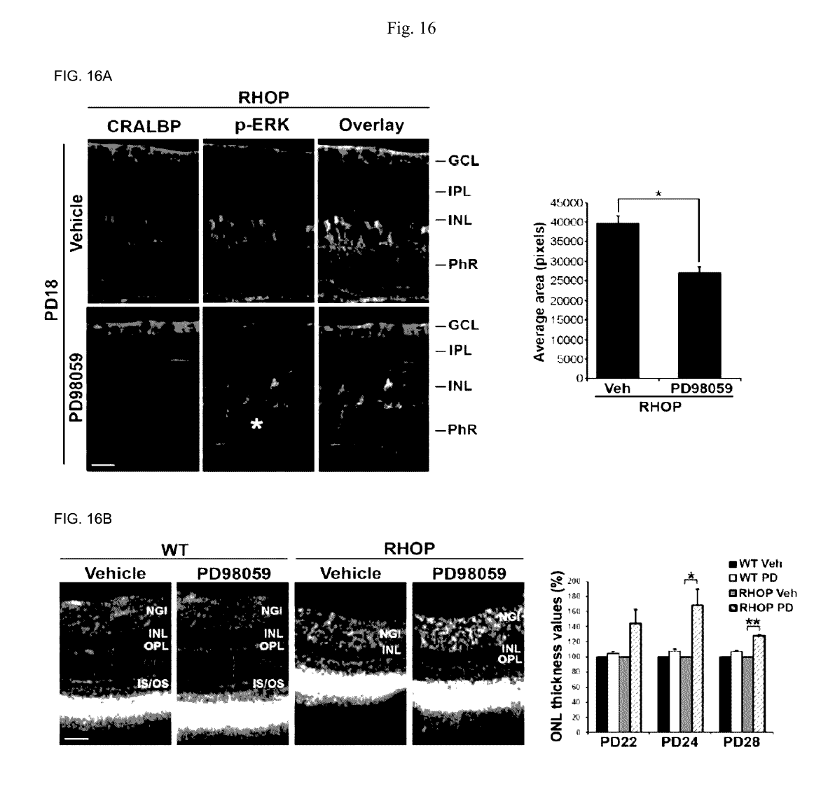

Since TrkC.T1 is mainly expressed in the retinal layers where Müller glial cells reside, and p-Erk is up-regulated in Müller glial cells during RP, it needed to be investigated whether TrkC.T1 directly activates downstream p-Erk in the mentioned cell type. To this end, the potency of the lentivirus PLKO.1^{TrkC.T1} (expressing a unique shRNA sequence that destabilizes TrkC.T1 mRNA) to silence TrkC.T1 mRNA in the HEK293 cell expression system was characterized. 293-TrkC-FL and non-infected 293-TrkC.T1 or 293-TrkC.T1 infected cells with lentiviral pLKO 1^{scrambled} or pLKO-1^{TrkC.T1} were treated with NT-3 4 nM for 10 min. Infection with PLKO.1^{TrkC.T1} almost completely abolished TrkC.T1 protein and reduced p-Erk expression in 293-TrkC.T1 infected cells with lentiviral pLKO-1^{TrkC.T1} compared to lentivirus PLKO.1^{scrambled}-infected 293-TrkC.T1 cells, or control non-infected cells (FIGS. 15a and 15b). NT-3 stimulation induced a □ 2.5-fold increase in p-Erk (p<0.001) and □ 1.5 in p-Akt levels (p<0.05) in 293-TrkC-FL cells. Non-infected 293-TrkC.T1 cells or pLKO-1^{scrambled} infected 293-TrkC.T1 cells showed a □ 2-fold and □ 1.5 increase in p-Erk levels (p<0.01), respectively, and a □1.5-fold increase in p-Akt (p<0.05), after NT-3 stimulation. No changes in p-Akt were detected in 293-TrkC.T1 infected cells with lentiviral pLKO-^{1TrkC.T1} (FIGS. 15a and 15b). These data indicate that infection with PLKO.1^{TrkC.T1} is able to knockdown TrkC.T1 and prevent p-Erk activation after NT-3 stimulation in 293-TrkC.T1 cells.

Next, to further corroborate the findings in a de facto Müller glial cell system, endogenous TrkC.T1 mRNA in r-MC-1 cells was silenced, using the same experimental approach as for HEK293 cells. r-MC-1 cells were infected with either PLKO.1^{TrkC.T1} or PLKO.1^{scrambled}, and stimulated with 4 nM NT-3 for 10 min. NT-3 stimulation induced a □ 1.5-fold increase in p-Erk protein levels in non-infected cells and rMC-1 cells infected with control lentivirus PLKO.1^{scrambled}. However, no increase in p-Erk levels in rMC-1 cells infected with PLKO.1^{TrkC T}1 was observed. The levels of p Akt did not change in neither rMC-1 infected nor rMC-1 wild-type cells (FIGS. 15c and 15d). This finding agrees with the results in vivo where p-Akt expression was exclusively detected in bipolar cells rather than in Müller glial cells (FIG. 13).

The data show that TrkC.T1 is expressed in Müller glial cells and that selective silencing of TrkC.T1 abolished NT-3-mediated p-Erk activation, but not p-Akt. In some instances, these results suggest that downstream activation of the MAPK/ERK signaling cascade by TrkC.T1 in Müller glial cell mediate retina degeneration during RP. Furthermore, these data point to a paracrine or non-cell autologous mechanism of PhR cell death.

### Inhibition of MAPK/ERK Activity Delays Retinal Degeneration

To further verify the involvement of p-Erk activation in retina degeneration, the direct interference with the MAPK/ERK signaling cascade was further examined to see if it also had a protective effect on the ONL degeneration during RP. Intravitreal injections of PD90859 or vehicle (DMSO) were done into the right (treated) or left (control) eye respectively, at post-natal day 17. First, the effect of PD98059 on p-Erk was examined by immunohistochemistry 24 h after intravitreal injections (at post-natal day 18). The MAPK/ERK inhibitor did block p-Erk (030%, p<0.01) in Müller cells compared to the vehicle-injected eye. The p-Erk signal was abolished in the somata and projections of Müller cells in the inner and outer (asterisk) retinal layers (FIG. 15a).

Next, the effect of PD98059 on the structural changes that the ONL undergoes during RP was examined by FD-OCT at post-natal days 22, 24 and 28 (FIG. 15b). In both, wild-type and RHOP mice, each vehicle injected-eye was arbitrarily established as 100% of the ONL thickness and used as internal control in our experiments. The results demonstrate that the ONL thickness was slightly bigger in the PD98050-injected eyes compared to the vehicle at post-natal day 22, although did not reach significant values. At post-natal day 24, the MAPK/ERK inhibitor had a clear protective effect on the ONL degeneration in comparison with the contralateral control eye (p<0.01). At post-natal day 28, the thickness of the ONL is still larger in the PD98050-injected eyes compared to the vehicle (p<0.001). However, the protection yielded by the MAPK/ERK inhibitor is not as robust as at post-natal day 24.

Together, these data demonstrate that direct inhibition of the MAPK/ERK signaling pathway temporally delays the degeneration of the ONL. Taken all together, these findings indicate that p-Erk activation might trigger PhR cell death in the RHOP mice model of RP.

### Example 3

Table 1 illustrates exemplary sequences disclosed herein. The DNA sequences disclosed in Table 1 encode TrkC shRNAs described herein.

TABLE 1

| SEQ ID | |
|---|---|
| NOs | |
| 1 | GGACAATAGAGATCATCTAGT |
| 2 | CCGGGGACAATAGAGATCATCTAGTCTCGAGACTA |
| | GATGATCTCTATTGTCCTTTTTG |
| 3 | AATTCAAAAAGGACAATAGAGATCATCTAGTCTCG |
| | AGACTAGATGATCTCTATTGTCC |
| 4 | CCGGGGACATTCCAAGCCTCTTAACCTCGAGGTTA |
| | AGAGGCTTGGAATGTCCTTTTTG |
| 5 | AATTCAAAAAGGACATTCCAAGCCTCTTAACCTCG |
| | AGGTTAAGAGGCTTGGAATGTCC |
| 6 | CCGGCATGGTTTCAGAGAAATTATGCTCGAGCATA |
| | ATTTCTCTGAAACCATGTTTTTG |
| 7 | AATTCAAAAACATGGTTTCAGAGAAATTATGCTCG |
| | AGCATAATTTCTCTGAAACCATG |
| 114 | GGUAUAAGUGCACACUGAAUA |
| 115 | CACUGAAUAGUCUAAUCUACA |
| 116 | GAGAGUCAAACAAUGUUAAGG |
| 117 | GAGAAGAGUUCUAUGGUUAUC |
| 118 | GACAAAGCAGUGUGCUCUAAU |
| 119 | GGAUGUGUUUGUACUUGCAGA |
| 8 | CCTAAGGTTAAGTCGCCCTCG |
| (control) | |

Table 2 illustrates sequences of the full-length and truncated isoforms of TrkC and TrkB.

TABLE 2

| | |
|---|---|
| TrkC Full-length | MDVSLCPAKCSFWRIFLLGSVWLDYVG |
| GenBank: | SVLACPANCVCSKTEINCRRPDDGNLF |
| CAA12029.1 | PLLEGQDSGNSNGNASINITDISRNIT |
| SEQ ID NO: 9 | SIHIENWRSLHTLNAVDMELYTGLQKL |
| | TIKNSGLRSIQPRAFAKNPHLRYINLS |
| | SNRLTTLSWQLFQTLSLRELQLEQNFF |
| | NCSCDIRWMQLWQEQGEAKLNSQNLYC |
| | INADGSQLPLFRMNISQCDLPEISVSH |
| | VNLTVREGDNAVITCNGSGSPLPDVDW |
| | IVTGLQSINTHQTNLNWTNVHAINLTL |
| | VNVTSEDNGFTLTCIAENVVGMSNASV |
| | ALTVYYPPRVVSLEEPELRLEHCIEFV |
| | VRGNPPPTLHWLHNGQPLRESKIIHVE |
| | YYQEGEISEGCLLFNKPTHYNNGNYTL |
| | IAKNPLGTANQTINGHFLKEPFPESTD |
| | NFILFDEVSPTPPITVTHKPEEDTFGV |
| | SIAVGLAAFACVLLVVLFVMINKYGRR |
| | SKFGMKGPVAVISGEEDSASPLHHINH |
| | GITTPSSLDAGPDTVVIGMTRIPVIEN |
| | PQYFRQGHNCHKPDTYVQHIKRRDIVL |
| | KRELGEGAFGKVFLAECYNLSPTKDKM |
| | LVAVKALKDPTLAARKDFQREAELLTN |
| | LQHEHIVKFYGVCGDGDPLIMVFEYMK |
| | HGDLNKFLRAHGPDAMILVDGQPRQAK |
| | GELGLSQMLHIASQIASGMVYLASQHF |
| | VHRDLATRNCLVGANLLVKIGDFGMSR |
| | DVYSTDYYRLFNPSGNDFCIWCEVGGH |
| | TMLPIRWMPPESIMYRKFTTESDVWSF |
| | GVILWEIFTYGKQPWFQLSNTEVIECI |
| | TQGRVLERPRVCPKEVYDVMLGCWQRE |
| | PQQRLNIKEIYKILHALGKATPIYLDI |
| | LG |
| TrkC Full-length | MDVSLCPAKCSFWRIFLLGSVWLDYVG |
| UniProtKB Accession | SVLACPANCVCSKTEINCRPDDGNLFP |
| Number: Q16288-3 | LLEGQDSGNSNGNASINITDISRNITS |
| SEQ ID NO: 110 | IHIENWRSLHTLNAVDMELYTGLQKLT |
| | IKNSGLRSIQPRAFAKNPHLRYINLSS |
| | NRLTTLSWQLFQTLSLRELQLEQNFFN |
| | CSCDIRWMQLWQEQGEAKLNSQNLYCI |
| | NADGSQLPLFRMNISQCDLPEISVSHV |
| | NLTVREGDNAVITCNGSGSPLPDVDWI |
| | VTGLQSINTHQTNLNWTNVHAINLTLV |
| | NVTSEDNGFTLTCIAENVVGMSNASVA |
| | LTVYYPPRVVSLEEPELRLEHCIEFVV |
| | RGNPPPTLHWLHNGQPLRESKIIHVEY |
| | YQEGEISEGCLLFNKPTHYNNGNYTLI |
| | AKNPLGTANQTINGHFLKEPFPESTDN |
| | FILFDEVSPTPPITVTHKPEEDTFGVS |
| | IAVGLAAFACVLLVVLFVMINKYGRRS |
| | KFGMKGPVAVISGEEDSASPLHHINHG |
| | ITTPSSLDAGPDTVVIGMTRIPVIENP |
| | QYFRQGHNCHKPDTYVQHIKRRDIVLK |
| | RELGEGAFGKVFLAECYNLSPTKDKML |
| | VAVKALKDPTLAARKDFQREAELLTNL |
| | QHEHIVKFYGVCGDGDPLIMVFEYMKH |
| | GDLNKFLRAHGPDAMILVDGQPRQAKG |
| | ELGLSQMLHIASQIASGMVYLASQHFV |
| | HRDLATRNCLVGANLLVKIGDFGMSRD |
| | VYSTDYYRVGGHTMLPIRWMPPESIMY |
| | RKFTTESDVWSFGVILWEIFTYGKQPW |
| | FQLSNTEVIECITQGRVLERPRVCPKE |
| | VYDVMLGCWQREPQQRLNIKEIYKILH |
| | ALGKATPIYLDILG |
| TrkC Full-length | MDVSLCPAKCSFWRIFLLGSVWLDYVG |
| UniProtKB Accession | SVLACPANCVCSKTEINCRPDDGNLFP |
| Number: Q16288-4 | LLEGQDSGNSNGNASINITDISRNITS |
| SEQ ID NO: 111 | IHIENWRSLHTLNAVDMELYTGLQKLT |
| | IKNSGLRSIQPRAFAKNPHLRYINLSS |
| | NRLTTLSWQLFQTLSLRELQLEQNFFN |
| | CSCDIRWMQLWQEQGEAKLNSQNLYCI |
| | NADGSQLPLFRMNISQCDLPEISVSHV |
| | NLTVREGDNAVITCNGSGSPLPDVDWI |
| | VTGLQSINTHQTNLNWTNVHAINLTLV |
| | NVTSEDNGFTLTCIAENVVGMSNASVA |
| | LTVYYPPRVVSLEEPELRLEHCIEFVV |
| | RGNPPPTLHWLHNGQPLRESKIIHVEY |
| | YQEGEISEGCLLFNKPTHYNNGNYTLI |
| | AKNPLGTANQTINGHFLKEPFPVDEVS |
| | PTPPITVTHKPEEDTFGVSIAVGLAAF |
| | ACVLLVVLFVMINKYGRRSKFGMKGPV |
| | AVISGEEDSASPLHHINHGITTPSSLD |
| | AGPDTVVIGMTRIPVIENPQYFRQGHN |
| | CHKPDTYVQHIKRRDIVLKRELGEGAF |
| | GKVFLAECYNLSPTKDKMLVAVKALKD |
| | PTLAARKDFQREAELLTNLQHEHIVKF |
| | YGVCGDGDPLIMVFEYMKHGDLNKFLR |
| | AHGPDAMILVDGQPRQAKGELGLSQML |
| | HIASQIASGMVYLASQHFVHRDLATRN |
| | CLVGANLLVKIGDFGMSRDVYSTDYYR |
| | LFNPSGNDFCIWCEVGGHTMLPIRWMP |
| | PESIMYRKFTTESDVWSFGVILWEIFT |
| | YGKQPWFQLSNTEVIECITQGRVLERP |
| | RVCPKEVYDVMLGCWQREPQQRLNIKE |
| | IYKILHALGKATPIYLDILG |
| TrkC Full-length | MDVSLCPAKCSFWRIFLLGSVWLDYVG |
| UniProtKB Accession | SVLACPANCVCSKTEINCRPDDGNLFP |
| Number: Q16288-5 | LLEGQDSGNSNGNASINITDISRNITS |
| SEQ ID NO: 112 | IHIENWRSLHTLNAVDMELYTGLQKLT |
| | IKNSGLRSIQPRAFAKNPHLRYINLSS |
| | NRLTTLSWQLFQTLSLRELQLEQNFFN |
| | CSCDIRWMQLWQEQGEAKLNSQNLYCI |
| | NADGSQLPLFRMNISQCDLPEISVSHV |
| | NLTVREGDNAVITCNGSGSPLPDVDWI |
| | VTGLQSINTHQTNLNWTNVHAINLTLV |
| | NVTSEDNGFTLTCIAENVVGMSNASVA |
| | LTVYYPPRVVSLEEPELRLEHCIEFVV |
| | RGNPPPTLHWLHNGQPLRESKIIHVEY |
| | YQEGEISEGCLLFNKPTHYNNGNYTLI |
| | AKNPLGTANQTINGHFLKEPFPVDEVS |
| | PTPPITVTHKPEEDTFGVSIAVGLAAF |
| | ACVLLVVLFVMINKYGRRSKFGMKGPV |
| | AVISGEEDSASPLHHINHGITTPSSLD |
| | AGPDTVVIGMTRIPVIENPQYFRQGHN |
| | CHKPDTYVQHIKRRDIVLKRELGEGAF |
| | GKVFLAECYNLSPTKDKMLVAVKALKD |
| | PTLAARKDFQREAELLTNLQHEHIVKF |
| | YGVCGDGDPLIMVFEYMKHGDLNKFLR |
| | AHGPDAMILVDGQPRQAKGELGLSQML |
| | HIASQIASGMVYLASQHFVHRDLATRN |
| | CLVGANLLVKIGDFGMSRDVYSTDYYR |
| | VGGHTMLPIRWMPPESIMYRKFTTESD |
| | VWSFGVILWEIFTYGKQPWFQLSNTEV |
| | IECITQGRVLERPRVCPKEVYDVMLGC |
| | WQREPQQRLNIKEIYKILHALGKATPI |
| | YLDILG |
| TrkC.T1 | MDVSLCPAKCSFWRIFLLGSVWLDYVG |
| GenBank: | SVLACPANCVCSKTEINCRRPDDGNLF |
| AAB33112.1 | PLLEGQDSGNSNGNANINITDISRNIT |
| SEQ ID NO: 10 | SIHIENWRSLHTLNAVDMELYTGLQKL |
| | TIKNSGLRSIQPRAFAKNPHLRYINLS |
| | SNRLTTLSWQLFQTLSLRELQLEQNFF |
| | NCSCDIRWMQLWQEQGEAKLNSQNLYC |
| | INADGSQLPLFRMNISQCDLPEISVSH |
| | VNLTVREGDNAVITCNGSGSPLPDVDW |
| | IVTGLQSINTHQTNLNWTNVHAINLTL |
| | VNVTSEDNGFTLTCIAENVVGMSNASV |
| | ALTVYYPPRVVSLEEPELRLEHCIEFV |
| | VRGNPPPTLHWLHNGQPLRESKIIHVE |
| | YYQEGEISEGCLLFNKPTHYNNGNYTL |
| | IAKNPLGTANQTINGHFLKEPFPESTD |
| | NFILFDEVSPTPPITVTHKPEEDTFGV |
| | SIAVGLAAFACVLLVVLFVMINKYGRR |
| | SKFGMKGPVAVISGEEDSASPLHHINH |
| | GITTPSSLDAGPDTVVIGMTRIPVIEN |
| | PQYFRQGHNCHKPDTWVFSNIDNHGIL |
| | NLKDNRDHLVPSTHYIYEEPEVQSGEV |
| | SYPRSHGFREIMLNPISLPGHSKPLNH |
| | GIYVEDVNVYFSKGRHGF |
| TrkC.T1 | MDVSLCPAKCSFWRIFLLGSVWLDYVG |
| UniProtKB Accession | SVLACPANCVCSKTEINCRPDDGNLFP |
| Number: Q16288-2 | LLEGQDSGNSNGNASINITDISRNITS |
| SEQ ID NO: 113 | IHIENWRSLHTLNAVDMELYTGLQKLT |
| | IKNSGLRSIQPRAFAKNPHLRYINLSS |
| | NRLTTLSWQLFQTLSLRELQLEQNFFN |
| | CSCDIRWMQLWQEQGEAKLNSQNLYCI |
| | NADGSQLPLFRMNISQCDLPEISVSHV |
| | NLTVREGDNAVITCNGSGSPLPDVDWI |
| | VTGLQSINTHQTNLNWTNVHAINLTLV |
| | NVTSEDNGFTLTCIAENVVGMSNASVA |
| | LTVYYPPRVVSLEEPELRLEHCIEFVV |
| | RGNPPPTLHWLHNGQPLRESKIIHVEY |
| | YQEGEISEGCLLFNKPTHYNNGNYTLI |
| | AKNPLGTANQTINGHFLKEPFPESTDN |
| | FILFDEVSPTPPITVTHKPEEDTFGVS |
| | IAVGLAAFACVLLVVLFVMINKYGRRS |
| | KFGMKGPVAVISGEEDSASPLHHINHG |
| | ITTPSSLDAGPDTVVIGMTRIPVIENP |
| | QYFRQGHNCHKPDTWVFSNIDNHGILN |
| | LKDNRDHLVPSTHYIYEEPEVQSGEVS |
| | YPRSHGFREIMLNPISLPGHSKPLNHG |
| | IYVEDVNVYFSKGRHGF |
| TrkB isoform c | MSSWIRWHGPAMARLWGFCWLVVGFWR |
| GenBank: | AAFACPTSCKCSASRIWCSDPSPGIVA |
| AAB33109.1 | FPRLEPNSVDPENITEIFIANQKRLEI |
| SEQ ID NO: 11 | INEDDVEAYVGLRNLTIVDSGLKFVAH |
| | KAFLKNSNLQHINFTRNKLTSLSRKHF |
| | RHLDLSELILVGNPFTCSCDIMWIKTL |
| | QEAKSSPDTQDLYCLNESSKNIPLANL |
| | QIPNCGLPSANLAAPNLTVEEGKSITL |
| | SCSVAGDPVPNMYWDVGNLVSKHMNET |
| | SHTQGSLRITNISSDDSGKQISCVAEN |
| | LVGEDQDSVNLTVHFAPTITFLESPTS |
| | DHHWCIPFTVKGNPKPALQWFYNGAIL |
| | NESKYICTKIHVTNHTEYHGCLQLDNP |
| | THMNNGDYTLIAKNEYGKDEKQISAHF |
| | MGWPGIDDGANPNYPDVIYEDYGTAAN |
| | DIGDTTNRSNEIPSTDVTDKTGREHLS |
| | VYAVVVIASVVGFCLLVMLFLLKLARH |
| | SKFGMKGPASVISNDDDSASPLHHISN |
| | GSNTPSSSEGGPDAVIIGMTKIPVIEN |
| | PQYFGITNSQLKPDTFVQHIKRHNIVL |
| | KRELGEGAFGKVFLAECYNLCPEQDKI |
| | LVAVKTLKDASDNARKDFHREAELLTN |
| | LQHEHIVKFYGVCVEGDPLIMVFEYMK |
| | HGDLNKFLRAHGPDAVLMAEGNPPTEL |
| | TQSQMLHIAQQIAAGMVYLASQHFVHR |
| | DLATRNCLVGENLLVKIGDFGMSRDVY |
| | STDYYRVGGHTMLPIRWMPPESIMYRK |
| | FTTESDVWSLGVVLWEIFTYGKQPWYQ |
| | LSNNEVIECITQGRVLQRPRTCPQEVY |
| | ELMLGCWQREPHMRKNIKGIHTLLQNL |
| | AKASPVYLDILG |
| TrkB.T1 | MSSWIRWHGPAMARLWGFCWLVVGFWR |
| (TrkB isoform b) | AAFACPTSCKCSASRIWCSDPSPGIVA |
| GenBank: | FPRLEPNSVDPENITEIFIANQKRLEI |
| AAM77876.1 | INEDDVEAYVGLRNLTIVDSGLKFVAH |
| SEQ ID NO: 12 | KAFLKNSNLQHINFTRNKLTSLSRKHF |
| | RHLDLSELILVGNPFTCSCDIMWIKTL |
| | QEAKSSPDTQDLYCLNESSKNIPLANL |
| | QIPNCGLPSANLAAPNLTVEEGKSITL |
| | SCSVAGDPVPNMYWDVGNLVSKHMNET |
| | SHTQGSLRITNISSDDSGKQISCVAEN |
| | LVGEDQDSVNLTVHFAPTITFLESPTS |
| | DHHWCIPFTVKGNPKPALQWFYNGAIL |
| | NESKYICTKIHVTNHTEYHGCLQLDNP |
| | THMNNGDYTLIAKNEYGKDEKQISAHF |
| | MGWPGIDDGANPNYPDVIYEDYGTAAN |
| | DIGDTTNRSNEIPSTDVTDKTGREHLS |
| | VYAVVVIASVVGFCLLVMLFLLKLARH |
| | SKFGMKGFVLFHKIPLDG |
| TrkB isoform a | MSSWIRWHGPAMARLWGFCWLVVGFWR |
| Origene Accession | AAFACPTSCKCSASRIWCSDPSPGIVA |
| Number: | FPRLEPNSVDPENITEIFIANQKRLEI |
| NP_006171.2 | INEDDVEAYVGLRNLTIVDSGLKFVAH |
| SEQ ID NO: 120 | KAFLKNSNLQHINFTRNKLTSLSRKHF |
| | RHLDLSELILVGNPFTCSCDIMWIKTL |
| | QEAKSSPDTQDLYCLNESSKNIPLANL |
| | QIPNCGLPSANLAAPNLTVEEGKSITL |
| | SCSVAGDPVPNMYWDVGNLVSKHMNET |
| | SHTQGSLRITNISSDDSGKQISCVAEN |
| | LVGEDQDSVNLTVHFAPTITFLESPTS |
| | DHHWCIPFTVKGNPKPALQWFYNGAIL |
| | NESKYICTKIHVTNHTEYHGCLQLDNP |
| | THMNNGDYTLIAKNEYGKDEKQISAHF |
| | MGWPGIDDGANPNYPDVIYEDYGTAAN |
| | DIGDTTNRSNEIPSTDVTDKTGREHLS |
| | VYAVVVIASVVGFCLLVMLFLLKLARH |
| | SKFGMKDFSWFGFGKVKSRQGVGPASV |
| | ISNDDDSASPLHHISNGSNTPSSSEGG |
| | PDAVIIGMTKIPVIENPQYFGITNSQL |
| | KPDTFVQHIKRHNIVLKRELGEGAFGK |
| | VFLAECYNLCPEQDKILVAVKTLKDAS |
| | DNARKDFHREAELLTNLQHEHIVKFYG |
| | VCVEGDPLIMVFEYMKHGDLNKFLRAH |
| | GPDAVLMAEGNPPTELTQSQMLHIAQQ |
| | IAAGMVYLASQHFVHRDLATRNCLVGE |
| | NLLVKIGDFGMSRDVYSTDYYRVGGHT |
| | MLPIRWMPPESIMYRKFTTESDVWSLG |
| | VVLWEIFTYGKQPWYQLSNNEVIECIT |
| | QGRVLQRPRTCPQEVYELMLGCWQREP |
| | HMRKNIKGIHTLLQNLAKASPVYLDIL |
| | G |
| TrkB isoform d | MSSWIRWHGPAMARLWGFCWLVVGFWR |
| (TrkB-T-Shc) | AAFACPTSCKCSASRIWCSDPSPGIVA |
| Origene Accession | FPRLEPNSVDPENITEIFIANQKRLEI |
| Number: | INEDDVEAYVGLRNLTIVDSGLKFVAH |
| NP_001018075.1 | KAFLKNSNLQHINFTRNKLTSLSRKHF |
| SEQ ID NO: 121 | RHLDLSELILVGNPFTCSCDIMWIKTL |
| | QEAKSSPDTQDLYCLNESSKNIPLANL |
| | QIPNCGLPSANLAAPNLTVEEGKSITL |
| | SCSVAGDPVPNMYWDVGNLVSKHMNET |
| | SHTQGSLRITNISSDDSGKQISCVAEN |
| | LVGEDQDSVNLTVHFAPTITFLESPTS |
| | DHHWCIPFTVKGNPKPALQWFYNGAIL |
| | NESKYICTKIHVTNHTEYHGCLQLDNP |
| | THMNNGDYTLIAKNEYGKDEKQISAHF |
| | MGWPGIDDGANPNYPDVIYEDYGTAAN |
| | DIGDTTNRSNEIPSTDVTDKTGREHLS |
| | VYAVVVIASVVGFCLLVMLFLLKLARH |
| | SKFGMKDFSWFGFGKVKSRQGVGPASV |
| | ISNDDDSASPLHHISNGSNTPSSSEGG |
| | PDAVIIGMTKIPVIENPQYFGITNSQL |
| | KPDTWPRGSPKTA |
| TrkB isoform e | MSSWIRWHGPAMARLWGFCWLVVGFWR |
| (TrkB-T-Shc) | AAFACPTSCKCSASRIWCSDPSPGIVA |
| Origene Accession | FPRLEPNSVDPENITEIFIANQKRLEI |
| Number: | INEDDVEAYVGLRNLTIVDSGLKFVAH |
| NP_001018076.1 | KAFLKNSNLQHINFTRNKLTSLSRKHF |
| SEQ ID NO: 122 | RHLDLSELILVGNPFTCSCDIMWIKTL |
| | QEAKSSPDTQDLYCLNESSKNIPLANL |
| | QIPNCGLPSANLAAPNLTVEEGKSITL |
| | SCSVAGDPVPNMYWDVGNLVSKHMNET |
| | SHTQGSLRITNISSDDSGKQISCVAEN |
| | LVGEDQDSVNLTVHFAPTITFLESPTS |
| | DHHWCIPFTVKGNPKPALQWFYNGAIL |
| | NESKYICTKIHVTNHTEYHGCLQLDNP |
| | THMNNGDYTLIAKNEYGKDEKQISAHF |
| | MGWPGIDDGANPNYPDVIYEDYGTAAN |
| | DIGDTTNRSNEIPSTDVTDKTGREHLS |
| | VYAVVVIASVVGFCLLVMLFLLKLARH |
| | SKFGMKGPASVISNDDDSASPLHHISN |
| | GSNTPSSSEGGPDAVIIGMTKIPVIEN |
| | PQYFGITNSQLKPDTWPRGSPKTA |
| TrkB isoform f | MSSWIRWHGPAMARLWGFCWLVVGFWR |
| Origene Accession | AAFACPTSCKCSASRIWCSDPSPGIVA |
| Number: | FPRLEPNSVDPENITEIFIANQKRLEI |
| NP_001278866.1 | INEDDVEAYVGLRNLTIVDSGLKFVAH |
| SEQ ID NO: 123 | KAFLKNSNLQHINFTRNKLTSLSRKHF |
| | RHLDLSELILVGNPFTCSCDIMWIKTL |
| | QEAKSSPDTQDLYCLNESSKNIPLANL |
| | QIPNCGLPSANLAAPNLTVEEGKSITL |
| | SCSVAGDPVPNMYWDVGNLVSKHMNET |
| | SHTQGSLRITNISSDDSGKQISCVAEN |
| | LVGEDQDSVNLTVHYHHWCIPFTVKGN |
| | PKPALQWFYNGAILNESKYICTKIHVT |
| | NHTEYHGCLQLDNPTHMNNGDYTLIAK |
| | NEYGKDEKQISAHFMGWPGIDDGANPN |
| | YPDVIYEDYGTAANDIGDTTNRSNEIP |
| | STDVTDKTGREHLSVYAVVVIASVVGF |
| | CLLVMLFLLKLARHSKFGMKGFVLFHK |
| | IPLDG |

Table 3 illustrates miRNA sequences.

TABLE 3

| | miRNA | Sequence |
|---|---|---|
| SEQ ID NO: 13 | let-7b-3p | CUAUACAACCUAC |
| | | UGCCUUCCC |
| SEQ ID NO: 14 | let-7b-5p | UGAGGUAGUAGGU |
| | | UGUGUGGUU |
| SEQ ID NO: 15 | miR-1-3p | UGGAAUGUAAAGA |
| | | AGUAUGUAU |
| SEQ ID NO: 16 | miR-1-5p | ACAUACUUCUUUA |
| | | UAUGCCCAU |
| SEQ ID NO: 17 | miR-9-3p | AUAAAGCUAGAUA |
| | | ACCGAAAGU |
| SEQ ID NO: 18 | miR-9-5p | UCUUUGGUUAUCU |
| | | AGCUGUAUGA |
| SEQ ID NO: 19 | miR-10a-3p | CAAAUUCGUAUCU |
| | | AGGGGAAUA |
| SEQ ID NO: 20 | miR-10a-5p | UACCCUGUAGAUC |
| | | CGAAUUUGUG |
| SEQ ID NO: 21 | miR-15a-3p | CAGGCCAUAUUGU |
| | | GCUGCCUCA |
| SEQ ID NO: 22 | miR-15a-5p | UAGCAGCACAUAA |
| | | UGGUUUGUG |
| SEQ ID NO: 23 | miR-16-1-3p | CCAGUAUUAACUG |
| | | UGCUGCUGA |
| SEQ ID NO: 24 | miR-16-2-3p | CCAAUAUUACUGU |
| | | GCUGCUUUA |
| SEQ ID NO: 25 | miR-16-5p | UAGCAGCACGUAA |
| | | AUAUUGGCG |
| SEQ ID NO: 26 | miR-17-3p | ACUGCAGUGAAGG |
| | | CACUUGUAG |
| SEQ ID NO: 27 | miR-17-5p | CAAAGUGCUUACA |
| | | GUGCAGGUAG |
| SEQ ID NO: 28 | miR-18a-3p | ACUGCCCUAAGUG |
| | | CUCCUUCUGG |
| SEQ ID NO: 29 | miR-18a-5p | UAAGGUGCAUCUA |
| | | GUGCAGAUAG |
| SEQ ID NO: 30 | miR-20a-3p | ACUGCAUUAUGAG |
| | | CACUUAAAG |
| SEQ ID NO: 31 | miR-20a-5p | UAAAGUGCUUAUA |
| | | GUGCAGGUAG |
| SEQ ID NO: 32 | miR-24-3p | UGGCUCAGUUCAG |
| | | CAGGAACAG |
| SEQ ID NO: 33 | miR-24-1-5p | UGCCUACUGAGCU |
| | | GAUAUCAGU |
| SEQ ID NO: 34 | miR-24-2-5p | UGCCUACUGAGCU |
| | | GAAACACAG |
| SEQ ID NO: 35 | miR-30e-3p | CUUUCAGUCGGAU |
| | | GUUUACAGC |
| SEQ ID NO: 36 | miR-30e-5p | UGUAAACAUCCUU |
| | | GACUGGAAG |
| SEQ ID NO: 37 | miR-93-3p | ACUGCUGAGCUAG |
| | | CACUUCCCG |
| SEQ ID NO: 38 | miR-93-5p | CAAAGUGCUGUUC |
| | | GUGCAGGUAG |
| SEQ ID NO: 39 | miR-103a-3p | AGCAGCAUUGUAC |
| | | AGGGCUAUGA |
| SEQ ID NO: 40 | miR-103a-2-5p | AGCUUCUUUACAG |
| | | UGCUGCCUUG |
| SEQ ID NO: 41 | miR-103b | UCAUAGCCCUGUA |
| | | CAAUGCUGCU |
| SEQ ID NO: 42 | miR-106a-3p | CUGCAAUGUAAGC |
| | | ACUUCUUAC |
| SEQ ID NO: 43 | miR-106a-5p | AAAAGUGCUUACA |
| | | GUGCAGGUAG |
| SEQ ID NO: 44 | miR-106b-3p | CCGCACUGUGGGU |
| | | ACUUGCUGC |
| SEQ ID NO: 45 | miR-106b-5p | UAAAGUGCUGACA |
| | | GUGCAGAU |
| SEQ ID NO: 46 | miR-107 | AGCAGCAUUGUAC |
| | | AGGGCUAUCA |
| SEQ ID NO: 47 | miR-125a-3p | ACAGGUGAGGUUC |
| | | UUGGGAGCC |
| SEQ ID NO: 48 | miR-125a-5p | UCCCUGAGACCCU |
| | | UUAACCUGUGA |
| SEQ ID NO: 49 | miR-125b-1-3p | ACGGGUUAGGCUC |
| | | UUGGGAGCU |
| SEQ ID NO: 50 | miR-125b-2-3p | UCACAAGUCAGGC |
| | | UCUUGGGAC |
| SEQ ID NO: 51 | miR-125b-5p | UCCCUGAGACCCU |
| | | AACUUGUGA |
| SEQ ID NO: 52 | miR-128-3p | UCACAGUGAACCG |
| | | GUCUCUUU |
| SEQ ID NO: 53 | miR-128-1-5p | CGGGGCCGUAGCA |
| | | CUGUCUGAGA |
| SEQ ID NO: 54 | miR-128-2-5p | GGGGGCCGAUACA |
| | | CUGUACGAGA |
| SEQ ID NO: 55 | miR-133a-3p | UUUGGUCCCCUUC |
| | | AACCAGCUG |
| SEQ ID NO: 56 | miR-133a-5p | AGCUGGUAAAAUG |
| | | GAACCAAAU |
| SEQ ID NO: 57 | miR-133b | UUUGGUCCCCUUC |
| | | AACCAGCUA |
| SEQ ID NO: 58 | miR-141-3p | UAACACUGUCUGG |
| | | UAAAGAUGG |
| SEQ ID NO: 59 | miR-141-5p | CAUCUUCCAGUAC |
| | | AGUGUUGGA |
| SEQ ID NO: 60 | miR-149-3p | AGGGAGGGACGGG |
| | | GGCUGUGC |
| SEQ ID NO: 61 | miR-149-5p | UCUGGCUCCGUGU |
| | | CUUCACUCCC |
| SEQ ID NO: 62 | miR-182-3p | UGGUUCUAGACUU |
| | | GCCAACUA |
| SEQ ID NO: 63 | miR-182-5p | UUUGGCAAUGGUA |
| | | GAACUCACACU |
| SEQ ID NO: 64 | miR-188-3p | CUCCCACAUGCAG |
| | | GGUUUGCA |
| SEQ ID NO: 65 | miR-188-5p | CAUCCCUUGCAUG |
| | | GUGGAGGG |
| SEQ ID NO: 66 | miR-198 | GGUCCAGAGGGGA |
| | | GAUAGGUUC |
| SEQ ID NO: 67 | miR-200a-3p | UAACACUGUCUGG |
| | | UAACGAUGU |
| SEQ ID NO: 68 | miR-200a-5p | CAUCUUACCGGAC |
| | | AGUGCUGGA |
| SEQ ID NO: 69 | miR-200b-3p | UAAUACUGCCUGG |
| | | UAAUGAUGA |
| SEQ ID NO: 70 | miR-200b-5p | CAUCUUACUGGGC |
| | | AGCAUUGGA |
| SEQ ID NO: 71 | miR-204-3p | GCUGGGAAGGCAA |
| | | AGGGACGU |
| SEQ ID NO: 72 | miR-204-5p | UUCCCUUUGUCAU |
| | | CCUAUGCCU |
| SEQ ID NO: 73 | miR-206 | UGGAAUGUAAGGA |
| | | AGUGUGUGG |
| SEQ ID NO: 74 | miR-221-3p | AGCUACAUUGUCU |
| | | GCUGGGUUUC |
| SEQ ID NO: 75 | miR-221-5p | ACCUGGCAUACAA |
| | | UGUAGAUUU |
| SEQ ID NO: 76 | miR-296-3p | GAGGGUUGGGUGG |
| | | AGGCUCUCC |
| SEQ ID NO: 77 | miR-296-5p | AGGGCCCCCCCUC |
| | | AAUCCUGU |
| SEQ ID NO: 78 | miR-324-5p | CGCAUCCCCUAGG |
| | | GCAUUGGUGU |
| SEQ ID NO: 79 | miR-326 | CCUCUGGGCCCUU |
| | | CCUCCAG |
| SEQ ID NO: 80 | miR-330-3p | GCAAAGCACACGG |
| | | CCUGCAGAGA |
| SEQ ID NO: 81 | miR-331-3p | GCCCCUGGGCCUA |
| | | UCCUAGAA |
| SEQ ID NO: 82 | miR-331-5p | CUAGGUAUGGUCC |
| | | CAGGGAUCC |
| SEQ ID NO: 83 | miR-340-3p | UCCGUCUCAGUUA |
| | | CUUUAUAGC |
| SEQ ID NO: 84 | miR-340-5p | UUAUAAAGCAAUG |
| | | AGACUGAUU |
| SEQ ID NO: 85 | miR-345-3p | GCCCUGAACGAGG |
| | | GGUCUGGAG |
| SEQ ID NO: 86 | miR-345-5p | GCUGACUCCUAGU |
| | | CCAGGGCUC |
| SEQ ID NO: 87 | miR-374a-3p | CUUAUCAGAUUGU |
| | | AUUGUAAUU |
| SEQ ID NO: 88 | miR-374a-5p | UUAUAAUACAACC |
| | | UGAUAAGUG |
| SEQ ID NO: 89 | miR-374b-3p | CUUAGCAGGUUGU |
| | | AUUAUCAUU |
| SEQ ID NO: 90 | miR-374b-5p | AUAUAAUACAACC |
| | | UGCUAAGUG |
| SEQ ID NO: 91 | miR-374c-3p | CACUUAGCAGGUU |
| | | GUAUUAUAU |
| SEQ ID NO: 92 | miR-374c-5p | AUAAUACAACCUG |
| | | CUAAGUGCU |
| SEQ ID NO: 93 | miR-384 | AUUCCUAGAAAUU |
| | | GUUCAUA |
| SEQ ID NO: 94 | miR-412-3p | ACUUCACCUGGUC |
| | | CACUAGCCGU |
| SEQ ID NO: 95 | miR-412-5p | UGGUCGACCAGUU |
| | | GGAAAGUAAU |
| SEQ ID NO: 96 | miR-422a | ACUGGACUUAGGG |
| | | UCAGAAGGC |
| SEQ ID NO: 97 | miR-449a | UGGCAGUGUAUUG |
| | | UUAGCUGGU |
| SEQ ID NO: 98 | miR-449b-3p | CAGCCACAACUAC |
| | | CCUGCCACU |
| SEQ ID NO: 99 | miR-449b-5p | AGGCAGUGUAUUG |
| | | UUAGCUGGC |
| SEQ ID NO: 100 | miR-449c-3p | UUGCUAGUUGCAC |
| | | UCCUCUCUGU |
| SEQ ID NO: 101 | miR-449c-5p | UAGGCAGUGUAUU |
| | | GCUAGCGGCUGU |
| SEQ ID NO: 102 | miR-485-3p | GUCAUACACGGCU |
| | | CUCCUCUCU |
| SEQ ID NO: 103 | miR-509-3p | UGAUUGGUACGUC |
| | | UGUGGGUAG |
| SEQ ID NO: 104 | miR-509-5p | UACUGCAGACAGU |
| | | GGCAAUCA |
| SEQ ID NO: 105 | miR-509-3-5p | UACUGCAGACGUG |
| | | GCAAUCAUG |
| SEQ ID NO: 106 | miR-617 | AGACUUCCCAUUU |
| | | GAAGGUGGC |
| SEQ ID NO: 107 | miR-625-3p | GACUAUAGAACUU |
| | | UCCCCCUCA |
| SEQ ID NO: 108 | miR-625-5p | AGGGGGAAAGUUC |
| | | UAUAGUCC |
| SEQ ID NO: 109 | miR-765 | UGGAGGAGAAGGA |
| | | AGGUGAUG |

The examples and embodiments described herein are for illustrative purposes only and various modifications or changes suggested to persons skilled in the art are to be included within the spirit and purview of this application and scope of the appended claims.

What is claimed is:

1. A pharmaceutical composition comprising:
a) a nucleic acid polymer that comprises at least 80% sequence identity to a nucleic acid sequence selected from SEQ ID NOs: 1-5 and 114-119, and wherein the nucleic acid polymer is at most 100 nucleotides in length; and
b) a pharmaceutically acceptable excipient and/or a delivery vehicle.

2. The pharmaceutical composition of claim 1, wherein the nucleic acid polymer comprises at least 85%, 90%, 95%, 99% or 100% sequence identity to a nucleic acid sequence selected from SEQ ID NOs: 1-5 and 114-119, and wherein the nucleic acid polymer is at most 100 nucleotides in length.

3. The pharmaceutical composition of claim 1, wherein the nucleic acid polymer hybridizes to a target sequence of the truncated TrkC or truncated TrkB mRNA.

4. The pharmaceutical composition of claim 1, wherein the nucleic acid polymer induces a decrease in a truncated TrkC or truncated TrkB expression level or a decrease in truncated TrkC or truncated TrkB activity.

5. The pharmaceutical composition of claim 4, wherein the decrease in truncated TrkC or truncated TrkB activity correlates to a decrease in TNF-α production.

6. The pharmaceutical composition of claim 1, wherein the nucleic acid polymer comprising at least 80%, 85%, 90%, 95%, or 99% sequence identity to a nucleic acid sequence selected from SEQ ID NOs: 1-5 hybridizes to a target sequence of the truncated TrkC.

7. The pharmaceutical composition of claim 1, wherein the nucleic acid polymer comprising at least 80%, 85%, 90%, 95%, or 99% sequence identity to a nucleic acid sequence selected from SEQ ID NOs: 114-119 hybridizes to a target sequence of the truncated TrkB.

8. The pharmaceutical composition of claim 1, wherein the nucleic acid polymer is further modified at the nucleoside moiety, at the phosphate moiety, or a combination thereof.

9. The pharmaceutical composition of claim 1, wherein the nucleic acid polymer further comprises one or more artificial nucleotide bases.

10. The pharmaceutical composition of claim 9, wherein the one or more artificial nucleotide bases comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, locked nucleic acid (LNA), ethylene nucleic acid (ENA), peptide nucleic acid (PNA), l′, 5′-anhydrohexitol nucleic acids (HNA), morpholino, methylphosphonate nucleotides, thiolphosphonate nucleotides, or 2′-fluoro N3-P5′-phosphoramidites.

11. The pharmaceutical composition of claim 1, wherein the nucleic acid polymer comprises a short hairpin RNA (shRNA) molecule, a microRNA (miRNA) molecule, or a siRNA molecule.

12. The pharmaceutical composition of claim 1, wherein the nucleic acid polymer further comprises a complement nucleic acid polymer to form a double stranded RNA molecule.

13. The pharmaceutical composition of claim 1, wherein the nucleic acid polymer is at most 10, 15, 20, 25, 30, 35, 40, 45, 50, 55, 60, 70, 80, 90, or 100 nucleotides in length.

14. The pharmaceutical composition of claim 1, wherein the pharmaceutical composition comprising the nucleic acid polymer is administered to a patient in need thereof as an intramuscular, intrathecal, intravitreal, intraconjunctival, intravenous or subcutaneous administration.

15. The pharmaceutical composition of claim 1, further comprising a vector that comprises the nucleic acid polymer.

16. The pharmaceutical composition of claim 15, wherein the vector is a viral vector.

17. The pharmaceutical composition of claim 3, wherein the truncated TrkC is a non-catalytic truncated TrkC.

18. The pharmaceutical composition of claim 3, wherein the truncated TrkC protein comprises at least 80%, 85%, 90%, 95%, or 99% sequence identity to the amino acid sequence of SEQ ID NO: 10 or 113.

19. The pharmaceutical composition of claim 3, wherein the truncated TrkC is TrkC.T1.

20. The pharmaceutical composition of claim 3, wherein the truncated TrkB is a non-catalytic truncated TrkB.

21. The pharmaceutical composition of claim 3, wherein the truncated TrkB protein comprises at least 80%, 85%, 90%, 95%, or 99% sequence identity to an amino acid sequence selected from SEQ ID NOs: 11-12 and 121-123.

22. The pharmaceutical composition of claim 3, wherein the truncated TrkB is TrkB.T1.

23. The pharmaceutical composition of claim 1, wherein the pharmaceutical composition is administered for the treatment of a non-otic disease or condition associated with an elevated expression level of truncated TrkC or truncated TrkB, or an elevated activity of truncated TrkC or truncated TrkB.

24. A pharmaceutical composition comprising:
a) a nucleic acid polymer that hybridizes to a target sequence comprising a binding motif selected from CCAAUC, CUCCAA, or ACUGUG, wherein the binding motif is located in a sequence encoding a truncated TrkC, and wherein the nucleic acid polymer is at most 100 nucleotides in length; and
b) a pharmaceutically acceptable excipient and/or a delivery vehicle.

25. The pharmaceutical composition of claim 24, wherein the nucleic acid polymer hybridizes to a target sequence that is located at the 3′UTR region of the truncated TrkC mRNA.

26. The pharmaceutical composition of claim 24, wherein the nucleic acid polymer comprises a short hairpin RNA (shRNA) molecule, a microRNA (miRNA) molecule, a siRNA molecule, or a double stranded RNA molecule.

27. The pharmaceutical composition of claim 24, wherein the nucleic acid polymer is a shRNA molecule.

28. The pharmaceutical composition of claim 24, wherein the truncated TrkC is a non-catalytic truncated TrkC.

29. The pharmaceutical composition of claim 24, wherein the truncated TrkC is TrkC.T1.

30. A method of treating a non-otic disease or condition associated with an elevated expression level or activity level of truncated TrkC or truncated TrkB, comprising administering to a patient having a non-otic disease or condition a therapeutic amount of a pharmaceutical composition comprising:
a nucleic acid polymer that comprises at least 80%, 85%, 90%, 95%, 99% or 100% sequence identity to a nucleic acid sequence selected from SEQ ID NOs: 1-5 and 114-119, and wherein the nucleic acid polymer is at most 100 nucleotides in length;
a small molecule antagonist of a truncated TrkC or truncated TrkB; or
a polypeptide antagonist of a truncated TrkC or truncated TrkB; and
a pharmaceutically acceptable excipient and/or a delivery vehicle.

31. The method of claim 30, wherein the small molecule is a peptidomimetic.

32. The method of claim 30, wherein the small molecule is a small molecule as illustrated in FIG. 2.

33. The method of claim 30, wherein the polypeptide antagonist is an antibody or binding fragment thereof.

34. The method of claim 30, wherein the non-otic disease or condition comprises a neurodegenerative disease or a symptomatic or pre-symptomatic condition with alterations to synapses.

35. The method of claim 34, wherein the neurodegenerative disease comprises polyglutamine expansion disorder, fragile X syndrome, fragile XE mental retardation, Friedreich's ataxia, myotonic dystrophy, spinocerebellar ataxia type 8, and spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, glaucoma, ischemia stroke, Krabbe disease, Lewy body dementia, multiple sclerosis, multiple system atrophy, Parkinson's disease, Pelizaeus-Merzbacher disease, Pick's disease, primary lateral sclerosis, progressive muscular atrophy, progressive bulbar palsy, pseudobulbar palsy, retinitis pigmentosa, Refsum's disease, Sandhoff disease, Schilder's disease, spinal cord injury (SCI), spinal muscular atrophy (SMA), Steele-Richardson-Olszewski disease, and Tabes dorsalis.

36. The method of claim 35, wherein the polyglutamine repeat disease is Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17).

37. The method of claim 34, wherein the non-otic disease or condition comprises amyotrophic lateral sclerosis (ALS), spinal muscular atrophy (SMA), glaucoma, spinal cord injury (SCI), or retinitis pigmentosa.

38. The method of claim 30, wherein the non-otic disease or condition comprises a psychiatric disorder.

39. The method of claim 30, wherein the pharmaceutical composition is administered for intramuscular, intrathecal, intravitreal, intraconjunctival, intravenous or subcutaneous administration.

40. The method of claim 30, wherein the pharmaceutical composition modulates the splicing enhancer elements.

41. The method of claim 40, wherein the modulation comprises alternative splicing of the truncated TrkC or truncated TrkB gene.

42. The method of claim 40, wherein the modulation comprises exon skipping or introduction of mutations within an exon.
